SEQUENCE LISTING (30 sequences)

SEQ ID NO: 1 - DNA, 16996 bp, synthetic construct
  source 1..16996: mol_type = other DNA; note = Expression vector with full ORFeus and no amiR
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggagatcggtacttcgcgaatgcgtcgagatttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatgaattcccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgtcaggctggtgaaagggcaggcagcactttcccagcacactggccaaagccaacacggtagccgtccccctggcagtgacctgttatgatgacttcatcgccgtccagcaggaaggtcctggtctgcccctgctccacatcgatggcctttgttcccttccaggacagttccagcatggagccaaagctttcagggtctgatccactgatggttccagaagccaagaggtccccaggtctcaggttgcatccattaacagagtggtgtgtgagttgctgcagcatggtccagtacatgtgcttaaagttagacctgcagatggtagccgcctggctcattccttctcctttcaaagagacagacaggttgatatcaaatgtgtagggctggctgtggcagagatatggcaagggcttggggtcctgctttgggtttggcaccacaaagggcatgagggcatccataggcaccacccacggggagattgtggttccaaagcttttccccaggaatggcccaagtgggacgtactcccattgctggatgtctcgtgcgctccagtcgttcatgaggaccatcccgaaaatgtgttcatgggctttggaaatggggattggctctccgaatctgttcccagggcctacgaagaaagccatttccaactccatgtctaagagtctgcaggcaccatacacaggaggctttgagttatcaggtctcatctgccccatgggtcttcgaatcggggttccagataccacaatggaggaagctcggccatggtatcccacaggtaagtggagccaatttggcaacagcgcattctccttgcctctgaacataatgccaacattggtggcatgctgccgagaagagtagaagtccgtgtagtctcctatggtagcaggaaggtgcattgtcgcagaagcctgggaggtgaatgcacgctgccgaagctccttgtcatctctgagccgggcttggctggcagacagtaagttctgtaaggatgctcttgcctccttccatgcagcttgacccagacccatgaagttattgagagttgtctcatcgaagacatgttgatgtttggaaagggcaggtccggtaaagaggtgtttaatgacactcaggtccaggatctggtcaccgatggctacaccaatccgtggctttgggttgctttgagtggagaaaacaccatagggcaggttttggatgggaaagtcggagtcctcggccactggaataaaggacattgggccaggattctcctccacgtcaccgcatgttagaagacttcctctgccctctcctccggacttgtacagctcgtccatgccgccggtggagtggcggccctcggcgcgctcgtactgttccacgatggtgtagtcctcgttgtgggaggtgatgtccagcttgatgttgacgttgtaggcgccgggcagctgcacgggcttcttggccttgtaggtggtcttgacctcggcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcacctgaaatggaagaaaaaaacttcgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgtgaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagcaccctgcagcagggccgggaagcggccatctttccgctcacgcaactggtgccgaccgggccagccttgccgcccagggcggggcgatacacggcggcgcgaggccaggcaccagagcaggccatccgcggccccaaaaaccgaaaagaccttggcccgcatttacaagactatcttgccagaaaaaaagcgtcgcagcaggtcatcgtcgactggctagagagctcacttatcgaaagcagcgagacaggcgcgaaggtgcctctagaccagattcaccggtgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccatcgatacacggcggaccgtgaccacttaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccatggtcttgagctgaagaggacagcagtggagagcgcctctaacgggtgcggccgagcggaggacttttctcccgtagcctggcagccaggcgcctggagcactgcctgactaaaacccagatgccgagtggccgccggccccttagccctgcccctcgctcctggctaacatggccgaaaggtcgtattctccgggggaggacgggggcccggagaggagggggtggagtgcctggttttccagtcaggcggccggagggcagccctcaagaacggccctgaccgcccgcggggtgaggggccctttctgggcaggacccgccccttggtcccgcagagccttggtacttggacctgaaccttgctccgagagggagtcctcgcggacgtcagcgctgcggccgcgtatacgtttaaacggaacttaggaaattagtctgaacaggtgagagggtgcgccagagaacctgacagcttctggaacaggcggaagcacagaggcgctgaggcatcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctttgtgggccggggacagccagccaccatccggaccggaggacaggtgcccgcccggctggggaggcggcctaagccacagcagcagcggtcgccatcttggtccgagacccgccgaacttaggaaattagtctgaacaggtgagagggtgcgccagagaacctgacagcttctagaacaggccgaagcacagaggcgctgaggcagcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcgacctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctgtgtgggccggggacagccagccaccttcctgaccggaggacaggtgcccacccggcaggggaggcggcctaagccacagcagcagcggtcgccatcttggtcccgggactccaaggaacttaggaatttagtctgcttaggtgagagtctgtaccacctgggaactgccaaagcaacacagtgtctgagaaaggtcctgttttgggccttcttcttcgtccaggaggaggtccaaatacaagatatctgcgcaccttccctgtaagagagcttgccagcagagagtgctctgagcactgaaactcagaggagagaatctgtctcccaggtctgctgatagacggtaacagaatcaccagaagaacaatctctaaacagagtcaactataactactaactccagagattaccagtttaaacgtatacggccggccggcgcgccgccaccatggccaagggcaagcgccgcaacctgaccaaccgcaaccaggaccacagccccagccccgagcccagcacccccaccagccccagccccggcaaccccaacacccccgagaacctggacctggacctgaaggcctacctgatgatgatggtggaggacatcaagaaggacttcaacaagagcctgaaggagatccaggagaacaccgccaaggagctgcaggtgctgaaggagaagcaggagaacaccatcaagcaggtggaggtgctgaccgagaaggaggagaagacctacaagcaggtgatggagatgaacaagaccatcctggacctgaagcgcgaggtcgacaccatcaagaagacccagagcgaggccaccctggagatcgagaccctgggcaagaagagcggcaccatcgacctgagcatcagcaaccgcatccaggagatggaggagcgcatcagcggcgccgaggacagcatcgagaacatcggcaccaccatcaaggagaacggcaagtgcaagaagatcctgacccagaacatccaggagatccaggacaccatccgccgccccaacgtgcgcatcatcggcgtggacgagaacgaggacttccagctgaagggccccgccaacatcttcaacaagatcatcgaggagaacttccccaacctgaagaacgagatgcacatgaacatccaggaggcctaccgcacccccaaccgcctggaccagaagcgcaacagctctagacacatcatcatccgcaccagcaacgccctgaacaaggaccgcatcctgaaggccgtgcgcgagaagggccaggtgacctacaagggcaagcccatccgcatcacccccgacttcagccccgagaccatgaaggcccgccgcgcctggaccgacgtgatccagaccctgcgcgagcacaagctgcagccccgcctgctgtaccccgccaagctgagcatcatcatcgagggcgagaccaaggtgttccacgacaagaccaagttcacccactacctgagcaccaaccccgccctgcagcgcatcatcaccgagaagaaccagtacaagaacggcaacaacgccctggagaagacccgccgctaatccctcaacaaaccttaattaagacagccacaagaacagaatgccccccctgaccaccaagatcaccggcagcaacaactacttcagcctgatcagcctgaacatcaacggcctgaacagccccatcaagcggcaccgcctgaccaactggctgcacaagcaggaccccaccttctgttgcctccaggagacccacctgcgcgagaaggaccggcactacctgcggatgaagggctggaagaccatctttcaggccaacggcatgaagaagcaggctggcgtggccatcctgatcagcgacaagatcgacttccagcccaaggtgatcaagaaggacaaggagggccacttcatcctgatcaagggcaagatcctgcaggaggagctgagcattctgaacatctacgcccccaacacccgcgccgccaccttcaccaaggagaccctcgtgaagctgaaggcccacatcgctccccacaccatcatcgtcggcgacttcaacacccccctgagccccatggacagatcttggaagcagaagctgaaccgcgacaccctgaagctgaccgaggtgatgaagcagatggacctgaccgacatctaccgcaccttctaccccaagaccaagggctacaccttcttcagcgctccccacggcaccttcagcaagatcgaccacatcatcggccacaagagcgggctgaaccggctgaagaacatcgagatcgtgccctgcatcctgagcgaccaccatgccctgcgcctgatcttcaacaacaagattaacaaccgcaagcccaccttcacctggaagctgaacaacaccctgctgaacgacaccctggtgaaggaaggcatcaagaaggagatcaaggacttcctggagttcaacgagaacgaggccaccacctaccctaacctgtgggacaccatgaaggccttcctgcggggcaagctgatcgccatgagcgccttcaagaagaagcgggagcgcgcccacactagtagcctgaccacccacctgaaggccctggagaagaaggaggctaactcccccaagcgctcccggcggcaggagatcatcaagctgcgcggcgagatcaaccaggtggagacccggcgcaccatccaacggatcaaccagacccggtcttggttcttcgagaagatcaacaagatcgacaagcccctggctcgcctgaccaagggccaccgcgacaagatcctgatcaacaagatccgcaacgagaagggcgacatcaccaccgaccccgaggagatccagaacaccatccgcagcttctacaagcgcctgtacagcaccaagctggagaacctggacgagatggacaagttcctggaccgctatcaggtgcccaagctgaaccaggaccaggtggacctgctgaacagccccatctcccccaaggaaatcgaggccgtgatcaacagcctgcccgccaagaagagccccggccccgacggcttcagcgctgagttctaccagaccttcaaggaggacctgacccccgtgctgcacaagcttttccacaagatcgaggtcgagggcatcctccccaacagcttctacgaggccaccatcaccctgatccccaagccccagaaggaccccaccaagatcgagaacttccgccccatcagcctgatgaacatcgacgccaagatcctgaacaagatcctcgccaaccgcatccaggagcacatcaaggccatcatccaccctgaccaggtcggcttcatccccggcatgcagggctggttcaacatccgcaagagcatcaacgtgatccactacatcaacaagctgaaggacaagaaccacatgatcatcagcctggacgccgagaaggccttcgacaagatccagcaccccttcatgatcaaggtcctggagcgcagcggcattcagggccagtacctcaacatgatcaaggccatctacagcaagcccgtcgccaacatcaaggtgaacggggagaagctggaggccatccctctgaagagcggcacgcgtcagggctgtcctctgtccccctacctgttcaacatcgtgctggaggtgctggcccgcgctatccggcagcagaaggagatcaagggcatccagatcggcaaggaggaagtgaagatcagcctgttcgccgacgacatgatcgtgtacatcagcgaccccaagaacagcaaccgcgagctcctgaacctgatcaacagcttcggcgaggtggctggctacaagatcaacagcaacaagagcatggccttcctgtacaccaagaacaagcaggccgagaaggagatccgcgagaccacccccttcagcatcgccaccaacaacatcaagtacctgggcgtgaccctgaccaaggaggtgaaggacctgtacgacaagaacttcaagagcctgaagaaggagatcaaggaggacctgcgccgctggaaggacctgccctgcagctggatcggccgcaccaacatcgtgaagatggccatcctgcccaaggctatctaccgcttcaacgccatccccatcaagatccccacccagttcttcaacgagctcgagggcgccatctgcaagttcatctggaacaacaagaagccccgcatcgccaagaccctgctgaaggacaagcgcaccagtggggggatcaccatgcccgacctgaagctgtactaccgcgccatcgtgatcaagaccgcctggtactggtaccgcgaccgccaggtggaccagtggaaccgcatcgaggaccccgagatgaacccccacacctacggccacctgatcttcgacaagggcgccaagaccatccagtggaagaaggacagcatcttcaacaactggtgctggcacaactggctgctgagctgccgccgcatgcgcatcgacccctacctaagcccctgcaccaaggtgaagagcaagtggatcaaggagctgcacatcaagcccgagactctgaagctgatcgaggagaaggtgggcaagagcctggaggacatgggcaccggcgaaaagttcctgaaccgcaccgccatggcctgtgccgtgcgcagccggatcgataagtgggacctgatgaagctgcagtccttctgtaaggccaaggataccgtgtacaagaccaagcgccccccgaccgactgggagcgcatcttcacctaccccaagagcgaccgcggcctgatcagcaacatctacaaggagctgaagaaggtggacctgcgcaagagcaacaacccgctgaagaagtggggctccgagctgaacaaggagttctcccccgaggagtaccgcatggccgagaagcacctgaagaagtgcagcaccagcctgatcatccgcgagatgcagatcaagaccaccctgcgcttccacctgacccccgtgcggatggccaagatcaagaacagcggcgactcgcgatgctggcggggctgcggcgagcgcggcaccctgctgcactgctggtgggactgtcgcctggtccagcccctgtggaagagcgtgtggcggttcctgcggaagctggacatcgtgctgcccgaggaccccgctatccccctgctgggcatctaccctgaggaggcccccaccggcaagaaggacacctgcagtaccatgttcatcgccgctctgttcatcatcgcccgcaactggaaggagccccgctgccccagcaccgaggagtggattcagaagatgtggtacatctacaccatggagtactacagcgccatcaagaagaacgagttcatgaagttcctggccaagtggatggacctggagagcatcatcctgagcgaggtgacccagagccagcgcaacagccacaacatgtacagcctgatcagcggctactagcctaaaaccggtgatacccacgatataagatacaattgcctaaacacatgaaactcaagaaaaatgaagactgaagtgtggacactatgcccctccttagaagtggggacaaaacagccatggaaggagttacagaaataaagtttggagctgagatgaaaggagggaccatgtagagactgccatatccagggttccaccccataatcagcatccaagctctgacaccattgcatatactaggaagattttatcgaaaggacccagatgtagctgtctcttgtgagactatgcggggcctagcaacacagaagtggatgctcacagtcagctaatggatggatcacagggctttcaatggaggagctagagaaagtacccaaggagctaaagggatctgcaactctataggtggaacaacattatgagtttacggatccgaattctcgacggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgtgtgtgtgcgcgcacgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgaagcttgagctcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaaattcctgcagcccgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgatcgtatcgcgatcgctatcagatctataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaatcggatgccgggaccgacgagtgcagaggcgtgcaagcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 13120 bp, synthetic construct
  source 1..13120: mol_type = other DNA; note = Expression vector with ORF1, TF monomer, and without amiR
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggagatcggtacttcgcgaatgcgtcgagatttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatgaattcccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgtcaggctggtgaaagggcaggcagcactttcccagcacactggccaaagccaacacggtagccgtccccctggcagtgacctgttatgatgacttcatcgccgtccagcaggaaggtcctggtctgcccctgctccacatcgatggcctttgttcccttccaggacagttccagcatggagccaaagctttcagggtctgatccactgatggttccagaagccaagaggtccccaggtctcaggttgcatccattaacagagtggtgtgtgagttgctgcagcatggtccagtacatgtgcttaaagttagacctgcagatggtagccgcctggctcattccttctcctttcaaagagacagacaggttgatatcaaatgtgtagggctggctgtggcagagatatggcaagggcttggggtcctgctttgggtttggcaccacaaagggcatgagggcatccataggcaccacccacggggagattgtggttccaaagcttttccccaggaatggcccaagtgggacgtactcccattgctggatgtctcgtgcgctccagtcgttcatgaggaccatcccgaaaatgtgttcatgggctttggaaatggggattggctctccgaatctgttcccagggcctacgaagaaagccatttccaactccatgtctaagagtctgcaggcaccatacacaggaggctttgagttatcaggtctcatctgccccatgggtcttcgaatcggggttccagataccacaatggaggaagctcggccatggtatcccacaggtaagtggagccaatttggcaacagcgcattctccttgcctctgaacataatgccaacattggtggcatgctgccgagaagagtagaagtccgtgtagtctcctatggtagcaggaaggtgcattgtcgcagaagcctgggaggtgaatgcacgctgccgaagctccttgtcatctctgagccgggcttggctggcagacagtaagttctgtaaggatgctcttgcctccttccatgcagcttgacccagacccatgaagttattgagagttgtctcatcgaagacatgttgatgtttggaaagggcaggtccggtaaagaggtgtttaatgacactcaggtccaggatctggtcaccgatggctacaccaatccgtggctttgggttgctttgagtggagaaaacaccatagggcaggttttggatgggaaagtcggagtcctcggccactggaataaaggacattgggccaggattctcctccacgtcaccgcatgttagaagacttcctctgccctctcctccggacttgtacagctcgtccatgccgccggtggagtggcggccctcggcgcgctcgtactgttccacgatggtgtagtcctcgttgtgggaggtgatgtccagcttgatgttgacgttgtaggcgccgggcagctgcacgggcttcttggccttgtaggtggtcttgacctcggcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcacctgaaatggaagaaaaaaacttcgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgtgaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagcaccctgcagcagggccgggaagcggccatctttccgctcacgcaactggtgccgaccgggccagccttgccgcccagggcggggcgatacacggcggcgcgaggccaggcaccagagcaggccatccgcggccccaaaaaccgaaaagaccttggcccgcatttacaagactatcttgccagaaaaaaagcgtcgcagcaggtcatcgtcgactggctagagagctcacttatcgaaagcagcgagacaggcgcgaaggtgcctctagaccagattcaccggtgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccatcgatacacggcggaccgtgaccacttaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccatggtcttgagctgaagaggacagcagtggagagcgcctctaacgggtgcggccgagcggaggacttttctcccgtagcctggcagccaggcgcctggagcactgcctgactaaaacccagatgccgagtggccgccggccccttagccctgcccctcgctcctggctaacatggccgaaaggtcgtattctccgggggaggacgggggcccggagaggagggggtggagtgcctggttttccagtcaggcggccggagggcagccctcaagaacggccctgaccgcccgcggggtgaggggccctttctgggcaggacccgccccttggtcccgcagagccttggtacttggacctgaaccttgctccgagagggagtcctcgcggacgtcagcgctgcggccgcgtatacgtttaaacggaacttaggaaattagtctgaacaggtgagagggtgcgccagagaacctgacagcttctggaacaggcggaagcacagaggcgctgaggcatcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctttgtgggccggggacagccagccaccatccggaccggaggacaggtgcccgcccggctggggaggcggcctaagccacagcagcagcggtcgccatcttggtccgagacccgccgaacttaggaaattagtctgaacaggtgagagggtgcgccagagaacctgacagcttctagaacaggccgaagcacagaggcgctgaggcagcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcgacctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctgtgtgggccggggacagccagccaccttcctgaccggaggacaggtgcccacccggcaggggaggcggcctaagccacagcagcagcggtcgccatcttggtcccgggactccaaggaacttaggaatttagtctgcttaggtgagagtctgtaccacctgggaactgccaaagcaacacagtgtctgagaaaggtcctgttttgggccttcttcttcgtccaggaggaggtccaaatacaagatatctgcgcaccttccctgtaagagagcttgccagcagagagtgctctgagcactgaaactcagaggagagaatctgtctcccaggtctgctgatagacggtaacagaatcaccagaagaacaatctctaaacagagtcaactataactactaactccagagattaccagtttaaacgtatacggccggccggcgcgccgccaccatggccaagggcaagcgccgcaacctgaccaaccgcaaccaggaccacagccccagccccgagcccagcacccccaccagccccagccccggcaaccccaacacccccgagaacctggacctggacctgaaggcctacctgatgatgatggtggaggacatcaagaaggacttcaacaagagcctgaaggagatccaggagaacaccgccaaggagctgcaggtgctgaaggagaagcaggagaacaccatcaagcaggtggaggtgctgaccgagaaggaggagaagacctacaagcaggtgatggagatgaacaagaccatcctggacctgaagcgcgaggtcgacaccatcaagaagacccagagcgaggccaccctggagatcgagaccctgggcaagaagagcggcaccatcgacctgagcatcagcaaccgcatccaggagatggaggagcgcatcagcggcgccgaggacagcatcgagaacatcggcaccaccatcaaggagaacggcaagtgcaagaagatcctgacccagaacatccaggagatccaggacaccatccgccgccccaacgtgcgcatcatcggcgtggacgagaacgaggacttccagctgaagggccccgccaacatcttcaacaagatcatcgaggagaacttccccaacctgaagaacgagatgcacatgaacatccaggaggcctaccgcacccccaaccgcctggaccagaagcgcaacagctctagacacatcatcatccgcaccagcaacgccctgaacaaggaccgcatcctgaaggccgtgcgcgagaagggccaggtgacctacaagggcaagcccatccgcatcacccccgacttcagccccgagaccatgaaggcccgccgcgcctggaccgacgtgatccagaccctgcgcgagcacaagctgcagccccgcctgctgtaccccgccaagctgagcatcatcatcgagggcgagaccaaggtgttccacgacaagaccaagttcacccactacctgagcaccaaccccgccctgcagcgcatcatcaccgagaagaaccagtacaagaacggcaacaacgccctggagaagacccgccgctaatccctcaacaaaccttaccggtgatacccacgatataagatacaattgcctaaacacatgaaactcaagaaaaatgaagactgaagtgtggacactatgcccctccttagaagtggggacaaaacagccatggaaggagttacagaaataaagtttggagctgagatgaaaggagggaccatgtagagactgccatatccagggttccaccccataatcagcatccaagctctgacaccattgcatatactaggaagattttatcgaaaggacccagatgtagctgtctcttgtgagactatgcggggcctagcaacacagaagtggatgctcacagtcagctaatggatggatcacagggctttcaatggaggagctagagaaagtacccaaggagctaaagggatctgcaactctataggtggaacaacattatgagtttacggatccgaattctcgacggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgtgtgtgtgcgcgcacgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgaagcttgagctcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaaattcctgcagcccgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgatcgtatcgcgatcgctatcagatctataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaatcggatgccgggaccgacgagtgcagaggcgtgcaagcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 3 - DNA, 11344 bp, synthetic construct
  source 1..11344: mol_type = other DNA; note = Expression vector with ORF1, with no amiR, and without TF monomer
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggagatcggtacttcgcgaatgcgtcgagatttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatgaattcccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgtcaggctggtgaaagggcaggcagcactttcccagcacactggccaaagccaacacggtagccgtccccctggcagtgacctgttatgatgacttcatcgccgtccagcaggaaggtcctggtctgcccctgctccacatcgatggcctttgttcccttccaggacagttccagcatggagccaaagctttcagggtctgatccactgatggttccagaagccaagaggtccccaggtctcaggttgcatccattaacagagtggtgtgtgagttgctgcagcatggtccagtacatgtgcttaaagttagacctgcagatggtagccgcctggctcattccttctcctttcaaagagacagacaggttgatatcaaatgtgtagggctggctgtggcagagatatggcaagggcttggggtcctgctttgggtttggcaccacaaagggcatgagggcatccataggcaccacccacggggagattgtggttccaaagcttttccccaggaatggcccaagtgggacgtactcccattgctggatgtctcgtgcgctccagtcgttcatgaggaccatcccgaaaatgtgttcatgggctttggaaatggggattggctctccgaatctgttcccagggcctacgaagaaagccatttccaactccatgtctaagagtctgcaggcaccatacacaggaggctttgagttatcaggtctcatctgccccatgggtcttcgaatcggggttccagataccacaatggaggaagctcggccatggtatcccacaggtaagtggagccaatttggcaacagcgcattctccttgcctctgaacataatgccaacattggtggcatgctgccgagaagagtagaagtccgtgtagtctcctatggtagcaggaaggtgcattgtcgcagaagcctgggaggtgaatgcacgctgccgaagctccttgtcatctctgagccgggcttggctggcagacagtaagttctgtaaggatgctcttgcctccttccatgcagcttgacccagacccatgaagttattgagagttgtctcatcgaagacatgttgatgtttggaaagggcaggtccggtaaagaggtgtttaatgacactcaggtccaggatctggtcaccgatggctacaccaatccgtggctttgggttgctttgagtggagaaaacaccatagggcaggttttggatgggaaagtcggagtcctcggccactggaataaaggacattgggccaggattctcctccacgtcaccgcatgttagaagacttcctctgccctctcctccggacttgtacagctcgtccatgccgccggtggagtggcggccctcggcgcgctcgtactgttccacgatggtgtagtcctcgttgtgggaggtgatgtccagcttgatgttgacgttgtaggcgccgggcagctgcacgggcttcttggccttgtaggtggtcttgacctcggcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcacctgaaatggaagaaaaaaacttcgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgtgaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagcaccctgcagcagggccgggaagcggccatctttccgctcacgcaactggtgccgaccgggccagccttgccgcccagggcggggcgatacacggcggcgcgaggccaggcaccagagcaggccatccgcggccccaaaaaccgaaaagaccttggcccgcatttacaagactatcttgccagaaaaaaagcgtcgcagcaggtcatcgtcgactggctagagagctcacttatcgaaagcagcgagacaggcgcgaaggtgcctctagaccagattcaccggtgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccatcgatacacggcggaccgtgaccacttaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccatggtcttgagctgaagaggacagcagtggagagcgcctctaacgggtgcggccgagcggaggacttttctcccgtagcctggcagccaggcgcctggagcactgcctgactaaaacccagatgccgagtggccgccggccccttagccctgcccctcgctcctggctaacatggccgaaaggtcgtattctccgggggaggacgggggcccggagaggagggggtggagtgcctggttttccagtcaggcggccggagggcagccctcaagaacggccctgaccgcccgcggggtgaggggccctttctgggcaggacccgccccttggtcccgcagagccttggtacttggacctgaaccttgctccgagagggagtcctcgcggacgtcagcgctgcggccgcgtgtggatccgtgtactagttatcgcggccgctatcggccggccggcgcgccgccaccatggccaagggcaagcgccgcaacctgaccaaccgcaaccaggaccacagccccagccccgagcccagcacccccaccagccccagccccggcaaccccaacacccccgagaacctggacctggacctgaaggcctacctgatgatgatggtggaggacatcaagaaggacttcaacaagagcctgaaggagatccaggagaacaccgccaaggagctgcaggtgctgaaggagaagcaggagaacaccatcaagcaggtggaggtgctgaccgagaaggaggagaagacctacaagcaggtgatggagatgaacaagaccatcctggacctgaagcgcgaggtcgacaccatcaagaagacccagagcgaggccaccctggagatcgagaccctgggcaagaagagcggcaccatcgacctgagcatcagcaaccgcatccaggagatggaggagcgcatcagcggcgccgaggacagcatcgagaacatcggcaccaccatcaaggagaacggcaagtgcaagaagatcctgacccagaacatccaggagatccaggacaccatccgccgccccaacgtgcgcatcatcggcgtggacgagaacgaggacttccagctgaagggccccgccaacatcttcaacaagatcatcgaggagaacttccccaacctgaagaacgagatgcacatgaacatccaggaggcctaccgcacccccaaccgcctggaccagaagcgcaacagctctagacacatcatcatccgcaccagcaacgccctgaacaaggaccgcatcctgaaggccgtgcgcgagaagggccaggtgacctacaagggcaagcccatccgcatcacccccgacttcagccccgagaccatgaaggcccgccgcgcctggaccgacgtgatccagaccctgcgcgagcacaagctgcagccccgcctgctgtaccccgccaagctgagcatcatcatcgagggcgagaccaaggtgttccacgacaagaccaagttcacccactacctgagcaccaaccccgccctgcagcgcatcatcaccgagaagaaccagtacaagaacggcaacaacgccctggagaagacccgccgctaatccctcaacaaaccttaccggtgatacccacgatataagatacaattgcctaaacacatgaaactcaagaaaaatgaagactgaagtgtggacactatgcccctccttagaagtggggacaaaacagccatggaaggagttacagaaataaagtttggagctgagatgaaaggagggaccatgtagagactgccatatccagggttccaccccataatcagcatccaagctctgacaccattgcatatactaggaagattttatcgaaaggacccagatgtagctgtctcttgtgagactatgcggggcctagcaacacagaagtggatgctcacagtcagctaatggatggatcacagggctttcaatggaggagctagagaaagtacccaaggagctaaagggatctgcaactctataggtggaacaacattatgagtttacggatccgaattctcgacggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgtgtgtgtgcgcgcacgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgaagcttgagctcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaaattcctgcagcccgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgatcgtatcgcgatcgctatcagatctataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaatcggatgccgggaccgacgagtgcagaggcgtgcaagcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - DNA, 13453 bp, synthetic construct
  source 1..13453: mol_type = other DNA; note = Expression vector with amiR-mP53/1 element
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggagatcggtacttcgcgaatgcgtcgagatttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatgaattcccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgtcaggctggtgaaagggcaggcagcactttcccagcacactggccaaagccaacacggtagccgtccccctggcagtgacctgttatgatgacttcatcgccgtccagcaggaaggtcctggtctgcccctgctccacatcgatggcctttgttcccttccaggacagttccagcatggagccaaagctttcagggtctgatccactgatggttccagaagccaagaggtccccaggtctcaggttgcatccattaacagagtggtgtgtgagttgctgcagcatggtccagtacatgtgcttaaagttagacctgcagatggtagccgcctggctcattccttctcctttcaaagagacagacaggttgatatcaaatgtgtagggctggctgtggcagagatatggcaagggcttggggtcctgctttgggtttggcaccacaaagggcatgagggcatccataggcaccacccacggggagattgtggttccaaagcttttccccaggaatggcccaagtgggacgtactcccattgctggatgtctcgtgcgctccagtcgttcatgaggaccatcccgaaaatgtgttcatgggctttggaaatggggattggctctccgaatctgttcccagggcctacgaagaaagccatttccaactccatgtctaagagtctgcaggcaccatacacaggaggctttgagttatcaggtctcatctgccccatgggtcttcgaatcggggttccagataccacaatggaggaagctcggccatggtatcccacaggtaagtggagccaatttggcaacagcgcattctccttgcctctgaacataatgccaacattggtggcatgctgccgagaagagtagaagtccgtgtagtctcctatggtagcaggaaggtgcattgtcgcagaagcctgggaggtgaatgcacgctgccgaagctccttgtcatctctgagccgggcttggctggcagacagtaagttctgtaaggatgctcttgcctccttccatgcagcttgacccagacccatgaagttattgagagttgtctcatcgaagacatgttgatgtttggaaagggcaggtccggtaaagaggtgtttaatgacactcaggtccaggatctggtcaccgatggctacaccaatccgtggctttgggttgctttgagtggagaaaacaccatagggcaggttttggatgggaaagtcggagtcctcggccactggaataaaggacattgggccaggattctcctccacgtcaccgcatgttagaagacttcctctgccctctcctccggacttgtacagctcgtccatgccgccggtggagtggcggccctcggcgcgctcgtactgttccacgatggtgtagtcctcgttgtgggaggtgatgtccagcttgatgttgacgttgtaggcgccgggcagctgcacgggcttcttggccttgtaggtggtcttgacctcggcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcacctgaaatggaagaaaaaaacttcgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgtgaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagcaccctgcagcagggccgggaagcggccatctttccgctcacgcaactggtgccgaccgggccagccttgccgcccagggcggggcgatacacggcggcgcgaggccaggcaccagagcaggccatccgcggccccaaaaaccgaaaagaccttggcccgcatttacaagactatcttgccagaaaaaaagcgtcgcagcaggtcatcgtcgactggctagagagctcacttatcgaaagcagcgagacaggcgcgaaggtgcctctagaacacgtcgacaaagtgatttaatttataccattttaattcagctttgtaaaaatgtatcaaagagatagcaaggtattcagttttagtaaacaagataattgctcctaaagtagccccttgaagtccgaggcagtaggcactcactgcatggacgatctgtttacatctgtggcttcactaaacagatcgtccatgcagtgatcgctcactgtcaacagcaatataccttctcgagccttctgttgggttaacctgaagaagtaatcccagcaagtgtttccaagatgtgcaggcaacgattctgtaaagtactgaagcctcattcaaacagagctcacacaccggtgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccatcgatacacggcggaccgtgaccacttaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccatggtcttgagctgaagaggacagcagtggagagcgcctctaacgggtgcggccgagcggaggacttttctcccgtagcctggcagccaggcgcctggagcactgcctgactaaaacccagatgccgagtggccgccggccccttagccctgcccctcgctcctggctaacatggccgaaaggtcgtattctccgggggaggacgggggcccggagaggagggggtggagtgcctggttttccagtcaggcggccggagggcagccctcaagaacggccctgaccgcccgcggggtgaggggccctttctgggcaggacccgccccttggtcccgcagagccttggtacttggacctgaaccttgctccgagagggagtcctcgcggacgtcagcgctgcggccgcgtatacgtttaaacggaacttaggaaattagtctgaacaggtgagagggtgcgccagagaacctgacagcttctggaacaggcggaagcacagaggcgctgaggcatcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctttgtgggccggggacagccagccaccatccggaccggaggacaggtgcccgcccggctggggaggcggcctaagccacagcagcagcggtcgccatcttggtccgagacccgccgaacttaggaaattagtctgaacaggtgagagggtgcgccagagaacctgacagcttctagaacaggccgaagcacagaggcgctgaggcagcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcgacctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctgtgtgggccggggacagccagccaccttcctgaccggaggacaggtgcccacccggcaggggaggcggcctaagccacagcagcagcggtcgccatcttggtcccgggactccaaggaacttaggaatttagtctgcttaggtgagagtctgtaccacctgggaactgccaaagcaacacagtgtctgagaaaggtcctgttttgggccttcttcttcgtccaggaggaggtccaaatacaagatatctgcgcaccttccctgtaagagagcttgccagcagagagtgctctgagcactgaaactcagaggagagaatctgtctcccaggtctgctgatagacggtaacagaatcaccagaagaacaatctctaaacagagtcaactataactactaactccagagattaccagtttaaacgtatacggccggccggcgcgccgccaccatggccaagggcaagcgccgcaacctgaccaaccgcaaccaggaccacagccccagccccgagcccagcacccccaccagccccagccccggcaaccccaacacccccgagaacctggacctggacctgaaggcctacctgatgatgatggtggaggacatcaagaaggacttcaacaagagcctgaaggagatccaggagaacaccgccaaggagctgcaggtgctgaaggagaagcaggagaacaccatcaagcaggtggaggtgctgaccgagaaggaggagaagacctacaagcaggtgatggagatgaacaagaccatcctggacctgaagcgcgaggtcgacaccatcaagaagacccagagcgaggccaccctggagatcgagaccctgggcaagaagagcggcaccatcgacctgagcatcagcaaccgcatccaggagatggaggagcgcatcagcggcgccgaggacagcatcgagaacatcggcaccaccatcaaggagaacggcaagtgcaagaagatcctgacccagaacatccaggagatccaggacaccatccgccgccccaacgtgcgcatcatcggcgtggacgagaacgaggacttccagctgaagggccccgccaacatcttcaacaagatcatcgaggagaacttccccaacctgaagaacgagatgcacatgaacatccaggaggcctaccgcacccccaaccgcctggaccagaagcgcaacagctctagacacatcatcatccgcaccagcaacgccctgaacaaggaccgcatcctgaaggccgtgcgcgagaagggccaggtgacctacaagggcaagcccatccgcatcacccccgacttcagccccgagaccatgaaggcccgccgcgcctggaccgacgtgatccagaccctgcgcgagcacaagctgcagccccgcctgctgtaccccgccaagctgagcatcatcatcgagggcgagaccaaggtgttccacgacaagaccaagttcacccactacctgagcaccaaccccgccctgcagcgcatcatcaccgagaagaaccagtacaagaacggcaacaacgccctggagaagacccgccgctaatccctcaacaaaccttaccggtgatacccacgatataagatacaattgcctaaacacatgaaactcaagaaaaatgaagactgaagtgtggacactatgcccctccttagaagtggggacaaaacagccatggaaggagttacagaaataaagtttggagctgagatgaaaggagggaccatgtagagactgccatatccagggttccaccccataatcagcatccaagctctgacaccattgcatatactaggaagattttatcgaaaggacccagatgtagctgtctcttgtgagactatgcggggcctagcaacacagaagtggatgctcacagtcagctaatggatggatcacagggctttcaatggaggagctagagaaagtacccaaggagctaaagggatctgcaactctataggtggaacaacattatgagtttacggatccgaattctcgacggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgtgtgtgtgcgcgcacgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgaagcttgagctcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaaattcctgcagcccgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgatcgtatcgcgatcgctatcagatctataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaatcggatgccgggaccgacgagtgcagaggcgtgcaagcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - DNA, 13144 bp, synthetic construct
  source 1..13144: mol_type = other DNA; note = Expression vector with a Flag tag at the end of EGFP exon 2
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggagatcggtacttcgcgaatgcgtcgagatttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatgaattcccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgtcaggctggtgaaagggcaggcagcactttcccagcacactggccaaagccaacacggtagccgtccccctggcagtgacctgttatgatgacttcatcgccgtccagcaggaaggtcctggtctgcccctgctccacatcgatggcctttgttcccttccaggacagttccagcatggagccaaagctttcagggtctgatccactgatggttccagaagccaagaggtccccaggtctcaggttgcatccattaacagagtggtgtgtgagttgctgcagcatggtccagtacatgtgcttaaagttagacctgcagatggtagccgcctggctcattccttctcctttcaaagagacagacaggttgatatcaaatgtgtagggctggctgtggcagagatatggcaagggcttggggtcctgctttgggtttggcaccacaaagggcatgagggcatccataggcaccacccacggggagattgtggttccaaagcttttccccaggaatggcccaagtgggacgtactcccattgctggatgtctcgtgcgctccagtcgttcatgaggaccatcccgaaaatgtgttcatgggctttggaaatggggattggctctccgaatctgttcccagggcctacgaagaaagccatttccaactccatgtctaagagtctgcaggcaccatacacaggaggctttgagttatcaggtctcatctgccccatgggtcttcgaatcggggttccagataccacaatggaggaagctcggccatggtatcccacaggtaagtggagccaatttggcaacagcgcattctccttgcctctgaacataatgccaacattggtggcatgctgccgagaagagtagaagtccgtgtagtctcctatggtagcaggaaggtgcattgtcgcagaagcctgggaggtgaatgcacgctgccgaagctccttgtcatctctgagccgggcttggctggcagacagtaagttctgtaaggatgctcttgcctccttccatgcagcttgacccagacccatgaagttattgagagttgtctcatcgaagacatgttgatgtttggaaagggcaggtccggtaaagaggtgtttaatgacactcaggtccaggatctggtcaccgatggctacaccaatccgtggctttgggttgctttgagtggagaaaacaccatagggcaggttttggatgggaaagtcggagtcctcggccactggaataaaggacattgggccaggattctcctccacgtcaccgcatgttagaagacttcctctgccctctcctccggacttgtacagctcgtccatgccgccggtggagtggcggccctcggcgcgctcgtactgttccacgatggtgtagtcctcgttgtgggaggtgatgtccagcttgatgttgacgttgtaggcgccgggcagctgcacgggcttcttggccttgtaggtggtcttgacctcggcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcacctgaaatggaagaaaaaaacttcgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgtgaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagcaccctgcagcagggccgggaagcggccatctttccgctcacgcaactggtgccgaccgggccagccttgccgcccagggcggggcgatacacggcggcgcgaggccaggcaccagagcaggccatccgcggccccaaaaaccgaaaagaccttggcccgcatttacaagactatcttgccagaaaaaaagcgtcgcagcaggtcatcgtcgactggctagagagctcacttatcgaaagcagcgagacaggcgcgaaggtgcctctagaccagattcaccggtgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccatcgatacacggcggaccgtgaccacttaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccatggtcttgagctgaagaggacagcagtggagagcgcctctaacgggtgcggccgagcggaggacttttctcccgtagcctggcagccaggcgcctggagcactgcctgactaaaacccagatgccgagtggccgccggccccttagccctgcccctcgctcctggctaacatggccgaaaggtcgtattctccgggggaggacgggggcccggagaggagggggtggagtgcctggttttccagtcaggcggccggagggcagccctcaagaacggccctgaccgcccgcggggtgaggggccctttctgggcaggacccgccccttggtcccgcagagccttggtacttggacctgaaccttgctccgagagggagtcctcgcggacgtcagcgctgcggccgcgtatacgtttaaacggaacttaggaaattagtctgaacaggtgagagggtgcgccagagaacctgacagcttctggaacaggcggaagcacagaggcgctgaggcatcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctttgtgggccggggacagccagccaccatccggaccggaggacaggtgcccgcccggctggggaggcggcctaagccacagcagcagcggtcgccatcttggtccgagacccgccgaacttaggaaattagtctgaacaggtgagagggtgcgccagagaacctgacagcttctagaacaggccgaagcacagaggcgctgaggcagcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcgacctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctgtgtgggccggggacagccagccaccttcctgaccggaggacaggtgcccacccggcaggggaggcggcctaagccacagcagcagcggtcgccatcttggtcccgggactccaaggaacttaggaatttagtctgcttaggtgagagtctgtaccacctgggaactgccaaagcaacacagtgtctgagaaaggtcctgttttgggccttcttcttcgtccaggaggaggtccaaatacaagatatctgcgcaccttccctgtaagagagcttgccagcagagagtgctctgagcactgaaactcagaggagagaatctgtctcccaggtctgctgatagacggtaacagaatcaccagaagaacaatctctaaacagagtcaactataactactaactccagagattaccagtttaaacgtatacggccggccggcgcgccgccaccatggccaagggcaagcgccgcaacctgaccaaccgcaaccaggaccacagccccagccccgagcccagcacccccaccagccccagccccggcaaccccaacacccccgagaacctggacctggacctgaaggcctacctgatgatgatggtggaggacatcaagaaggacttcaacaagagcctgaaggagatccaggagaacaccgccaaggagctgcaggtgctgaaggagaagcaggagaacaccatcaagcaggtggaggtgctgaccgagaaggaggagaagacctacaagcaggtgatggagatgaacaagaccatcctggacctgaagcgcgaggtcgacaccatcaagaagacccagagcgaggccaccctggagatcgagaccctgggcaagaagagcggcaccatcgacctgagcatcagcaaccgcatccaggagatggaggagcgcatcagcggcgccgaggacagcatcgagaacatcggcaccaccatcaaggagaacggcaagtgcaagaagatcctgacccagaacatccaggagatccaggacaccatccgccgccccaacgtgcgcatcatcggcgtggacgagaacgaggacttccagctgaagggccccgccaacatcttcaacaagatcatcgaggagaacttccccaacctgaagaacgagatgcacatgaacatccaggaggcctaccgcacccccaaccgcctggaccagaagcgcaacagctctagacacatcatcatccgcaccagcaacgccctgaacaaggaccgcatcctgaaggccgtgcgcgagaagggccaggtgacctacaagggcaagcccatccgcatcacccccgacttcagccccgagaccatgaaggcccgccgcgcctggaccgacgtgatccagaccctgcgcgagcacaagctgcagccccgcctgctgtaccccgccaagctgagcatcatcatcgagggcgagaccaaggtgttccacgacaagaccaagttcacccactacctgagcaccaaccccgccctgcagcgcatcatcaccgagaagaaccagtacaagaacggcaacaacgccctggagaagacccgccgctaatccctcaacaaaccttaccggtgatacccacgatataagatacaattgcctaaacacatgaaactcaagaaaaatgaagactgaagtgtggacactatgcccctccttagaagtggggacaaaacagccatggaaggagttacagaaataaagtttggagctgagatgaaaggagggaccatgtagagactgccatatccagggttccaccccataatcagcatccaagctctgacaccattgcatatactaggaagattttatcgaaaggacccagatgtagctgtctcttgtgagactatgcggggcctagcaacacagaagtggatgctcacagtcagctaatggatggatcacagggctttcaatggaggagctagagaaagtacccaaggagctaaagggatctgcaactctataggtggaacaacattatgagtttacggatccgaattctcgacggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttatcgtcgtcatccttgtaatccttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgtgtgtgtgcgcgcacgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgaagcttgagctcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaaattcctgcagcccgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgatcgtatcgcgatcgctatcagatctataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaatcggatgccgggaccgacgagtgcagaggcgtgcaagcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 6 - DNA, 3612 bp, synthetic construct
  regulatory 1..180: note = HADHA/B promoter HADHA side; regulatory_class = promoter
  misc_feature 156: note = HADHA start
  exon 184..561: note = mCherry coding sequence (CDS), exon 1 (mCherry E1)
  intron 562..1417: note = EF1 intron, the modified intron of human eukaryotic translation elongation factor 1 alpha 1 (EEF1A1) gene
  exon 1418..1747: note = mCherry coding sequence (CDS), exon 2 (mCherry E2)
  misc_structure 1748..1810: note = T2A peptide coding element
  regulatory 3071..3298: note = bGH polyA polyadenylation signal; regulatory_class = polyA_signal_sequence
  misc_feature 3307..3612: note = piggyBac (PB) left inverted terminal repeat (ITR)
  source 1..3612: mol_type = other DNA; note = First expression unit (HADHA side) of the expression vector with full ORFeus and no amiR
  CDS 1811..3070: gene = fumarylacetoacetate hydrolase (Fah) coding sequence (CDS); transl_table = 1
gacctttcggccatgttagccaggagcgaggggcagggctaaggggccggcggccactcggcatctgggttttagtcaggcagtgctccaggcgcctggctgccaggctacgggagaaaagtcctccgctcggccgcacccgttagaggcgctctccactgctgtcctcttcagctcaagaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtaagtggtcacggtccgccgtgtatcgatggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcaccggtgaatctggtctagaggcaccttcgcgcctgtctcgctgctttcgataagtgagctctctagccagtcgacgatgacctgctgcgacgctttttttctggcaagatagtcttgtaaatgcgggccaaggtcttttcggtttttggggccgcggatggcctgctctggtgcctggcctcgcgccgccgtgtatcgccccgccctgggcggcaaggctggcccggtcggcaccagttgcgtgagcggaaagatggccgcttcccggccctgctgcagggtgctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttcacgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcgaagtttttttcttccatttcaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtccggaggagagggcagaggaagtcttctaacatgcggtgacgtggaggagaatcctggcccaatgtcctttattccagtggccgaggactccgactttcccatccaaaacctgccctatggtgttttctccactcaaagcaacccaaagccacggattggtgtagccatcggtgaccagatcctggacctgagtgtcattaaacacctctttaccggacctgccctttccaaacatcaacatgtcttcgatgagacaactctcaataacttcatgggtctgggtcaagctgcatggaaggaggcaagagcatccttacagaacttactgtctgccagccaagcccggctcagagatgacaaggagcttcggcagcgtgcattcacctcccaggcttctgcgacaatgcaccttcctgctaccataggagactacacggacttctactcttctcggcagcatgccaccaatgttggcattatgttcagaggcaaggagaatgcgctgttgccaaattggctccacttacctgtgggataccatggccgagcttcctccattgtggtatctggaaccccgattcgaagacccatggggcagatgagacctgataactcaaagcctcctgtgtatggtgcctgcagactcttagacatggagttggaaatggctttcttcgtaggccctgggaacagattcggagagccaatccccatttccaaagcccatgaacacattttcgggatggtcctcatgaacgactggagcgcacgagacatccagcaatgggagtacgtcccacttgggccattcctggggaaaagctttggaaccacaatctccccgtgggtggtgcctatggatgccctcatgccctttgtggtgccaaacccaaagcaggaccccaagcccttgccatatctctgccacagccagccctacacatttgatatcaacctgtctgtctctttgaaaggagaaggaatgagccaggcggctaccatctgcaggtctaactttaagcacatgtactggaccatgctgcagcaactcacacaccactctgttaatggatgcaacctgagacctggggacctcttggcttctggaaccatcagtggatcagaccctgaaagctttggctccatgctggaactgtcctggaagggaacaaaggccatcgatgtggagcaggggcagaccaggaccttcctgctggacggcgatgaagtcatcataacaggtcactgccagggggacggctaccgtgttggctttggccagtgtgctgggaaagtgctgcctgccctttcaccagcctgacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaattcatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctaggg

SEQ ID NO: 7 - DNA, 3945 bp, synthetic construct
  regulatory 1..180: note = HADHA/B promoter HADHA side; regulatory_class = promoter
  misc_feature 156: note = HADHA start
  exon 184..561: note = mCherry coding sequence (CDS), exon 1 (mCherry E1)
  intron 562..795: note = EF1 intron, the modified intron of human eukaryotic translation elongation factor 1 alpha 1 (EEF1A1) gene
  misc_feature 796..1142: note = inserted amiR-mp53/1 element
  intron 1143..1750: note = EF1 intron, the modified intron of human eukaryotic translation elongation factor 1 alpha 1 (EEF1A1) gene
  exon 1751..2080: note = mCherry coding sequence (CDS), exon 2 (mCherry E2)
  misc_structure 2081..2143: note = T2A peptide coding element
  regulatory 3404..3631: note = bGH polyA polyadenylation signal; regulatory_class = polyA_signal_sequence
  misc_feature 3640..3945: note = piggyBac (PB) left inverted terminal repeat (ITR)
  source 1..3945: mol_type = other DNA; note = First expression unit (HADHA side) with amiR-mP53 element
  CDS 2144..3403: gene = fumarylacetoacetate hydrolase (Fah) coding sequence (CDS); transl_table = 1
gacctttcggccatgttagccaggagcgaggggcagggctaaggggccggcggccactcggcatctgggttttagtcaggcagtgctccaggcgcctggctgccaggctacgggagaaaagtcctccgctcggccgcacccgttagaggcgctctccactgctgtcctcttcagctcaagaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtaagtggtcacggtccgccgtgtatcgatggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcaccggtgtgtgagctctgtttgaatgaggcttcagtactttacagaatcgttgcctgcacatcttggaaacacttgctgggattacttcttcaggttaacccaacagaaggctcgagaaggtatattgctgttgacagtgagcgatcactgcatggacgatctgtttagtgaagccacagatgtaaacagatcgtccatgcagtgagtgcctactgcctcggacttcaaggggctactttaggagcaattatcttgtttactaaaactgaataccttgctatctctttgatacatttttacaaagctgaattaaaatggtataaattaaatcactttgtcgacgtgttctagaggcaccttcgcgcctgtctcgctgctttcgataagtgagctctctagccagtcgacgatgacctgctgcgacgctttttttctggcaagatagtcttgtaaatgcgggccaaggtcttttcggtttttggggccgcggatggcctgctctggtgcctggcctcgcgccgccgtgtatcgccccgccctgggcggcaaggctggcccggtcggcaccagttgcgtgagcggaaagatggccgcttcccggccctgctgcagggtgctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttcacgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcgaagtttttttcttccatttcaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtccggaggagagggcagaggaagtcttctaacatgcggtgacgtggaggagaatcctggcccaatgtcctttattccagtggccgaggactccgactttcccatccaaaacctgccctatggtgttttctccactcaaagcaacccaaagccacggattggtgtagccatcggtgaccagatcctggacctgagtgtcattaaacacctctttaccggacctgccctttccaaacatcaacatgtcttcgatgagacaactctcaataacttcatgggtctgggtcaagctgcatggaaggaggcaagagcatccttacagaacttactgtctgccagccaagcccggctcagagatgacaaggagcttcggcagcgtgcattcacctcccaggcttctgcgacaatgcaccttcctgctaccataggagactacacggacttctactcttctcggcagcatgccaccaatgttggcattatgttcagaggcaaggagaatgcgctgttgccaaattggctccacttacctgtgggataccatggccgagcttcctccattgtggtatctggaaccccgattcgaagacccatggggcagatgagacctgataactcaaagcctcctgtgtatggtgcctgcagactcttagacatggagttggaaatggctttcttcgtaggccctgggaacagattcggagagccaatccccatttccaaagcccatgaacacattttcgggatggtcctcatgaacgactggagcgcacgagacatccagcaatgggagtacgtcccacttgggccattcctggggaaaagctttggaaccacaatctccccgtgggtggtgcctatggatgccctcatgccctttgtggtgccaaacccaaagcaggaccccaagcccttgccatatctctgccacagccagccctacacatttgatatcaacctgtctgtctctttgaaaggagaaggaatgagccaggcggctaccatctgcaggtctaactttaagcacatgtactggaccatgctgcagcaactcacacaccactctgttaatggatgcaacctgagacctggggacctcttggcttctggaaccatcagtggatcagaccctgaaagctttggctccatgctggaactgtcctggaagggaacaaaggccatcgatgtggagcaggggcagaccaggaccttcctgctggacggcgatgaagtcatcataacaggtcactgccagggggacggctaccgtgttggctttggccagtgtgctgggaaagtgctgcctgccctttcaccagcctgacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaattcatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctaggg

SEQ ID NO: 8 - DNA, 10666 bp, synthetic construct
  regulatory 1..210: note = HADHA/B promoter HADHB side; regulatory_class = promoter
  misc_feature 196: note = HADHB start
  misc_feature 235..1834: note = TF monomers
  3'UTR 7058..7487: note = 3' UTR (1st 430 bp)
  regulatory 7504..7727: note = hsvTK polyA polyadenylation signal (in reverse orientation); regulatory_class = polyA_signal_sequence
  exon 7731..7916: note = EGFP exon 2 (E2), in reverse orientation
  intron 7917..8818: note = hGamma Globin intron 2
  exon 8819..9352: note = EGFP exon 1 (E1), in reverse orientation
  regulatory 9402..9986: note = CMV promoter, in reverse orientation; regulatory_class = promoter
  regulatory 10017..10258: note = SV40 polyA polyadenylation signal; regulatory_class = polyA_signal_sequence
  misc_feature 10430..10666: note = piggyBac (PB) right inverted terminal repeat (ITR)
  source 1..10666: mol_type = other DNA; note = Second expression unit (HADHB side) of the expression vector with full ORFeus and no amiR
  CDS 2056..3171: gene = ORF1; transl_table = 1
  CDS 3212..7057: gene = ORF2; transl_table = 1
gtattctccgggggaggacgggggcccggagaggagggggtggagtgcctggttttccagtcaggcggccggagggcagccctcaagaacggccctgaccgcccgcggggtgaggggccctttctgggcaggacccgccccttggtcccgcagagccttggtacttggacctgaaccttgctccgagagggagtcctcgcggacgtcagcgctgcggccgcgtatacgtttaaacggaacttaggaaattagtctgaacaggtgagagggtgcgccagagaacctgacagcttctggaacaggcggaagcacagaggcgctgaggcatcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctttgtgggccggggacagccagccaccatccggaccggaggacaggtgcccgcccggctggggaggcggcctaagccacagcagcagcggtcgccatcttggtccgagacccgccgaacttaggaaattagtctgaacaggtgagagggtgcgccagagaacctgacagcttctagaacaggccgaagcacagaggcgctgaggcagcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcgacctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctgtgtgggccggggacagccagccaccttcctgaccggaggacaggtgcccacccggcaggggaggcggcctaagccacagcagcagcggtcgccatcttggtcccgggactccaaggaacttaggaatttagtctgcttaggtgagagtctgtaccacctgggaactgccaaagcaacacagtgtctgagaaaggtcctgttttgggccttcttcttcgtccaggaggaggtccaaatacaagatatctgcgcaccttccctgtaagagagcttgccagcagagagtgctctgagcactgaaactcagaggagagaatctgtctcccaggtctgctgatagacggtaacagaatcaccagaagaacaatctctaaacagagtcaactataactactaactccagagattaccagtttaaacgtatacggccggccggcgcgccgccaccatggccaagggcaagcgccgcaacctgaccaaccgcaaccaggaccacagccccagccccgagcccagcacccccaccagccccagccccggcaaccccaacacccccgagaacctggacctggacctgaaggcctacctgatgatgatggtggaggacatcaagaaggacttcaacaagagcctgaaggagatccaggagaacaccgccaaggagctgcaggtgctgaaggagaagcaggagaacaccatcaagcaggtggaggtgctgaccgagaaggaggagaagacctacaagcaggtgatggagatgaacaagaccatcctggacctgaagcgcgaggtcgacaccatcaagaagacccagagcgaggccaccctggagatcgagaccctgggcaagaagagcggcaccatcgacctgagcatcagcaaccgcatccaggagatggaggagcgcatcagcggcgccgaggacagcatcgagaacatcggcaccaccatcaaggagaacggcaagtgcaagaagatcctgacccagaacatccaggagatccaggacaccatccgccgccccaacgtgcgcatcatcggcgtggacgagaacgaggacttccagctgaagggccccgccaacatcttcaacaagatcatcgaggagaacttccccaacctgaagaacgagatgcacatgaacatccaggaggcctaccgcacccccaaccgcctggaccagaagcgcaacagctctagacacatcatcatccgcaccagcaacgccctgaacaaggaccgcatcctgaaggccgtgcgcgagaagggccaggtgacctacaagggcaagcccatccgcatcacccccgacttcagccccgagaccatgaaggcccgccgcgcctggaccgacgtgatccagaccctgcgcgagcacaagctgcagccccgcctgctgtaccccgccaagctgagcatcatcatcgagggcgagaccaaggtgttccacgacaagaccaagttcacccactacctgagcaccaaccccgccctgcagcgcatcatcaccgagaagaaccagtacaagaacggcaacaacgccctggagaagacccgccgctaatccctcaacaaaccttaattaagacagccacaagaacagaatgccccccctgaccaccaagatcaccggcagcaacaactacttcagcctgatcagcctgaacatcaacggcctgaacagccccatcaagcggcaccgcctgaccaactggctgcacaagcaggaccccaccttctgttgcctccaggagacccacctgcgcgagaaggaccggcactacctgcggatgaagggctggaagaccatctttcaggccaacggcatgaagaagcaggctggcgtggccatcctgatcagcgacaagatcgacttccagcccaaggtgatcaagaaggacaaggagggccacttcatcctgatcaagggcaagatcctgcaggaggagctgagcattctgaacatctacgcccccaacacccgcgccgccaccttcaccaaggagaccctcgtgaagctgaaggcccacatcgctccccacaccatcatcgtcggcgacttcaacacccccctgagccccatggacagatcttggaagcagaagctgaaccgcgacaccctgaagctgaccgaggtgatgaagcagatggacctgaccgacatctaccgcaccttctaccccaagaccaagggctacaccttcttcagcgctccccacggcaccttcagcaagatcgaccacatcatcggccacaagagcgggctgaaccggctgaagaacatcgagatcgtgccctgcatcctgagcgaccaccatgccctgcgcctgatcttcaacaacaagattaacaaccgcaagcccaccttcacctggaagctgaacaacaccctgctgaacgacaccctggtgaaggaaggcatcaagaaggagatcaaggacttcctggagttcaacgagaacgaggccaccacctaccctaacctgtgggacaccatgaaggccttcctgcggggcaagctgatcgccatgagcgccttcaagaagaagcgggagcgcgcccacactagtagcctgaccacccacctgaaggccctggagaagaaggaggctaactcccccaagcgctcccggcggcaggagatcatcaagctgcgcggcgagatcaaccaggtggagacccggcgcaccatccaacggatcaaccagacccggtcttggttcttcgagaagatcaacaagatcgacaagcccctggctcgcctgaccaagggccaccgcgacaagatcctgatcaacaagatccgcaacgagaagggcgacatcaccaccgaccccgaggagatccagaacaccatccgcagcttctacaagcgcctgtacagcaccaagctggagaacctggacgagatggacaagttcctggaccgctatcaggtgcccaagctgaaccaggaccaggtggacctgctgaacagccccatctcccccaaggaaatcgaggccgtgatcaacagcctgcccgccaagaagagccccggccccgacggcttcagcgctgagttctaccagaccttcaaggaggacctgacccccgtgctgcacaagcttttccacaagatcgaggtcgagggcatcctccccaacagcttctacgaggccaccatcaccctgatccccaagccccagaaggaccccaccaagatcgagaacttccgccccatcagcctgatgaacatcgacgccaagatcctgaacaagatcctcgccaaccgcatccaggagcacatcaaggccatcatccaccctgaccaggtcggcttcatccccggcatgcagggctggttcaacatccgcaagagcatcaacgtgatccactacatcaacaagctgaaggacaagaaccacatgatcatcagcctggacgccgagaaggccttcgacaagatccagcaccccttcatgatcaaggtcctggagcgcagcggcattcagggccagtacctcaacatgatcaaggccatctacagcaagcccgtcgccaacatcaaggtgaacggggagaagctggaggccatccctctgaagagcggcacgcgtcagggctgtcctctgtccccctacctgttcaacatcgtgctggaggtgctggcccgcgctatccggcagcagaaggagatcaagggcatccagatcggcaaggaggaagtgaagatcagcctgttcgccgacgacatgatcgtgtacatcagcgaccccaagaacagcaaccgcgagctcctgaacctgatcaacagcttcggcgaggtggctggctacaagatcaacagcaacaagagcatggccttcctgtacaccaagaacaagcaggccgagaaggagatccgcgagaccacccccttcagcatcgccaccaacaacatcaagtacctgggcgtgaccctgaccaaggaggtgaaggacctgtacgacaagaacttcaagagcctgaagaaggagatcaaggaggacctgcgccgctggaaggacctgccctgcagctggatcggccgcaccaacatcgtgaagatggccatcctgcccaaggctatctaccgcttcaacgccatccccatcaagatccccacccagttcttcaacgagctcgagggcgccatctgcaagttcatctggaacaacaagaagccccgcatcgccaagaccctgctgaaggacaagcgcaccagtggggggatcaccatgcccgacctgaagctgtactaccgcgccatcgtgatcaagaccgcctggtactggtaccgcgaccgccaggtggaccagtggaaccgcatcgaggaccccgagatgaacccccacacctacggccacctgatcttcgacaagggcgccaagaccatccagtggaagaaggacagcatcttcaacaactggtgctggcacaactggctgctgagctgccgccgcatgcgcatcgacccctacctaagcccctgcaccaaggtgaagagcaagtggatcaaggagctgcacatcaagcccgagactctgaagctgatcgaggagaaggtgggcaagagcctggaggacatgggcaccggcgaaaagttcctgaaccgcaccgccatggcctgtgccgtgcgcagccggatcgataagtgggacctgatgaagctgcagtccttctgtaaggccaaggataccgtgtacaagaccaagcgccccccgaccgactgggagcgcatcttcacctaccccaagagcgaccgcggcctgatcagcaacatctacaaggagctgaagaaggtggacctgcgcaagagcaacaacccgctgaagaagtggggctccgagctgaacaaggagttctcccccgaggagtaccgcatggccgagaagcacctgaagaagtgcagcaccagcctgatcatccgcgagatgcagatcaagaccaccctgcgcttccacctgacccccgtgcggatggccaagatcaagaacagcggcgactcgcgatgctggcggggctgcggcgagcgcggcaccctgctgcactgctggtgggactgtcgcctggtccagcccctgtggaagagcgtgtggcggttcctgcggaagctggacatcgtgctgcccgaggaccccgctatccccctgctgggcatctaccctgaggaggcccccaccggcaagaaggacacctgcagtaccatgttcatcgccgctctgttcatcatcgcccgcaactggaaggagccccgctgccccagcaccgaggagtggattcagaagatgtggtacatctacaccatggagtactacagcgccatcaagaagaacgagttcatgaagttcctggccaagtggatggacctggagagcatcatcctgagcgaggtgacccagagccagcgcaacagccacaacatgtacagcctgatcagcggctactagcctaaaaccggtgatacccacgatataagatacaattgcctaaacacatgaaactcaagaaaaatgaagactgaagtgtggacactatgcccctccttagaagtggggacaaaacagccatggaaggagttacagaaataaagtttggagctgagatgaaaggagggaccatgtagagactgccatatccagggttccaccccataatcagcatccaagctctgacaccattgcatatactaggaagattttatcgaaaggacccagatgtagctgtctcttgtgagactatgcggggcctagcaacacagaagtggatgctcacagtcagctaatggatggatcacagggctttcaatggaggagctagagaaagtacccaaggagctaaagggatctgcaactctataggtggaacaacattatgagtttacggatccgaattctcgacggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgtgtgtgtgcgcgcacgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgaagcttgagctcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaaattcctgcagcccgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgatcgtatcgcgatcgctatcagatctataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctaggg

SEQ ID NO: 9 - DNA, 6790 bp, synthetic construct
  regulatory 1..210: note = HADHA/B promoter HADHB side; regulatory_class = promoter
  misc_feature 196: note = HADHB start
  misc_feature 235..1834: note = TF monomers
  3'UTR 3189..3611: note = 3' UTR (1st 430 bp)
  regulatory 3628..3851: note = hsvTK polyA polyadenylation signal (in reverse orientation); regulatory_class = polyA_signal_sequence
  exon 3855..4040: note = EGFP exon 2 (E2), in reverse orientation
  intron 4041..4942: note = hGamma Globin intron 2
  exon 4943..5476: note = EGFP exon 1 (E1), in reverse orientation
  regulatory 5526..6110: note = CMV promoter, in reverse orientation; regulatory_class = promoter
  regulatory 6141..6382: note = SV40 polyA polyadenylation signal; regulatory_class = polyA_signal_sequence
  misc_feature 6554..6790: note = piggyBac (PB) right inverted terminal repeat (ITR)
  source 1..6790: mol_type = other DNA; note = Second expression unit (HADHB side) with ORF1 and TF monomers
  CDS 2056..3171: gene = ORF1; transl_table = 1
gtattctccgggggaggacgggggcccggagaggagggggtggagtgcctggttttccagtcaggcggccggagggcagccctcaagaacggccctgaccgcccgcggggtgaggggccctttctgggcaggacccgccccttggtcccgcagagccttggtacttggacctgaaccttgctccgagagggagtcctcgcggacgtcagcgctgcggccgcgtatacgtttaaacggaacttaggaaattagtctgaacaggtgagagggtgcgccagagaacctgacagcttctggaacaggcggaagcacagaggcgctgaggcatcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctttgtgggccggggacagccagccaccatccggaccggaggacaggtgcccgcccggctggggaggcggcctaagccacagcagcagcggtcgccatcttggtccgagacccgccgaacttaggaaattagtctgaacaggtgagagggtgcgccagagaacctgacagcttctagaacaggccgaagcacagaggcgctgaggcagcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcgacctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctgtgtgggccggggacagccagccaccttcctgaccggaggacaggtgcccacccggcaggggaggcggcctaagccacagcagcagcggtcgccatcttggtcccgggactccaaggaacttaggaatttagtctgcttaggtgagagtctgtaccacctgggaactgccaaagcaacacagtgtctgagaaaggtcctgttttgggccttcttcttcgtccaggaggaggtccaaatacaagatatctgcgcaccttccctgtaagagagcttgccagcagagagtgctctgagcactgaaactcagaggagagaatctgtctcccaggtctgctgatagacggtaacagaatcaccagaagaacaatctctaaacagagtcaactataactactaactccagagattaccagtttaaacgtatacggccggccggcgcgccgccaccatggccaagggcaagcgccgcaacctgaccaaccgcaaccaggaccacagccccagccccgagcccagcacccccaccagccccagccccggcaaccccaacacccccgagaacctggacctggacctgaaggcctacctgatgatgatggtggaggacatcaagaaggacttcaacaagagcctgaaggagatccaggagaacaccgccaaggagctgcaggtgctgaaggagaagcaggagaacaccatcaagcaggtggaggtgctgaccgagaaggaggagaagacctacaagcaggtgatggagatgaacaagaccatcctggacctgaagcgcgaggtcgacaccatcaagaagacccagagcgaggccaccctggagatcgagaccctgggcaagaagagcggcaccatcgacctgagcatcagcaaccgcatccaggagatggaggagcgcatcagcggcgccgaggacagcatcgagaacatcggcaccaccatcaaggagaacggcaagtgcaagaagatcctgacccagaacatccaggagatccaggacaccatccgccgccccaacgtgcgcatcatcggcgtggacgagaacgaggacttccagctgaagggccccgccaacatcttcaacaagatcatcgaggagaacttccccaacctgaagaacgagatgcacatgaacatccaggaggcctaccgcacccccaaccgcctggaccagaagcgcaacagctctagacacatcatcatccgcaccagcaacgccctgaacaaggaccgcatcctgaaggccgtgcgcgagaagggccaggtgacctacaagggcaagcccatccgcatcacccccgacttcagccccgagaccatgaaggcccgccgcgcctggaccgacgtgatccagaccctgcgcgagcacaagctgcagccccgcctgctgtaccccgccaagctgagcatcatcatcgagggcgagaccaaggtgttccacgacaagaccaagttcacccactacctgagcaccaaccccgccctgcagcgcatcatcaccgagaagaaccagtacaagaacggcaacaacgccctggagaagacccgccgctaatccctcaacaaaccttaccggtgatacccacgatataagatacaattgcctaaacacatgaaactcaagaaaaatgaagactgaagtgtggacactatgcccctccttagaagtggggacaaaacagccatggaaggagttacagaaataaagtttggagctgagatgaaaggagggaccatgtagagactgccatatccagggttccaccccataatcagcatccaagctctgacaccattgcatatactaggaagattttatcgaaaggacccagatgtagctgtctcttgtgagactatgcggggcctagcaacacagaagtggatgctcacagtcagctaatggatggatcacagggctttcaatggaggagctagagaaagtacccaaggagctaaagggatctgcaactctataggtggaacaacattatgagtttacggatccgaattctcgacggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgtgtgtgtgcgcgcacgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgaagcttgagctcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaaattcctgcagcccgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgatcgtatcgcgatcgctatcagatctataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctaggg

SEQ ID NO: 10 - DNA, 5014 bp, synthetic construct
  regulatory 1..210: note = HADHA/B promoter HADHB side; regulatory_class = promoter
  misc_feature 196: note = HADHB start
  3'UTR 1413..1835: note = 3' UTR (1st 430 bp)
  regulatory 1852..2075: note = hsvTK polyA polyadenylation signal (in reverse orientation); regulatory_class = polyA_signal_sequence
  exon 2079..2264: note = EGFP exon 2 (E2), in reverse orientation
  intron 2265..3166: note = hGamma Globin intron 2
  exon 3167..3700: note = EGFP exon 1 (E1), in reverse orientation
  regulatory 3750..4334: note = CMV promoter, in reverse orientation; regulatory_class = promoter
  regulatory 4365..4606: note = SV40 polyA polyadenylation signal; regulatory_class = polyA_signal_sequence
  misc_feature 4778..5014: note = piggyBac (PB) right inverted terminal repeat (ITR)
  source 1..5014: mol_type = other DNA; note = Second expression unit (HADHB side) with ORF1 and without TF monomers
  CDS 280..1395: gene = ORF1; transl_table = 1
gtattctccgggggaggacgggggcccggagaggagggggtggagtgcctggttttccagtcaggcggccggagggcagccctcaagaacggccctgaccgcccgcggggtgaggggccctttctgggcaggacccgccccttggtcccgcagagccttggtacttggacctgaaccttgctccgagagggagtcctcgcggacgtcagcgctgcggccgcgtgtggatccgtgtactagttatcgcggccgctatcggccggccggcgcgccgccaccatggccaagggcaagcgccgcaacctgaccaaccgcaaccaggaccacagccccagccccgagcccagcacccccaccagccccagccccggcaaccccaacacccccgagaacctggacctggacctgaaggcctacctgatgatgatggtggaggacatcaagaaggacttcaacaagagcctgaaggagatccaggagaacaccgccaaggagctgcaggtgctgaaggagaagcaggagaacaccatcaagcaggtggaggtgctgaccgagaaggaggagaagacctacaagcaggtgatggagatgaacaagaccatcctggacctgaagcgcgaggtcgacaccatcaagaagacccagagcgaggccaccctggagatcgagaccctgggcaagaagagcggcaccatcgacctgagcatcagcaaccgcatccaggagatggaggagcgcatcagcggcgccgaggacagcatcgagaacatcggcaccaccatcaaggagaacggcaagtgcaagaagatcctgacccagaacatccaggagatccaggacaccatccgccgccccaacgtgcgcatcatcggcgtggacgagaacgaggacttccagctgaagggccccgccaacatcttcaacaagatcatcgaggagaacttccccaacctgaagaacgagatgcacatgaacatccaggaggcctaccgcacccccaaccgcctggaccagaagcgcaacagctctagacacatcatcatccgcaccagcaacgccctgaacaaggaccgcatcctgaaggccgtgcgcgagaagggccaggtgacctacaagggcaagcccatccgcatcacccccgacttcagccccgagaccatgaaggcccgccgcgcctggaccgacgtgatccagaccctgcgcgagcacaagctgcagccccgcctgctgtaccccgccaagctgagcatcatcatcgagggcgagaccaaggtgttccacgacaagaccaagttcacccactacctgagcaccaaccccgccctgcagcgcatcatcaccgagaagaaccagtacaagaacggcaacaacgccctggagaagacccgccgctaatccctcaacaaaccttaccggtgatacccacgatataagatacaattgcctaaacacatgaaactcaagaaaaatgaagactgaagtgtggacactatgcccctccttagaagtggggacaaaacagccatggaaggagttacagaaataaagtttggagctgagatgaaaggagggaccatgtagagactgccatatccagggttccaccccataatcagcatccaagctctgacaccattgcatatactaggaagattttatcgaaaggacccagatgtagctgtctcttgtgagactatgcggggcctagcaacacagaagtggatgctcacagtcagctaatggatggatcacagggctttcaatggaggagctagagaaagtacccaaggagctaaagggatctgcaactctataggtggaacaacattatgagtttacggatccgaattctcgacggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgtgtgtgtgcgcgcacgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgaagcttgagctcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaaattcctgcagcccgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgatcgtatcgcgatcgctatcagatctataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctaggg

SEQ ID NO: 11 - DNA, 6814 bp, synthetic construct
  regulatory 1..210: note = HADHA/B promoter HADHB side; regulatory_class = promoter
  misc_feature 196: note = HADHB start
  misc_feature 235..1834: note = TF monomers
  3'UTR 3189..3611: note = 3' UTR (1st 430 bp)
  regulatory 3628..3851: note = hsvTK polyA polyadenylation signal (in reverse orientation); regulatory_class = polyA_signal_sequence
  exon 3855..4064: note = EGFP exon 2 (E2), in reverse orientation
  exon 3858..3881: note = FLAG epitope tag (in reverse orientation)
  intron 4065..4966: note = hGamma Globin intron 2
  exon 4967..5500: note = EGFP exon 1 (E1), in reverse orientation
  regulatory 5550..6134: note = CMV promoter, in reverse orientation; regulatory_class = promoter
  regulatory 6165..6406: note = SV40 polyA polyadenylation signal; regulatory_class = polyA_signal_sequence
  misc_feature 6578..6814: note = piggyBac (PB) right inverted terminal repeat (ITR)
  source 1..6814: mol_type = other DNA; note = Second expression unit (HADHB side) with a Flag tag at the end of EGFP exon 2
  CDS 2056..3171: gene = ORF1; transl_table = 1
gtattctccgggggaggacgggggcccggagaggagggggtggagtgcctggttttccagtcaggcggccggagggcagccctcaagaacggccctgaccgcccgcggggtgaggggccctttctgggcaggacccgccccttggtcccgcagagccttggtacttggacctgaaccttgctccgagagggagtcctcgcggacgtcagcgctgcggccgcgtatacgtttaaacggaacttaggaaattagtctgaacaggtgagagggtgcgccagagaacctgacagcttctggaacaggcggaagcacagaggcgctgaggcatcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctttgtgggccggggacagccagccaccatccggaccggaggacaggtgcccgcccggctggggaggcggcctaagccacagcagcagcggtcgccatcttggtccgagacccgccgaacttaggaaattagtctgaacaggtgagagggtgcgccagagaacctgacagcttctagaacaggccgaagcacagaggcgctgaggcagcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcgacctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctgtgtgggccggggacagccagccaccttcctgaccggaggacaggtgcccacccggcaggggaggcggcctaagccacagcagcagcggtcgccatcttggtcccgggactccaaggaacttaggaatttagtctgcttaggtgagagtctgtaccacctgggaactgccaaagcaacacagtgtctgagaaaggtcctgttttgggccttcttcttcgtccaggaggaggtccaaatacaagatatctgcgcaccttccctgtaagagagcttgccagcagagagtgctctgagcactgaaactcagaggagagaatctgtctcccaggtctgctgatagacggtaacagaatcaccagaagaacaatctctaaacagagtcaactataactactaactccagagattaccagtttaaacgtatacggccggccggcgcgccgccaccatggccaagggcaagcgccgcaacctgaccaaccgcaaccaggaccacagccccagccccgagcccagcacccccaccagccccagccccggcaaccccaacacccccgagaacctggacctggacctgaaggcctacctgatgatgatggtggaggacatcaagaaggacttcaacaagagcctgaaggagatccaggagaacaccgccaaggagctgcaggtgctgaaggagaagcaggagaacaccatcaagcaggtggaggtgctgaccgagaaggaggagaagacctacaagcaggtgatggagatgaacaagaccatcctggacctgaagcgcgaggtcgacaccatcaagaagacccagagcgaggccaccctggagatcgagaccctgggcaagaagagcggcaccatcgacctgagcatcagcaaccgcatccaggagatggaggagcgcatcagcggcgccgaggacagcatcgagaacatcggcaccaccatcaaggagaacggcaagtgcaagaagatcctgacccagaacatccaggagatccaggacaccatccgccgccccaacgtgcgcatcatcggcgtggacgagaacgaggacttccagctgaagggccccgccaacatcttcaacaagatcatcgaggagaacttccccaacctgaagaacgagatgcacatgaacatccaggaggcctaccgcacccccaaccgcctggaccagaagcgcaacagctctagacacatcatcatccgcaccagcaacgccctgaacaaggaccgcatcctgaaggccgtgcgcgagaagggccaggtgacctacaagggcaagcccatccgcatcacccccgacttcagccccgagaccatgaaggcccgccgcgcctggaccgacgtgatccagaccctgcgcgagcacaagctgcagccccgcctgctgtaccccgccaagctgagcatcatcatcgagggcgagaccaaggtgttccacgacaagaccaagttcacccactacctgagcaccaaccccgccctgcagcgcatcatcaccgagaagaaccagtacaagaacggcaacaacgccctggagaagacccgccgctaatccctcaacaaaccttaccggtgatacccacgatataagatacaattgcctaaacacatgaaactcaagaaaaatgaagactgaagtgtggacactatgcccctccttagaagtggggacaaaacagccatggaaggagttacagaaataaagtttggagctgagatgaaaggagggaccatgtagagactgccatatccagggttccaccccataatcagcatccaagctctgacaccattgcatatactaggaagattttatcgaaaggacccagatgtagctgtctcttgtgagactatgcggggcctagcaacacagaagtggatgctcacagtcagctaatggatggatcacagggctttcaatggaggagctagagaaagtacccaaggagctaaagggatctgcaactctataggtggaacaacattatgagtttacggatccgaattctcgacggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttatcgtcgtcatccttgtaatccttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgtgtgtgtgcgcgcacgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgaagcttgagctcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaaattcctgcagcccgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgatcgtatcgcgatcgctatcagatctataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctaggg

SEQ ID NO: 12 - DNA, 2483 bp, synthetic construct
  regulatory 1..585: note = CMV promoter; regulatory_class = promoter
  intron 1169..2070: note = hGamma Globin intron 2
  regulatory 2260..2483: note = hsvTK polyA polyadenylation signal; regulatory_class = polyA_signal_sequence
  source 1..2483: mol_type = other DNA; note = EGFP-expressing element in the second expression unit (HADHB side)
  CDS 635..1168: note = EGFP exon 1 (E1); transl_table = 1
  CDS 2071..2256: note = EGFP exon 2 (E2); transl_table = 1
ttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctgttgagatgaaaggagacaataaagatgaacccatagtgagctgagagctccagcctggcctccagataactacacacccagcttccacccagaatcaagcctatgttaacttccctcaaagcctgagattttgctttcccattaaatgcaggtagttgttcttcttgcagcactagtcactggccataatttaaatcttgttatcttcttgccaccatgaaccctgtatgctgtaggctgaaaacgttaaaagaaacacacgctctcacacacacacaaacacacgtgcgcgcacacacacacacacacacacacacacacacacacagagctgactttcaaaatctactccagcccaaatgtttcaattgttcctcacccctggacatactttgcccccatctggaattaaaggatataagtttgtaatgaagcattagcagcattttatatgtgtccagctgatataggaatagccttagcaatgtatgtttggccaccaaagttccccactttgactgagccaatatatgccttctgcctgcatctttttaatgaccatacttgtcctgcctccagatagatgttttaaaacgaataacaaaaataggggaaagatgaaagttctttctaccgaaatctaataaagaaaagtcattttcctcatttccacctctcttttctcaaagtcaaagttgtccatctagattttcagaggcattccttaggccctaaaacattgccactgggtctcagcccagttagtcctctgcagtttcttcacccccaaccccagtatcttcaaacagctcacaccctgctgtgctcagatcaatactcagttgtctaagttgcctcgagactaaaggcaacagtgctgaaacatctcctggactcacctggccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcggcaataaaaagacagaataaaacgcacgggtgttgggtcgtttgttcggatcgatccgtcga

SEQ ID NO: 13 - protein, 178 aa, synthetic construct
  source 1..178: note = translation of EGFP exon 1 (E1) CDS from SEQ ID NO 12
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQ

SEQ ID NO: 14 - protein, 61 aa, synthetic construct
  source 1..61: note = translation of EGFP exon 2 (E2) CDS from SEQ ID NO 12
LADHYQQNTPIGDGPVLLPDNHYLSTQSALSKDPNEKRDHMVLLEFVTAAGITLGMDELYK

SEQ ID NO: 15 - DNA, 1260 bp, synthetic construct
  source 1..1260: mol_type = other DNA; note = fumarylacetoacetate hydrolase (Fah) coding sequence (CDS)
atgtcctttattccagtggccgaggactccgactttcccatccaaaacctgccctatggtgttttctccactcaaagcaacccaaagccacggattggtgtagccatcggtgaccagatcctggacctgagtgtcattaaacacctctttaccggacctgccctttccaaacatcaacatgtcttcgatgagacaactctcaataacttcatgggtctgggtcaagctgcatggaaggaggcaagagcatccttacagaacttactgtctgccagccaagcccggctcagagatgacaaggagcttcggcagcgtgcattcacctcccaggcttctgcgacaatgcaccttcctgctaccataggagactacacggacttctactcttctcggcagcatgccaccaatgttggcattatgttcagaggcaaggagaatgcgctgttgccaaattggctccacttacctgtgggataccatggccgagcttcctccattgtggtatctggaaccccgattcgaagacccatggggcagatgagacctgataactcaaagcctcctgtgtatggtgcctgcagactcttagacatggagttggaaatggctttcttcgtaggccctgggaacagattcggagagccaatccccatttccaaagcccatgaacacattttcgggatggtcctcatgaacgactggagcgcacgagacatccagcaatgggagtacgtcccacttgggccattcctggggaaaagctttggaaccacaatctccccgtgggtggtgcctatggatgccctcatgccctttgtggtgccaaacccaaagcaggaccccaagcccttgccatatctctgccacagccagccctacacatttgatatcaacctgtctgtctctttgaaaggagaaggaatgagccaggcggctaccatctgcaggtctaactttaagcacatgtactggaccatgctgcagcaactcacacaccactctgttaatggatgcaacctgagacctggggacctcttggcttctggaaccatcagtggatcagaccctgaaagctttggctccatgctggaactgtcctggaagggaacaaaggccatcgatgtggagcaggggcagaccaggaccttcctgctggacggcgatgaagtcatcataacaggtcactgccagggggacggctaccgtgttggctttggccagtgtgctgggaaagtgctgcctgccctttcaccagcctga

SEQ ID NO: 16 - protein, 419 aa, synthetic construct
  source 1..419: note = Fah protein, translation of Fah CDS from SEQ ID NOs 6-7 and 15
MSFIPVAEDSDFPIQNLPYGVFSTQSNPKPRIGVAIGDQILDLSVIKHLFTGPALSKHQHVFDETTLNNFMGLGQAAWKEARASLQNLLSASQARLRDDKELRQRAFTSQASATMHLPATIGDYTDFYSSRQHATNVGIMFRGKENALLPNWLHLPVGYHGRASSIVVSGTPIRRPMGQMRPDNSKPPVYGACRLLDMELEMAFFVGPGNRFGEPIPISKAHEHIFGMVLMNDWSARDIQQWEYVPLGPFLGKSFGTTISPWVVPMDALMPFVVPNPKQDPKPLPYLCHSQPYTFDINLSVSLKGEGMSQAATICRSNFKHMYWTMLQQLTHHSVNGCNLRPGDLLASGTISGSDPESFGSMLELSWKGTKAIDVEQGQTRTFLLDGDEVIITGHCQGDGYRVGFGQCAGKVLPALSPA

SEQ ID NO: 17 - DNA, 390 bp, synthetic construct
  source 1..390: mol_type = other DNA; note = HADHA/B promoter
cttgagctgaagaggacagcagtggagagcgcctctaacgggtgcggccgagcggaggacttttctcccgtagcctggcagccaggcgcctggagcactgcctgactaaaacccagatgccgagtggccgccggccccttagccctgcccctcgctcctggctaacatggccgaaaggtcgtattctccgggggaggacgggggcccggagaggagggggtggagtgcctggttttccagtcaggcggccggagggcagccctcaagaacggccctgaccgcccgcggggtgaggggccctttctgggcaggacccgccccttggtcccgcagagccttggtacttggacctgaaccttgctccgagagggagtcctcgcggacgtcagc

SEQ ID NO: 18 - DNA, 856 bp, synthetic construct
  source 1..856: mol_type = other DNA; note = EF1 intron, the modified intron of human eukaryotic translation elongation factor 1 alpha 1 (EEF1A1) gene
gtaagtggtcacggtccgccgtgtatcgatggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcaccggtgaatctggtctagaggcaccttcgcgcctgtctcgctgctttcgataagtgagctctctagccagtcgacgatgacctgctgcgacgctttttttctggcaagatagtcttgtaaatgcgggccaaggtcttttcggtttttggggccgcggatggcctgctctggtgcctggcctcgcgccgccgtgtatcgccccgccctgggcggcaaggctggcccggtcggcaccagttgcgtgagcggaaagatggccgcttcccggccctgctgcagggtgctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttcacgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcgaagtttttttcttccatttcag

SEQ ID NO: 19 - DNA, 373 bp, synthetic construct
  misc_feature 27..121: note = Endogenous miR30a 5' flanking
  misc_feature 155..176: note = Target with N1 A mismatch
  stem_loop 177..195: note = loop
  misc_feature 196..217: note = guide
  misc_feature 238..347: note = Endogenous miR30a 3' flanking
  source 1..373: mol_type = other DNA; note = amiR-mP53/1 element
gggcccgtgtaccggtgtgtgagctctgtttgaatgaggcttcagtactttacagaatcgttgcctgcacatcttggaaacacttgctgggattacttcttcaggttaacccaacagaaggctcgagaaggtatattgctgttgacagtgagcgatcactgcatggacgatctgtttagtgaagccacagatgtaaacagatcgtccatgcagtgagtgcctactgcctcggacttcaaggggctactttaggagcaattatcttgtttactaaaactgaataccttgctatctctttgatacatttttacaaagctgaattaaaatggtataaattaaatcactttgtcgacgtgttctagagtgtgccggc

SEQ ID NO: 20 - DNA, 1116 bp, synthetic construct
  source 1..1116: mol_type = other DNA; note = ORF1 CDS (same as in SEQ ID NOs 8-11)
atggccaagggcaagcgccgcaacctgaccaaccgcaaccaggaccacagccccagccccgagcccagcacccccaccagccccagccccggcaaccccaacacccccgagaacctggacctggacctgaaggcctacctgatgatgatggtggaggacatcaagaaggacttcaacaagagcctgaaggagatccaggagaacaccgccaaggagctgcaggtgctgaaggagaagcaggagaacaccatcaagcaggtggaggtgctgaccgagaaggaggagaagacctacaagcaggtgatggagatgaacaagaccatcctggacctgaagcgcgaggtcgacaccatcaagaagacccagagcgaggccaccctggagatcgagaccctgggcaagaagagcggcaccatcgacctgagcatcagcaaccgcatccaggagatggaggagcgcatcagcggcgccgaggacagcatcgagaacatcggcaccaccatcaaggagaacggcaagtgcaagaagatcctgacccagaacatccaggagatccaggacaccatccgccgccccaacgtgcgcatcatcggcgtggacgagaacgaggacttccagctgaagggccccgccaacatcttcaacaagatcatcgaggagaacttccccaacctgaagaacgagatgcacatgaacatccaggaggcctaccgcacccccaaccgcctggaccagaagcgcaacagctctagacacatcatcatccgcaccagcaacgccctgaacaaggaccgcatcctgaaggccgtgcgcgagaagggccaggtgacctacaagggcaagcccatccgcatcacccccgacttcagccccgagaccatgaaggcccgccgcgcctggaccgacgtgatccagaccctgcgcgagcacaagctgcagccccgcctgctgtaccccgccaagctgagcatcatcatcgagggcgagaccaaggtgttccacgacaagaccaagttcacccactacctgagcaccaaccccgccctgcagcgcatcatcaccgagaagaaccagtacaagaacggcaacaacgccctggagaagacccgccgctaa

SEQ ID NO: 21 - protein, 371 aa, synthetic construct
  source 1..371: note = ORF1 protein, translation of ORF1 CDS from SEQ ID NOs 8-11
MAKGKRRNLTNRNQDHSPSPEPSTPTSPSPGNPNTPENLDLDLKAYLMMMVEDIKKDFNKSLKEIQENTAKELQVLKEKQENTIKQVEVLTEKEEKTYKQVMEMNKTILDLKREVDTIKKTQSEATLEIETLGKKSGTIDLSISNRIQEMEERISGAEDSIENIGTTIKENGKCKKILTQNIQEIQDTIRRPNVRIIGVDENEDFQLKGPANIFNKIIEENFPNLKNEMHMNIQEAYRTPNRLDQKRNSSRHIIIRTSNALNKDRILKAVREKGQVTYKGKPIRITPDFSPETMKARRAWTDVIQTLREHKLQPRLLYPAKLSIIIEGETKVFHDKTKFTHYLSTNPALQRIITEKNQYKNGNNALEKTRR

SEQ ID NO: 22 - DNA, 3846 bp, synthetic construct
  source 1..3846: mol_type = other DNA; note = ORF2 CDS (same as in SEQ ID NO 8)
atgccccccctgaccaccaagatcaccggcagcaacaactacttcagcctgatcagcctgaacatcaacggcctgaacagccccatcaagcggcaccgcctgaccaactggctgcacaagcaggaccccaccttctgttgcctccaggagacccacctgcgcgagaaggaccggcactacctgcggatgaagggctggaagaccatctttcaggccaacggcatgaagaagcaggctggcgtggccatcctgatcagcgacaagatcgacttccagcccaaggtgatcaagaaggacaaggagggccacttcatcctgatcaagggcaagatcctgcaggaggagctgagcattctgaacatctacgcccccaacacccgcgccgccaccttcaccaaggagaccctcgtgaagctgaaggcccacatcgctccccacaccatcatcgtcggcgacttcaacacccccctgagccccatggacagatcttggaagcagaagctgaaccgcgacaccctgaagctgaccgaggtgatgaagcagatggacctgaccgacatctaccgcaccttctaccccaagaccaagggctacaccttcttcagcgctccccacggcaccttcagcaagatcgaccacatcatcggccacaagagcgggctgaaccggctgaagaacatcgagatcgtgccctgcatcctgagcgaccaccatgccctgcgcctgatcttcaacaacaagattaacaaccgcaagcccaccttcacctggaagctgaacaacaccctgctgaacgacaccctggtgaaggaaggcatcaagaaggagatcaaggacttcctggagttcaacgagaacgaggccaccacctaccctaacctgtgggacaccatgaaggccttcctgcggggcaagctgatcgccatgagcgccttcaagaagaagcgggagcgcgcccacactagtagcctgaccacccacctgaaggccctggagaagaaggaggctaactcccccaagcgctcccggcggcaggagatcatcaagctgcgcggcgagatcaaccaggtggagacccggcgcaccatccaacggatcaaccagacccggtcttggttcttcgagaagatcaacaagatcgacaagcccctggctcgcctgaccaagggccaccgcgacaagatcctgatcaacaagatccgcaacgagaagggcgacatcaccaccgaccccgaggagatccagaacaccatccgcagcttctacaagcgcctgtacagcaccaagctggagaacctggacgagatggacaagttcctggaccgctatcaggtgcccaagctgaaccaggaccaggtggacctgctgaacagccccatctcccccaaggaaatcgaggccgtgatcaacagcctgcccgccaagaagagccccggccccgacggcttcagcgctgagttctaccagaccttcaaggaggacctgacccccgtgctgcacaagcttttccacaagatcgaggtcgagggcatcctccccaacagcttctacgaggccaccatcaccctgatccccaagccccagaaggaccccaccaagatcgagaacttccgccccatcagcctgatgaacatcgacgccaagatcctgaacaagatcctcgccaaccgcatccaggagcacatcaaggccatcatccaccctgaccaggtcggcttcatccccggcatgcagggctggttcaacatccgcaagagcatcaacgtgatccactacatcaacaagctgaaggacaagaaccacatgatcatcagcctggacgccgagaaggccttcgacaagatccagcaccccttcatgatcaaggtcctggagcgcagcggcattcagggccagtacctcaacatgatcaaggccatctacagcaagcccgtcgccaacatcaaggtgaacggggagaagctggaggccatccctctgaagagcggcacgcgtcagggctgtcctctgtccccctacctgttcaacatcgtgctggaggtgctggcccgcgctatccggcagcagaaggagatcaagggcatccagatcggcaaggaggaagtgaagatcagcctgttcgccgacgacatgatcgtgtacatcagcgaccccaagaacagcaaccgcgagctcctgaacctgatcaacagcttcggcgaggtggctggctacaagatcaacagcaacaagagcatggccttcctgtacaccaagaacaagcaggccgagaaggagatccgcgagaccacccccttcagcatcgccaccaacaacatcaagtacctgggcgtgaccctgaccaaggaggtgaaggacctgtacgacaagaacttcaagagcctgaagaaggagatcaaggaggacctgcgccgctggaaggacctgccctgcagctggatcggccgcaccaacatcgtgaagatggccatcctgcccaaggctatctaccgcttcaacgccatccccatcaagatccccacccagttcttcaacgagctcgagggcgccatctgcaagttcatctggaacaacaagaagccccgcatcgccaagaccctgctgaaggacaagcgcaccagtggggggatcaccatgcccgacctgaagctgtactaccgcgccatcgtgatcaagaccgcctggtactggtaccgcgaccgccaggtggaccagtggaaccgcatcgaggaccccgagatgaacccccacacctacggccacctgatcttcgacaagggcgccaagaccatccagtggaagaaggacagcatcttcaacaactggtgctggcacaactggctgctgagctgccgccgcatgcgcatcgacccctacctaagcccctgcaccaaggtgaagagcaagtggatcaaggagctgcacatcaagcccgagactctgaagctgatcgaggagaaggtgggcaagagcctggaggacatgggcaccggcgaaaagttcctgaaccgcaccgccatggcctgtgccgtgcgcagccggatcgataagtgggacctgatgaagctgcagtccttctgtaaggccaaggataccgtgtacaagaccaagcgccccccgaccgactgggagcgcatcttcacctaccccaagagcgaccgcggcctgatcagcaacatctacaaggagctgaagaaggtggacctgcgcaagagcaacaacccgctgaagaagtggggctccgagctgaacaaggagttctcccccgaggagtaccgcatggccgagaagcacctgaagaagtgcagcaccagcctgatcatccgcgagatgcagatcaagaccaccctgcgcttccacctgacccccgtgcggatggccaagatcaagaacagcggcgactcgcgatgctggcggggctgcggcgagcgcggcaccctgctgcactgctggtgggactgtcgcctggtccagcccctgtggaagagcgtgtggcggttcctgcggaagctggacatcgtgctgcccgaggaccccgctatccccctgctgggcatctaccctgaggaggcccccaccggcaagaaggacacctgcagtaccatgttcatcgccgctctgttcatcatcgcccgcaactggaaggagccccgctgccccagcaccgaggagtggattcagaagatgtggtacatctacaccatggagtactacagcgccatcaagaagaacgagttcatgaagttcctggccaagtggatggacctggagagcatcatcctgagcgaggtgacccagagccagcgcaacagccacaacatgtacagcctgatcagcggctactag

SEQ ID NO: 23 - protein, 1281 aa, synthetic construct
  source 1..1281: note = ORF2 protein, translation of ORF2 CDS from SEQ ID NO: 8
MPPLTTKITGSNNYFSLISLNINGLNSPIKRHRLTNWLHKQDPTFCCLQETHLREKDRHYLRMKGWKTIFQANGMKKQAGVAILISDKIDFQPKVIKKDKEGHFILIKGKILQEELSILNIYAPNTRAATFTKETLVKLKAHIAPHTIIVGDFNTPLSPMDRSWKQKLNRDTLKLTEVMKQMDLTDIYRTFYPKTKGYTFFSAPHGTFSKIDHIIGHKSGLNRLKNIEIVPCILSDHHALRLIFNNKINNRKPTFTWKLNNTLLNDTLVKEGIKKEIKDFLEFNENEATTYPNLWDTMKAFLRGKLIAMSAFKKKRERAHTSSLTTHLKALEKKEANSPKRSRRQEIIKLRGEINQVETRRTIQRINQTRSWFFEKINKIDKPLARLTKGHRDKILINKIRNEKGDITTDPEEIQNTIRSFYKRLYSTKLENLDEMDKFLDRYQVPKLNQDQVDLLNSPISPKEIEAVINSLPAKKSPGPDGFSAEFYQTFKEDLTPVLHKLFHKIEVEGILPNSFYEATITLIPKPQKDPTKIENFRPISLMNIDAKILNKILANRIQEHIKAIIHPDQVGFIPGMQGWFNIRKSINVIHYINKLKDKNHMIISLDAEKAFDKIQHPFMIKVLERSGIQGQYLNMIKAIYSKPVANIKVNGEKLEAIPLKSGTRQGCPLSPYLFNIVLEVLARAIRQQKEIKGIQIGKEEVKISLFADDMIVYISDPKNSNRELLNLINSFGEVAGYKINSNKSMAFLYTKNKQAEKEIRETTPFSIATNNIKYLGVTLTKEVKDLYDKNFKSLKKEIKEDLRRWKDLPCSWIGRTNIVKMAILPKAIYRFNAIPIKIPTQFFNELEGAICKFIWNNKKPRIAKTLLKDKRTSGGITMPDLKLYYRAIVIKTAWYWYRDRQVDQWNRIEDPEMNPHTYGHLIFDKGAKTIQWKKDSIFNNWCWHNWLLSCRRMRIDPYLSPCTKVKSKWIKELHIKPETLKLIEEKVGKSLEDMGTGEKFLNRTAMACAVRSRIDKWDLMKLQSFCKAKDTVYKTKRPPTDWERIFTYPKSDRGLISNIYKELKKVDLRKSNNPLKKWGSELNKEFSPEEYRMAEKHLKKCSTSLIIREMQIKTTLRFHLTPVRMAKIKNSGDSRCWRGCGERGTLLHCWWDCRLVQPLWKSVWRFLRKLDIVLPEDPAIPLLGIYPEEAPTGKKDTCSTMFIAALFIIARNWKEPRCPSTEEWIQKMWYIYTMEYYSAIKKNEFMKFLAKWMDLESIILSEVTQSQRNSHNMYSLISGY

SEQ ID NO: 24 - DNA, 1600 bp, synthetic construct
  source 1..1600: mol_type = other DNA; note = TF monomers (same as in SEQ ID NOs 8-9 and 11)
cggaacttaggaaattagtctgaacaggtgagagggtgcgccagagaacctgacagcttctggaacaggcggaagcacagaggcgctgaggcatcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctttgtgggccggggacagccagccaccatccggaccggaggacaggtgcccgcccggctggggaggcggcctaagccacagcagcagcggtcgccatcttggtccgagacccgccgaacttaggaaattagtctgaacaggtgagagggtgcgccagagaacctgacagcttctagaacaggccgaagcacagaggcgctgaggcagcaccctgtgtgggccggggacagccggccaccttctggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcggcctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccagaggacaggtgcccgcccggctggggaggcgacctaagccacagcagcagcggtcgccatcttggtccgggacccgccgaacttaggaaattagtctgaacaggtgagagggtgcgccagagaacctgacagcttctggaacaggcggaagcacagaggcgctgaggcagcaccctgtgtgggccggggacagccagccaccttcctgaccggaggacaggtgcccacccggcaggggaggcggcctaagccacagcagcagcggtcgccatcttggtcccgggactccaaggaacttaggaatttagtctgcttaggtgagagtctgtaccacctgggaactgccaaagcaacacagtgtctgagaaaggtcctgttttgggccttcttcttcgtccaggagg

SEQ ID NO: 25 - DNA, 21 bp, synthetic construct
  source 1..21: mol_type = other DNA; note = Olfr16 forward primer
gagttcgtcttcctgggattc

SEQ ID NO: 26 - DNA, 20 bp, synthetic construct
  source 1..20: mol_type = other DNA; note = Olfr16 reverse primer
taatgatgttgccagccaga

SEQ ID NO: 27 - DNA, 23 bp, synthetic construct
  source 1..23: mol_type = other DNA; note = GFP forward primer
aagcagaagaacggcatcaaggt

SEQ ID NO: 28 - DNA, 21 bp, synthetic construct
  source 1..21: mol_type = other DNA; note = GFP-EJ reverse primer
tggtagtggtcggccagctgc

SEQ ID NO: 29 - protein, 8 aa, synthetic construct
  source 1..8: note = Flag-tag
DYKDDDDK

SEQ ID NO: 30 - protein, 14 aa, synthetic construct
  source 1..14: note = V5-tag
GKPIPNPLLGLDST

(19) United States
(12) Patent Application Publication
(10) Pub. No.: US 2025/0223610 A1
(43) Pub. Date: Jul. 10, 2025

(54) MEASUREMENT OF SOMATIC L1 RETROTRANSPOSITION ACTIVITY

(71) Applicant: SZEGEDI BIOLÓGIAI KUTATÓKÖZPONT, Szeged (HU)

(72) Inventors: Lajos MÁTÉS, Szeged (HU); Réka KARKAS, Szeged (HU); Andrea MÁTÉS-NAGY, Szeged (HU); Gergely IMRE, Békéscsaba (HU); Khaldoon Sadiq Ahmed ABDULLAH, Szeged (HU); Anna Georgina KOPASZ, Szeged (HU); Ildikó FEKETE, Szeged (HU); Péter HORVÁTH, Szeged (HU); Farkas SÜKÖSD, Szeged (HU)

(21) Appl. No.: 18/853,413

(22) PCT Filed: Apr. 3, 2023
(86) PCT No.: PCT/HU2023/050014

§ 371 Date: Oct. 1, 2024

(30) Foreign Application Priority Data
Apr. 1, 2022 (HU) P2200102
May 17, 2022 (HU) P2200162

(51) Int. Cl. C12N 15/85; A01K 67/0275; C12N 15/113

(52) U.S. Cl. CPC C12N 15/8509; A01K 67/0275; C12N 15/113; A01K 2217/206; C12N 2015/859; C12N 2800/90; C12N 2820/007; C12N 2830/205

(57) ABSTRACT

The invention relates to an expression vector operable in vertebrate liver cells, having an expression cassette with a bidirectional promoter, driving operably linked protein expression by a first side and a second side, a first expression unit, under the control of the first side of the promoter, said first expression unit comprising a positive selectable marker gene, a second expression unit, under the control of the second side of the promoter, comprising an ORFeus reporter element wherein said ORFeus reporter element comprises a gene encoding L1-ORF land a retrotransposition reporter gene encoding a retrotransposition reporter protein, wherein preferably the retrotransposition reporter protein from said retrotransposition reporter gene is provided only when the ORFeus reporter element is subject to retrotransposition. The invention also relates to transgenic animals which are useful to detect somatic retrotransposition.

### FIELD OF THE INVENTION

The present invention relates to an innovative genomic technology to assess a new, hitherto unknown dimension of genotoxic effects of chemicals.

### BACKGROUND ART

Cancer incidence has been increasing in recent years in the European Union, due to the ageing population and other partly known factors, including emerging risks from chemicals in the environment. In the ageing population inherited genetic determinants may increase predominantly the incidence of familial forms of cancer. However, the impact of environmental factors can also be significant due to increased chemical use and pollution (Belpomme, Irigaray et al. 2007, Madia, Worth et al. 2019). In line with this, a statistical study of nearly 50 000 Scandinavian twins indicating that it is environmental factors rather than the inherited genetic determinants that make a larger contribution to susceptibility to most types of neoplasms (Lichtenstein, Holm et al. 2000). Known genotoxic chemicals cause DNA damage which in a somatic cell may result in mutations potentially leading to malignant transformation. However, the sporadic occurrence of cancer is often not explained by exposure to known genotoxic agents (such as occupational exposure to genotoxins, tobacco smoke, etc.).

A new research direction is based on the recognition that genotoxic effects may also be mediated through endogenous L1 (LINE1) retrotransposons. To our knowledge, L1 retrotransposons are the only currently active mobile genetic elements in the human genome. An active full-length L1 element is ˜6 kb in length, and encodes two open reading frames (ORFs). The structure and transposition of L1 elements are outlined in FIG. 1A and reviewed in reference (Beck, Garcia-Perez et al. 2011). In spite of the fact that the human and rodent L1 elements have evolved independently for approximately 80 million years, most human and mouse L1 sequences can be functionally exchanged (Wagstaff, Barnerssoi et al. 2011). L1 transpositional activity is able to alter the genomic structure in myriad ways. For example a new L1 insertion may directly influence the transcript levels of nearby genes by promoter addition or disruption/introduction of cis-regulatory elements (reviewed in (Feschotte 2008)). For a long time, L1 elements were thought to be active only in the germline and early embryo. Such germline activity accelerates the evolution of the mammalian genomes (Feschotte 2008). L1 activity is completely abolished early in embryonic development and remains undetectable in most normal somatic tissues due to the large number of partially revealed cellular mechanisms that protect against somatic L1 activity.

### L1 Defense Mechanisms in Somatic Cells

Without being exhaustive, we highlight some of the most important known L1 defense mechanisms operating in somatic cells (FIG. 1B). Upon the action of the Piwi-interacting RNA (piRNA) signaling pathway in germ cells, repressive heterochromatin is forming on the L1 sequences that is passed on to somatic cells (Aravin, Sachidanandam et al. 2008, Ozata, Gainetdinov et al. 2019, Mahadevan, Kumar et al. 2020, Yang, Lan et al. 2020). As long as this inherited heterochromatin is not released in somatic cells it continuously inhibits the expression of L1 elements. Directly in somatic cells, the SIRT6 (Van Meter, Kashyap et al. 2014) and SIRT7 (Vazquez, Thackray et al. 2019) proteins induce heterochromatinization and expressional silencing on L1 copies by different mechanisms. In somatic cells, several other L1 inhibitory processes also operate at the post-transcriptional level. Microprocessor, a protein complex that cleaves pri-microRNA structures during the normal maturation of microRNAs, is also able to recognize a pri-microRNA like hairpin structure in the L1 transcript and inhibits L1 expression by cleaving it (Heras, Macias et al. 2013). Similarly, the microRNA miR-128 containing RISC complex can bind to L1 transcripts in the cytoplasm and trigger their degradation (Hamdorf, Idica et al. 2015). Proteins involved in antiviral defense often have an L1 inhibitory effect as well. This includes the mov10 helicase that interacts with L1-ribonucleoprotein (L1-RNP) and together with other antiviral proteins forms cytoplasmic stress granules leading to subsequent L1-RNP degradation (Li, Zhang et al. 2013). Most of the proteins of the APOBEC3 deaminase family also have inhibitory effects on L1s. For example, APOBEC3A converts cytosine to uracil in the first strand of L1 cDNA, triggering its degradation (Richardson, Narvaiza et al. 2014). DNA repair factors also often affect L1 retrotransposition. For example, BRCA1, an E3 ubiquitin ligase that plays a key role in many DNA repair pathways, directly inhibits the frequency of L1 retrotransposition by protecting the replication forks it frequently targets (Mita, Sun et al. 2020). In addition, BRCA1 also suppresses ORF2 translation through association with its mRNA in the cytoplasm (Mita, Sun et al. 2020). Some cell cycle regulators are also involved in the somatic control of L1 elements. Among them, we can highlight the P53 protein, whose potent L1 activity inhibitory effect has only recently been described. Studies have shown that, p53 restricts growth of L1 expressing cells but not their retrotransposition potential (Ardeljan, Steranka et al. 2020) and L1 expression correlates with TP53 mutant status in human cancer (Rodriguez-Martin, Alvarez et al. 2020, McKerrow, Wang et al. 2022).

Any external environmental effect or chemical affecting the body can in principle disrupt some of these defensive mechanisms. In turn, the resulting increase in L1 activity can generate cancer “driver” mutations in the given tissue, thereby promoting tumor evolution. Presumably, such effects contribute to a large extent to the incidence of sporadic cancer cases worldwide. This may help explain the prominen role of environmental factors in susceptibility to sporadic cancer (Lichtenstein, Holm et al. 2000). Indeed, it has recently become clear that L1 elements can be reactivated at any site in the body under pathological conditions. A major discovery of recent years is that proteins expressed by the L1 retrotransposon can be detected in nearly half of all cancerous lesions and in an even larger proportion of high-grade tumors (Rodic, Sharma et al. 2014). The authors of this publication also proposed the use of the L1-ORF1 protein as a tumor marker. The intracellular presence of L1 proteins is a prerequisite for L1 retrotransposition. Consistent with this, several driver mutations were detected in different tumor types caused by new somatic L1 integration events (Miki, Nishisho et al. 1992, Shukla, Upton et al. 2013, Doucet-O'Hare, Rodic et al. 2015, Ewing, Gacita et al. 2015, Rodic, Steranka et al. 2015, Rodriguez-Martin, Alvarez et al. 2020).

### Difficulties of Detecting Somatic L1 Activity

Currently, the best L1 reporter systems are the ORFeus-type reporters (Han and Boeke 2004), which, when retrotransposed, produce a strong EGFP (or another marker) expression permanently in the given cell and its progeny. Another reliable L1 reporter system that is not of this “lineage tracing” nature is not momentary available.

This “lineage tracing” nature of the reporter makes it problematic to monitor somatic L1 activity in germline-transgenic mouse models because L1 elements are active in gametes and early embryos and in consequence L1 reporters are getting activated during the early stages of development. As a result, reporter transgenic mice will be EGFP positive throughout their body, making them unsuitable for tracking somatic L1 activity. The single published example of germline-transgenic L1 reporter mouse model applied for chemical risk assessment was created by the pronuclear microinjection of a 8.8kb ORFeus DNA fragment into fertilized eggs of mice (Okudaira, Goto et al. 2011). The authors surveyed several transgenic founders in order to find one that had low background of spontaneous ORFeus retrotransposition during embryogenesis. They needed to do this to avoid early embryonic retrotranspositions that would render their system useless. This constraint simultaneously weakens the sensitivity of their system, as they are limited to use transgenic founders expressing very low levels of the ORFeus reporter. Nonetheless, their experiments cannot rule out the possibility that any of the experimental animals studied also have embryonic ORFeus retrotransposition events in any tissue of interest. The authors applied a semi-quantitative PCR assay to assess the intensity of ORFeus reporter retrotransposition upon treatments with chemicals (Okudaira, Okamura et al. 2013). Principally it cannot be excluded that the measured values also include here the germline and early embryonic retrotransposition events and may not reflect well the chemical treatment induced somatic retrotranspositions.

Mammalian tissue culture could be an alternative to studies in mouse models. However, the feasibility of investigating cellular mechanisms that operate in primary somatic cells protecting against L1 retrotransposition is questionable in this system. Under healthy conditions, L1 activity in normal somatic cells is virtually zero. However, most of the laboratory cell lines are of tumor origin or have been cultured for a long time, and the L1 defense mechanisms are partially or completely inoperative in them. Freshly isolated primary cells, which may be an alternative, cannot usually be maintained in culture for a sufficient period of time.

Measurement of somatic L1 activity in germline-modified mouse models is problematic, as L1 elements are active in germ cells and early embryos, and thus all L1 reporters are activated early in development. Models created so far are inappropriate for the study of somatic retrotransposition activity.

### BRIEF DESCRIPTION OF THE INVENTION

The invention relates to an expression vector (preferably a plasmid) operable in vertebrate liver cells, preferably mammalian liver cells, preferably hepatocytes, said vector comprising an expression cassette flanked by a pair of genomic integration sequences, said cassette comprising
a mammalian, preferably human bidirectional promoter, driving operably linked protein expression by two sides of the promoter, a first side and a second side,
a first expression unit, under the control of the first side of the promoter, said first expression unit comprising
   a positive selectable marker gene allowing, once expressed in the liver cells, positive selection of the cells,
a second expression unit, under the control of the second side of the promoter, comprising an ORFeus reporter element wherein said ORFeus reporter element comprises
   a gene encoding LINE1-ORF1 (L1-ORF1 or ORF1 in short) and optionally a further gene encoding LINE1-ORF2 (L1-ORF2 or ORF2 in short), and
   a retrotransposition reporter gene encoding a retrotransposition reporter protein.

Said ORFeus reporter element is transcribed from the second side of the promoter once the expression cassette is stably integrated into the genome of the transgenic liver cell and said ORF protein(s) is/are expressed.

Preferably, a retrotransposition reporter upon retrotransposition is modified to report on the retrotransposition event.

Preferably, a retrotransposition reporter protein from said retrotransposition reporter gene is provided (i.e. expressed) only when the ORFeus reporter element is subject to retrotransposition in the genome of the transgenic liver cell.

Preferably, a retrotransposition reporter gene is restored in a different genomic site when the ORFeus reporter element is subject to retrotransposition in the genome of the transgenic liver cell.

Thus, when retrotransposition occurs, an intact retrotransposition reporter protein is expressed whereby the retrotransposition event is detectable.

Preferably, the ORFeus reporter element also comprises a termination signal between the retrotransposition reporter gene and the genomic integration sequence flanking the second expression unit.

In particular, the expression vector comprises a deficiency-complementing marker gene as a positive selectable marker and is useful for in vivo somatic transgenesis of the liver of a vertebrate animal, preferably mammal, particularly preferably murine including mice, said animal being deficient in the trait provided by the marker gene. In particular, the deficiency-complementing marker gene is the Fah gene.

In an embodiment the ORFeus reporter element comprises, in reverse orientation, an expression unit for the retrotransposition reporter gene,
said expression unit for the retrotransposition reporter gene comprising a retrotransposition reporter blocking sequence which is removed in the retrotransposition process wherein a retrotransposition reporter is provided only when a retrotransposition occurs. In a preferred embodiment the retrotransposition reporter blocking sequence is an intron within the retrotransposition reporter gene.

In an embodiment the ORFeus reporter element comprises in sense (forward) orientation an ORF protein expression unit comprising the gene encoding one or two ORF protein(s) and the 3′UTR.

In an embodiment the ORFeus reporter element comprises, from the second side of the promoter, a LINE1 ORF1 coding sequence (L1-ORF1) and optionally a LINE1 ORF2 coding sequence (L1-ORF2), a 3′ untranslated region (3′UTR), and, in reverse orientation, an expression unit for the retrotransposition reporter gene.

In a preferred embodiment the retrotransposition reporter blocking sequence is an intron (retrotransposition reporter blocking intron) which is in normal (forward or sense) orientation in relation to the second side of the bidirectional promoter. In a preferred embodiment the expression unit for the retrotransposition reporter gene comprises a retrotransposition reporter promoter and, under the control thereof, a retrotransposition reporter gene and a termination signal, preferably a polyA sequence in antisense (reverse) orientation (reverse orientation termination sequence or reverse polyA), said retrotransposition reporter gene comprising an intron in sense (forward) orientation which is removed during transcription of the element from the second side of the bidirectional promoter. In a highly preferred particular embodiment the retrotransposition reporter blocking intron is a human gamma globin intron 2 or a variant thereof.

A retrotransposition reporter gene encodes the retrotransposition reporter protein.

In a preferred embodiment, the ORFeus reporter element comprises, in reverse orientation, an expression unit for the retrotransposition reporter gene,
said expression unit for the retrotransposition reporter gene comprising a first exon, a second exon and between them an intron which is removed in the retrotransposition process wherein a retrotransposition reporter protein is provided only when a retrotransposition occurs.

In a preferred embodiment, the expression unit for the retrotransposition reporter gene in reverse orientation comprises
a first exon of a visible marker gene, preferably a fluorescent marker gene and,
a second exon of the visible marker gene, preferably the fluorescent marker gene
wherein upon retrotransposition, once linked with the polypeptide encoded by the second exon, a visible retrotransposition reporter, preferably a fluorescent protein, is expressed from the visible marker gene.

In a preferred embodiment, the second exon of the visible marker gene has, operably linked thereto, a coding region for a peptide tag which serves as an epitope for an antibody specific for the particular peptide tag.

In a preferred embodiment, the intron in the expression unit for the retrotransposition reporter gene is relocated to increase the length of the second exon and decrease the length of the first exon thereby providing an epitope within the second exon which serves as an epitope for an antibody specific for the second exon.

In an embodiment the ORFeus reporter element comprises an ORF1 coding sequence and a 3′UTR in forward (sense) orientation, an expression unit for the retrotransposition reporter gene in reverse (antisense) orientation with the retrotransposition reporter blocking intron in sense (forward) orientation, and the termination sequence at the 3′ end of the second expression unit. The termination sequence at the 3′ end of the second expression unit comprises or is a polyA signal.

In a further embodiment the ORFeus reporter element, in particular the ORF protein expression unit comprises an ORF1 and an ORF2 coding sequence and a 3′UTR (autonomous system). In a further embodiment the ORFeus reporter element comprises TF monomers, e.g. comprises TF monomers and an ORF1 coding sequence or TF monomers and an ORF1 and an ORF2 coding sequence.

While ORF1 is essential, ORF2 can be omitted in certain embodiments (non-autonomous system). The ORFeus reporter variants that do not express the ORF2 protein have an advantage over the full-length reporter in that they do not function autonomously. In this case, the ORF2 protein, which is also required for retrotransposition, is expressed from endogenous L1 copies. Thus, the non-autonomous system may be used to report on the expression status of endogenous L1 copies.

In a highly preferred embodiment the ORFeus reporter variant used is derived from a mouse retrotransposon, in a particular embodiment from the pWA125 construct. In a preferred embodiment L1-ORF2 is deleted from the pWA125 construct.

In a further preferred embodiment, the reporter cassette has been inserted in its 3UTR region.

The TF monomer region, if present, functions as a promoter. Wherein the TF monomer region is not present, ORFeus expression will be driven solely by the bidirectional promoter (preferably second side), preferably from the HADHA/B promoter.

In further embodiments the ORFeus reporter element is derived from a mammalian L1 element, preferably a rodent, e.g. murine or a monkey or ape, e.g. human L1 element. In a preferred embodiment the ORFeus reporter element is sequence-optimized e.g. to increase retrotransposition frequency, avoid suppression process by the cell etc.

In an embodiment the termination signal at the end of the ORFeus reporter element preceding the genomic integration sequence flanking the second expression unit is a polyA polyadenylation signal, preferably an SV40 derived or SV40 polyA signal. In particular, the polyA signal is a nucleotide sequence having at least 70%, preferably at least 80%, more preferably at least 90% sequence identity with nucleotides 6141 to 6382 of SEQ ID NO: 9.

In a particular embodiment the ORF1 (protein) coding sequence is a nucleotide sequence which is at least 60%, preferably at least 70%, more preferably at least 80%, in particular at least 90% identical with SEQ ID NO:20, or with nucleotides 2056 to 3171 of SEQ ID NO: 8 or SEQ ID NO: 9 or SEQ ID NO: 11 or with nucleotides 280-1395 of SEQ ID NO: 10, wherein the protein encoded has an ORF1 function. In a particular embodiment the ORF1 coding sequence is a nucleotide sequence which encodes a protein sequence which is at least 60%, preferably at least 70%, more preferably at least 80%, in particular at least 90% identical with a protein sequence encoded by SEQ ID NO:21.

In a particular embodiment the ORF1 protein has an amino acid sequence of SEQ ID NO:21 or an amino acid sequence which is at least 70%, more preferably at least 80%, in particular at least 90% identical therewith, wherein the protein encoded has an ORF1 function.

In a particular embodiment the ORF2 (protein) coding sequence is a nucleotide sequence which is at least 60%, preferably at least 70%, more preferably at least 80%, in particular at least 90% identical with SEQ ID NO:22 (or with nucleotides 3212-7057 of SEQ ID NO:8); or in a particular embodiment the ORF2 coding sequence is a nucleotide sequence which encodes a protein sequence which is at least 60%, preferably at least 70%, more preferably at least 80%, in particular at least 90% identical with a protein sequence encoded by SEQ ID NO:23.

In a particular embodiment the ORF2 protein has an amino acid sequence of SEQ ID NO:23 or an amino acid sequence which is at least 70%, more preferably at least 80%, in particular at least 90% identical therewith, wherein the protein encoded has an ORF2 function.

In a particular embodiment the 3′ UTR is a nucleotide sequence which is at least 60%, preferably at least 70%, more preferably at least 80%, in particular at least 90% identical with nucleotides 3189 to 3611 of SEQ ID NO: 9.

In a particular embodiment the TF monomer, once present, is a nucleotide sequence which is at least 60%, preferably at least 70%, more preferably at least 80%, in particular at least 90% identical with nucleotides 235 to 1834 of SEQ ID NO: 9. The TF monomer region is shown separately in SEQ ID NO:24 (same as in SEQ ID Nos: 8-9 and 11).

Variants for ORFeus reporter elements are known in the art (References for mouse: O'Donnell, Kathryn A. et al. (2013). Controlled insertional mutagenesis using a LINE-1 (ORFeus) gene-trap mouse model. PNAS, 110(29): E2706-E2713, https://www.pnas.org/doi/full/10.1073/pnas.1302504110; and for human: An, Wenfeng et al. (2011). Characterization of a synthetic human LINE-1 retrotransposon ORFeus-Hs. Mobile DNA, 2(1): 2. https://www.ncbi.nlm.nih.gov/pmc/articles/PMC3045867/)

In an embodiment the expression unit for the retrotransposition reporter gene in reverse (antisense) orientation comprises a promoter in particular a mammalian promoter for protein expression in mammals, e.g. a cytomegalovirus immediate-early promoter or CMV promoter, in particular a promoter having a nucleotide sequence which is at least 70%, preferably at least 80%, more preferably at least 90% identical with nucleotides 5526 to 6110 of SEQ ID NO: 9 (antisense or reverse orientation).

In an embodiment the expression unit for the retrotransposition reporter gene in reverse (antisense) orientation comprises a first exon of a visible marker gene, preferably a fluorescent marker gene e.g. EGFP, in particular wherein the sequence of said first exon having a nucleotide sequence which is at least 60%, preferably is at least 70%, more preferably at least 80%, particularly preferably at least 90% identical with nucleotides 4943 to 5476 of SEQ ID NO: 9 (antisense or reverse orientation) OR nucleotides 635 to 1168 of SEQ ID NO: 12 (sense or forward orientation).

The exemplary amino acid sequence encoded by the first exon is given by SEQ ID NO:13. Thus the retrotransposition reporter encoded by the first exon is, in particular, a polypeptide having an amino acid sequence given by SEQ ID NO:13 or a sequence being at least 70%, more preferably at least 80%, particularly preferably at least 90% identical therewith and, once linked with the polypeptide encoded by the second exon, forming a fluorescent protein, preferably having EGFP function of green fluorescence.

In an embodiment the expression unit for the retrotransposition reporter gene in reverse (antisense) orientation comprises an intron between the reverse orientation first exon and second exon of the visible marker gene, wherein the intron is in forward (sense) orientation and the sequence of which has a nucleotide sequence which is at least 60%, preferably is at least 70%, more preferably at least 80%, particularly preferably at least 90% identical with nucleotides 7917 to 8818 of SEQ ID NO: 8 or 4041 to 4942 of SEQ ID NO: 9. In SEQ ID NO: 12 this intron is shown in the reverse orientation (nucleotides 1169 to 2070). In a preferred embodiment the intron is or is derived from the hGamma Globin intron 2.

The orientation of the intron is opposite to the exons of the retrotransposition reporter gene e.g. EGFP, so that when the mRNA is transcribed from the antisense strand driven by the second side of the bidirectional promoter, e.g. the HADHB side of the HADHA/B promoter, the mRNA from the antisense strand of the reporter gene is spliced and the intron is removed whereas no reporter protein can be transcribed from this mRNA. Only when a reverse transcription and transposition event occurs due to the concerted effect of ORF1 and ORF2 proteins, the coding sequence of the retrotransposition reporter gene together with its own promoter is integrated into a site, different from the original one, of the liver cell genome, in the form of a DNA and the coding strand is restored.

In an embodiment the expression unit for the retrotransposition reporter gene in reverse (antisense) orientation comprises a second exon of a visible marker gene, preferably a fluorescent marker gene e.g. EGFP, in particular wherein the sequence of said second exon having a nucleotide sequence which is at least 60%, preferably is at least 70%, more preferably at least 80%, particularly preferably at least 90% identical with nucleotides 3855 to 4040 of SEQ ID NO: 9 (antisense or reverse orientation) OR nucleotides 2071 to 2256 of SEQ ID 12 (sense or forward orientation).

The exemplary amino acid sequence encoded by the second exon is given by SEQ ID NO: 14. Thus the retrotransposition reporter encoded by the second exon is, in particular, a polypeptide having an amino acid sequence given by SEQ ID NO:14 or a sequence being at least 70%, more preferably at least 80%, particularly preferably at least 90% identical therewith and, once linked with the polypeptide encoded by the first exon, forming a fluorescent protein, preferably having EGFP function of green fluorescence.

In an embodiment the expression unit for the retrotransposition reporter gene in reverse (antisense) orientation also comprises a polyA signal in reverse orientation in view of the HADHB promoter side as this sequence serves as a polyA signal for the retrotransposition reporter gene expression unit. In a preferred embodiment this is a hsvTK polyA polyadenylation signal. In a particular embodiment the polyA sequence of which has a nucleotide sequence which is at least 70%, more preferably at least 80%, particularly preferably at least 90% identical with nucleotides 2260 to 2483 of SEQ ID NO: 12 (forward sequence or sense strand) or 7504 to 7727 of SEQ ID NO: 8 (reverse sequence or antisense strand), having the polyA function.

An example for the expression unit for the retrotransposition reporter gene, in particular an EGFP expressing unit with the human gamma globin intron 2 is given by SEQ ID NO: 12. In this example the elements of the expressing unit are as follows:
nucleotides 1 to 585: CMV promoter,
nucleotides 635 to 1168: exon 1 of EGFP,
nucleotides 1169 to 2070: human gamma globin intron 2,
nucleotides 2071 to 2256: exon 2 of EGFP,
nucleotides 2260 to 2483: hsvTK polyA polyadenylation signal.

In the present invention an expression unit has the sequence of at least 70%, preferably at least 80%, particularly preferably at least 90% identical with nucleotides 1 to 2483 of SEQ ID NO: 12, provided that the function of the above elements and in case of the protein encoded by exon 1 and exon 2 the fluorescence is maintained.

Preferably in the invention the positive selectable marker gene and the ORF reporter located on the same expression construct are expressed with balanced expression.

The skilled person will understand that further regulatory sequences may form part of the ORFeus reporter element and may add further features to the expression cassette and the expression vector of the invention.

In a preferred embodiment the vertebrate is a mammalian experimental animal, preferably the animal is a rodent, preferably murine. Thus, the promoter is operable in the experimental animal. In particular, the expression vector is operable in liver cells of an animal in vivo.

In a further aspect the invention relates to said animal comprising the expression construct stably integrated in its genome.

In an embodiment the positive selectable marker gene is a deficiency-complementing marker gene which provides a function in which the cells, in particular the liver cells of the animal are deficient (e.g. said gene is not functional in the cells of the animal, i.e. said cells are deficient in said gene), which impairs a population of cells of an organ unless a condition, e.g. presence of a compound is provided. Selection is carried out by providing a condition which is unfavorable to the deficient cells e.g. by withdrawal of said compound from the environment of the cells. Under such conditions the cells expressing such deficiency-complementing selectable marker gene have growth advantage over the deficient cells.

Very preferably the positive selectable marker gene is the Fah selection marker which provides growth advantage to cells over cell lacking said marker (e.g. Fah^{-/-} cells) in the absence of a 4-Hydroxyphenylpyruvate dioxygenase (HPPD) inhibitor e.g. nitisinone (NTBC). In a particularly preferred embodiment the Fah selection marker gene has the nucleotide sequence of SEQ ID NO: 15 or a selection marker gene having the sequence which has at least 70%, preferably 80%, more preferably 90%, particularly preferably at least 95% sequence identity therewith.

In a preferred embodiment the Fah selection marker gene has a sequence encoding the Fah protein, in particular an amino acid sequence which has at least 70%, preferably 80%, more preferably 90%, particularly preferably at least 95% sequence identity with SEQ ID NO: 16.

In particular the positive selectable marker gene is the Fah gene and the experimental animal is a murine the liver of which is subjected to somatic genome editing.

In a preferred embodiment the vector comprises transposon ITRs for genomic integration and is used together with a transposase to obtain transgenic liver cells having the expression cassette stably integrated in their genome whereas the expression of the marker gene provides selective advantage of the transgenic cells in the liver to overgrow deficient cells. Via driving bidirectional and concerted expression of the elements in the transgenic liver of the animal, using an EF1 intron for harboring silencer sequences, this versatile system is particularly useful for expressing a gene of interest with the simultaneous effective silencing, particularly via artificial microRNA, of a gene in the genome of the animal.

Preferably, the bidirectional promoter is a promoter which provides physiological expression level, e.g. the expression level provided by said promoter is similar, i.e. is at most about 2 orders of magnitude higher than the expression of, preferably at most about 1 order of magnitude higher than the expression of a housekeeping gene and at most about 1 order of magnitude lower than the expression of the housekeeping gene. In a particular embodiment, the housekeeping gene, to which the expression levels are compared, is the ribosomal protein L27 (Rpl27).

In an embodiment the expression level provided by the bidirectional promoter is more than 0.05 times, preferably 0.1 times and less than 10² times, preferably less than 50 times, preferably 10 times (particularly preferably 0.1-10 times) of that of the housekeeping gene, preferably coding L27 protein sequence.

Preferably, the bidirectional promoter is a mammalian HADHA/B promoter, preferably a human HADHA/B promoter.

In a particular embodiment the expression level provided by the HADHA/B promoter is in the physiological range of expression, i.e. is in comparison with the expression level of a housekeeping gene, in particular the Rpl27 housekeeping gene or a housekeeping gene expressed in the same order of magnitude as Rpl27, the expression level provided by the HADHA/B promoter (i.e. the expression level of the genes driven by the HADHA/B promoter) is similar, i.e. is at most 2 orders of magnitude higher than the expression of, preferably at most 1 order of magnitude higher than the expression of the Rpl27 housekeeping gene. In a particular embodiment, the housekeeping gene, to which the expression levels are compared, is the ribosomal protein L27 (Rpl27).

Preferably the expression level provided by the HADHA/B promoter is more than 0.5 times and less than 10² times, e.g. 1 to 100 times, preferably less than 50 times, preferably 1 to 10 times of that of a housekeeping gene, e.g. L27.

In particular, the expression levels provided by the bidirectional promoter should be no less than 1 order of magnitude lower than the normal physiological value of the expression of the L27, and no more than 1 or 2 order of magnitude higher than the normal physiological value of the expression of the L27.

In a particularly preferred embodiment the HADHA/B promoter has a sequence identity of at least 70% or 80% or 85% or 90% or 95% with SEQ ID NO: 17 (HADHA/B).

For sake of description and illustration the HADHA/B nucleotide sequence is split up into two parts in e.g. SEQ ID NOs 6 and 8 and is shown by nucleotides 1 to 180 of SEQ ID NO. 6 and 7 (indicated as coding strand for HADHA side) and nucleotides 1 to 210 of SEQ ID NO. 8 to 11 (indicated as coding strand of HADHB side). As to preferred embodiments the same homology, i.e. the same identity ranges apply for both parts as given above for SEQ ID NO. 17. It will be understood, however, that this, somewhat artificial division into two parts serves illustrative purposes and what is important for the operation of the expression cassette is the presence of the HADHA/B promoter itself operably linked to both expression units. As will be readily understood by a person skilled in the art the expression starts at the start codon and its first nucleotide, i.e. the start site is given in the sequence listing as nucleotide 156 for the HADHA side (SEQ ID NOs: 6 to 7) and nucleotide 196 for the HADHB side (SEQ ID NOs: 8 to 11).

The first side of the bidirectional promoter directs the expression of the positive selectable marker gene. In a preferred embodiment the positive selectable marker gene is a deficiency-complementing marker gene e.g. as defined above. The deficiency-complementing marker gene becomes functional in the cells in which the expression construct is stably integrated. Typically the gene expressing a protein with the same function is not functional in other cells against which selection is carried out (said cells are deficient in said gene), unless a particular condition is provided. Changing the condition provides selective advantage to the cells having functional deficiency-complementing marker gene.

In a particularly preferred embodiment the positive selectable marker gene is a Fah gene, e.g. a Fah gene as defined herein.

In a preferred embodiment the expression construct comprises an integration detecting marker gene.

Preferably the integration detecting marker gene is present in the first expression unit of the expression cassette, preferably between the positive selectable marker gene and the A side of the HADHA/B promoter.

In a particularly preferred embodiment the integration detecting marker gene is a visible marker gene, preferably a fluorescent marker gene (different from the retrotransposition reporter gene). In a highly preferred embodiment the marker gene is the mCherry fluorescent marker gene.

In a particular embodiment the mCherry marker gene has a sequence identity of at least 70% or 80% or 85% or 90% or 95% with nucleotides 184-561 of SEQ ID NO. 6 or 7 (first exon) and nucleotides 1418-1747 of SEQ ID NO. 6 or 1751 to 2080 of SEQ ID NO. 7 (second exon) wherein the mCherry fluorescent marker gene is functional once expressed.

Preferably the intron is located within the integration detecting marker gene or within the positive selection marker gene.

Optionally the vector also comprises an intron comprising integration site to insert one or more silencer sequence(s), optionally said silencer sequence being inserted into said integration site.

In a preferred embodiment the intron (EF1-intron) is at least 300 nucleotide long and has 5′ and 3′ splice sites and a branch site of the first intron of a human eukaryotic translation elongation factor 1 alpha 1 (EEF1A1), and has at least 60% identity with the corresponding sequence thereof, to ensure intronic expression of the one or more silencer sequence(s) (EF1 intron).

In a preferred embodiment the intron (EF1-intron) is at least 400, preferably 500, more preferably 600 nucleotide long and has 5′ and 3′ splice sites and a branch site of the intron and has at least 70%, preferably at least 80%, identity with the corresponding sequence part of the human eukaryotic translation elongation factor 1 alpha 1 (EEF1A1), in particular with SEQ ID NO. 18 or nucleotides 562-1417 of SEQ ID NO. 6 to ensure intronic expression of the one or more silencer sequence(s) (an EF1 intron). Particular embodiments are as defined above.

In a preferred embodiment gene silencing is artificial microRNA-based (amiR-based) gene silencing.

Particularly preferably the silencer sequence is artificial microRNA providing sequences (amiR elements). Preferably, these regulatory sequences (artificial microRNAs) can silence any arbitrary target gene in the host cell genome. Artificial microRNA providing sequences (amiR elements) comprise a microRNA coding sequence, which, upon expression and maturation, result in active matured microRNA (miRNA).

Once the silencer sequence, preferably the amiR sequence is present in an intron its maturation does not interfere with the expression of either the selection marker or the gene of interest.

In an embodiment the silencer sequence is capable of silencing
a wild-type of the gene of interest, or
a gene interfering with the effect of the gene of interest,
a gene inhibiting, e.g. reducing the activity of the gene of interest.

In a particular embodiment the silencer sequence is selected from an amiR sequence comprising a microRNA 5′ and 3′ flanking sequence and a stem-loop-guide sequence of a microRNA specific to a sequence to be silenced.

In a particular embodiment the silencer sequence is an amiR sequence to which the flanking regions are given by SEQ ID NO: 19 whereas the specific microRNA parts may vary.

Preferably gene silencing is adjusted to gene silencing in vivo in the animal, preferably mammalian, in particular murine, e.g. mouse liver.

Preferably one or more amiR elements is/are present in the first expression unit, within the EF1 intron, controlled by the HADHA side, i.e., on the same side as the positive selectable marker gene; for example, it is present in the integration detecting marker gene, between the positive marker gene and the bidirectional promoter.

Preferably one or more amiR elements is/are present in the first expression unit, within the EF1 intron, controlled by the HADHA side, i.e., on the same side as the positive selectable marker gene; for example, it is present between the positive marker gene and the bidirectional promoter.

In a particularly preferred embodiment, the expression vector according to the invention does not comprise an endogenous gene-silencing element, in particular an amiR element.

Preferably the expression is tag-free expression. The use of HADHA/B gives the possibility for the marker-linked expression of an untagged native protein or a mutant protein isoform, for example untagged expression of the ORF and optionally the ORF2 proteins.

The role of the transposon system used herein is to provide transfer of the expression construct of the invention into the genome of the liver cells used in the present invention to create a transgenic liver in an animal. In this methodology in case of class II transposons (DNA transposons), by providing an appropriate transposase, the expression construct between the ITRs of the transposon system used is integrated stably into the genome in a controllable manner.

Thus, in a preferred embodiment in the expression vector the flanking genomic integration sequences are a pair of transposon inverted terminal repeats (ITRs).

Preferably the transposon the ITR of which is used is a hyperactive transposon system (of high gene insertion activity). Preferably the transposon system is piggyBac (PB) or Sleeping Beauty (SB) transposon system. The skilled person will be aware that further transposon systems may be made appropriate (like for example the Tol2 transposon system) particularly if they have a sufficiently high gene insertion activity, e.g. made “hyperactive”.

In a particular embodiment the 5′ inverted terminal repeat is a PB 5′ ITR, preferably an ITR having the sequence identity of at least 70% or 80% or 85% or 90% or 95% with nucleotides 3307-3612 of SEQ ID NO. 6 (coding strand of HADHA side).

In a particular embodiment the 3′ inverted terminal repeat is a PB 3′ ITR, preferably an ITR having the sequence identity of at least 70% or 80% or 85% or 90% or 95% with nucleotides 10430 to 10666 of SEQ ID NO. 8 (coding strand of HADHB side).

The skilled person has a general knowledge about known necessary elements of a transcription unit.

Preferably the genes are followed by a polyadenylation signal (i.e. transcription unit end) in each transcription unit. In a preferred option the bGH polyadenylation signal is used (see e.g. nucleotides 3071 to 3298 of SEQ ID NO: 6 or nucleotides 3404 to 3631 of SEQ ID NO: 7).

In a particularly preferred embodiment an expression unit, preferably in the first expression unit and driven by the HADHA side, comprises a visible marker gene as an integration detecting marker gene, also for detecting integration of the expression from the HADHA side, and the visible marker gene comprises the EF1 intron comprising the silencer sequences, preferably the amiR silencer sequences.

Preferably the visible marker gene is a fluorescent marker gene. In particular the fluorescent marker gene is the mCherry fluorescent marker gene.

In a highly preferred embodiment, the mCherry coding sequence (CDS) is operably linked to the mouse fumaryl-aceto-acetate dehydrogenase (Fah) CDS to provide bicistronic expression. Preferably operable linking is carried out by a peptide tag, preferably a T2A peptide tag.

In a further aspect the invention also relates to a kit of vectors comprising any one of the expression vectors defined above and a helper vector comprising an expression unit which, when expressed in the same cell in which the expression vector is present, promotes integration of the expression unit into the genome of the cell.

Preferably,
the expression vector is an expression vector of any of the vectors defined above, said vector having transposon inverted terminal repeats (ITRs) as genomic integration sequences and
the helper vector encodes a helper enzyme effecting genomic integration of the expression unit by acting on the terminal repeats, preferably the helper enzyme being a transposase.

Preferably the terminal repeats are transposon ITRs (preferably piggyBac (PB) or Sleeping Beauty (SB) transposon ITRs) and the helper enzyme is transposase (preferably PB or SB-transposase, respectively).

The invention also relates to a method for preparing transgenic cells comprising administering the expression vector of the invention and a helper vector comprising an expression construct which, when expressed in the same cell in which the expression vector is present, promotes integration of the expression unit into the genome of the cell.

Preferably the helper vector is as defined herein or in any paragraph above.

In a preferred embodiment the vectors are co-administered to the animal as defined herein by a hydrodynamic injection which has been found particularly preferred. Methods for hydrodynamic injection are known for a person skilled in the art. An example is tail vein hydrodynamic injection, e.g. as described herein.

In another embodiment the vectors are encapsulated and co-administered intravenously. The vectors (preferably plasmids) can be encapsulated, for example, into lipid nanoparticles or virus capsids.

Other administration routes are within the skills of a person skilled in the art.

The invention also relates to a method for preparing a transgenic animal having a liver populated with transgenic liver cells (preferably hepatocytes) wherein said transgenic liver cells (preferably hepatocytes) overexpress the gene of interest,
comprising the steps of
   providing a vertebrate, preferably a mammalian animal in which the selectable marker gene is deficient (dysfunctional), wherein in lack of such selectable marker gene function the liver cells of the animal are impaired,
   providing a population of transgenic liver cells in the animal by co-administering the expression vector comprising a deficiency-complementing selectable marker gene and a helper vector as defined herein thereby obtaining said population comprising the expression unit of the expression vector functionally integrated into their chromosomes, wherein both the selectable marker gene and the gene of interest is expressed driven by the bidirectional promoter,
   providing selective advantage to the transgenic cells having the deficiency-complementing selectable marker gene integrated into their genome,
   allowing the transgenic liver cells to proliferate in the liver, whereas the amount of impaired liver cells is decreasing until transgenic liver is obtained in the mammalian animal.

Preferably the transgenic liver cells are prepared by administering the expression vector of the invention and a helper vector (preferably as defined herein) comprising an expression construct which, when expressed in the same cell in which the expression vector is present, promotes integration of the expression unit into the genome of the cell. In a preferred embodiment the vectors are co-administered by a hydrodynamic injection into the animals which has been found particularly preferred.

Preferably the expression vector comprises the integration detecting marker gene as defined herein.

In a further preferred embodiment the one or more silencing sequence(s) down-regulate a gene of the liver cells, preferably the one or more silencing sequence(s) is/are gene-specific silencer RNA(s), more preferably miRNAs.

In the transgenic animal the expression construct is a construct as defined herein.

The invention also relates to a use of the expression vector according to the invention for the preparation of a transgenic non-human vertebrate animal as defined herein.

In a preferred embodiment the one or more silencing sequence(s) down-regulate expression of a gene which would result in suppression of L1 retrotransposition as explained above. In an example p53 is silenced (see FIG. 1B) which has been suggested to have a role in protection against L1 activity.

Without limitation the following elements may be target of gene silencing:
Piwi-interacting RNA (piRNA) signaling elements,
SIRT6 and SIRT7,
Microprocessor (cleaving pri-microRNA structures),
RISC complex,
proteins of the APOBEC3 deaminase family,
BRCA1 etc.

In a particularly preferred embodiment, the expression vector according to the invention does not comprise an endogenous gene-silencing element, in particular an amiR element.

In a further aspect the invention relates to a transgenic liver cell comprising an expression cassette as defined herein, stably and operably integrated in its genome.

In a further aspect the invention relates to a transgenic non-human vertebrate, preferably mammalian experimental animal having a transgenic liver comprising somatic liver cells having a construct stably integrated into the genome, said construct comprising the expression cassette as defined herein. Preferably stable integration is carried out by a transposon system as defined herein.

Preferably said transgenic non-human vertebrate, preferably mammalian animal has a liver comprising cells having said construct stably integrated into the genome.

The invention also relates to a preparation prepared from the liver of the transgenic non-human vertebrate animal of the invention. Such preparation can be e.g. a tissue preparation prepared by obtaining a tissue part of the transgenic liver or a membrane preparation obtained by membrane preparation techniques.

The test animal in the present invention is non-human.

Preferably the test animal is a laboratory or experimental animal.

Preferably the test animal is a rodent, preferably murine.

Any testing is made under due ethical considerations of animal welfare.

The invention also relates to a use of the non-human vertebrate animal according to the invention for assessing alteration or modulation L1 retrotransposition activity in a vertebrate liver present in said test animal in which the expression vector is operable.

The invention also relates to a use of the non-human vertebrate animal according to the invention for measuring the level of modulation L1 retrotransposition activity in a vertebrate liver present in said test animal in which the expression vector is operable.

Modulation may result in increasing or decreasing the level of L1 retrotransposition activity.

The invention also relates to a use of the non-human vertebrate animal according to the invention for testing the effect of a test compound to modulate L1 retrotransposition activity in a vertebrate liver present in said test animal in which the expression vector is operable.

The invention also relates to a use of the non-human vertebrate animal according to the invention for testing the effect of a test compound to induce L1 retrotransposition activity in a vertebrate liver present in said test animal in which the expression vector is operable. Inducers (or activators) increase retrotransposition activity. Such compounds may also contribute to the formation of neoplastic cells, e.g. tumors, cancers etc.

The invention also relates to a use of the non-human vertebrate animal according to the invention for testing the effect of a test compound to reduce L1 retrotransposition activity in a vertebrate liver present in said test animal in which the expression vector is operable. In this case, if a higher baseline activity can be arrived at, e.g. by silencing a retrotransposition inhibitor sequence (retrotransposition inhibitor reduce retrotransposition activity) or by application of a full length (autonomous) ORFeus element, the inhibitors of retrotransposition activity can be measured.

The invention also relates to a method for testing a compound (e.g. screening a test compound) for its activity to modulate (e.g. induce or reduce) L1 retrotransposition activity in a transgenic animal having a transgenic liver comprising the expression construct of the invention as defined herein, in particular as defined above,
said method comprising
administering said test compound to said transgenic animal of the invention,
measuring retrotransposition activity by the level of retrotransposition in the transgenic cells of the liver in the animal.

In an embodiment the invention relates to a method for screening a test compound for activity to induce L1 retrotransposition activity in a transgenic animal having a transgenic liver comprising the expression construct of the invention as defined herein, in particular as defined above,
said method comprising
administering said test compound to said transgenic animal of the invention,
measuring the level of the retrotransposition in the liver of the transgenic animal.

Measuring the level of retrotransposition is carried out via the retrotransposition reporter gene, e.g. by the relocation of the retrotransposition reporter gene by retrotransposition or by expression of the retrotransposition reporter protein from the gene relocated by retrotransposition, in particular in the form of a full (complete) protein.

In a preferred embodiment the method comprises measuring the level of retrotransposition via
the amount (level, e.g. number) of cells expressing the retrotransposition reporter protein as a visible marker,
the amount (level, e.g. number) of cells expressing the retrotransposition reporter protein, e.g. by immunological detection.

The number of cells can be measured e.g. by cell counting or cell sorting or by tissue staining.

In a preferred embodiment the method comprises measuring the level of retrotransposition via measuring the amount (level) of retrotransposed reporter gene in the form of DNA, from which the intron has been removed, e.g. by qPCR.

Preferably the method comprises
measuring the level of the retrotransposition
   in the presence of the test compound, and
   in the absence of the test compound,
comparing the levels measured
   in the presence of the test compound, and
   in the absence of the test compound
assessing the compound as a modulator of L1 retrotransposition activity if the level of expression of the retrotransposition reporter protein is modified;
for example, as an inducer of L1 retrotransposition activity if the level of expression of the retrotransposition reporter protein is increased;
a reducer of L1 retrotransposition activity if the level of expression of the retrotransposition reporter protein is reduced.

### Definitions

The “ORFeus reporter element” is a modified endogenous L1 element that, when retrotransposed, generates a stable marker signal (e.g. EGFP or another marker) in the cell and its progeny. The ORFeus reporter functionality requires at a minimum that it expresses the L1-ORF1 protein and contains a retrotransposition reporter cassette integrated into its 3′UTR region in reverse orientation.

In further embodiments the ORFeus reporter element may comprise TF monomers which may serve as promoter, and may comprise L1-ORF2 protein coding sequence (autonomous ORFeus reporter element).

Furthermore, the retrotransposition reporter cassette may comprise an intron in the retrotransposition reporter gene which is spliced out during retrotransposition resulting in an intact or complete retrotransposition reporter gene and/or gene product.

A “construct” as used herein is an artificial (human-made) nucleic acid molecule comprising one or more expressible sequence(s) or cloning site(s) for insertion of said sequence(s) and one or more regulatory sequence(s) regulating said expression of at least one of said one or more expressible sequence(s).

An “expression vector”, preferably a DNA vector, as used herein is a construct which is able to replicate in a cell, preferably in a mammalian cell (or host), and having at least one origin of replication, a selectable marker, and a cloning site suitable for the insertion of a gene, as well as a promoter driving expression of said gene including translation into mRNA, preferably in said mammalian cell, and other necessary sequences like translation initiation sequence such as a ribosomal binding site, start codon, and termination sequences. Preferably the cell is a mammalian cell.

An “expression cassette” is used herein as a distinct part (or component) of the vector DNA useful for expression of one or more, preferably multiple genes in an operably linked or concerted manner, preferably from a single promoter, wherein the expression cassette directs the cell's machinery to make RNA and protein. The expression cassette is part of the expression vector and thus comprises every essential means (consisting of sequences) for the expression of the genes expressible from that expression cassette.

An “transcription unit” or “expression unit” as used herein is an expression cassette or a part thereof consisting of a gene and regulatory sequence driving expression of said gene in and by a transfected cell. In each successful transfection, the transcription unit or expression unit directs the cell's machinery to express said gene to make RNA (transcription) and preferably protein(s) (translation from RNA).

The transcription or expression unit is composed of one or more genes and at least one sequence controlling their expression, as well as one or more untranslated region. In a particular embodiment the unit comprises three components: a promoter sequence, an open reading frame, and ends in a 3′ untranslated region that, in eukaryotes, typically contains a polyadenylation site.

In the present invention in particular the expression unit is suitable for integration into the chromosome of a mammal in a functional (i.e. operable) manner i.e. that can provide its expression function when present in the chromosome.

“Transfection” as used herein is any method of gene transfer in which the genetic material is deliberately introduced into vertebrate, preferably mammalian cells. A particular method according to the invention is hydrodynamic injection.

An “integration site” in a nucleic acid, preferably in an expression vector, is a site comprising a sequence suitable for inserting another nucleic acid (insert), including opening (cutting) the nucleic acid at the integration site resulting in two ends, linking the another nucleic acid having two ends to the ends of the opened (cut) integration site, respectively, and optionally further processing the nucleic acid comprising the insert to obtain an error-free copy. A particular integration site is a cloning site, optionally a multi-cloning site, in a particular embodiment having restriction sequences. Insertion into an integration site is also possible.

“Tag free” protein expression of a gene is an expression process wherein the gene of the protein expressed is so designed that the expressed protein is free of any artificial peptide tag sequence covalently linked to the amino acid sequence of the protein expressed. A tool used herein to provide tag free expression is a bidirectional promoter.

“Genomic integration sequences” flanking the expression unit are sequences useful for integration of the expression unit into the genome of a host, preferably a mammalian host.

“Terminal repeats” are DNA genomic integration sequences flanking the expression unit.

“Inverted terminal repeats” (ITRs) are DNA genomic integration sequences flanking the expression unit, which, by the effect of a transposase, are capable of integration of the expression unit into the genome of a host, preferably a mammalian host.

A “silencer sequence” as used herein, in a specific meaning, is a sequence part (or segment) in the expression construct the expression product of which, either RNA or protein, prevent a gene from being expressed, in a preferred embodiment prevents expression of a given protein. For example a typical silencer sequence used in the present invention is a DNA segment, which is a construct which when operates provides an artificial microRNA (miRNA) which blocks or inhibits expression of a given protein.

“Bidirectional promoter” is a promoter, which is capable of driving protein expression in both direction from the DNA, in particular the expression cassette the promoter is present in; consequently the promoter has two sides, a first side (typically called an A side) and a second side (typically called a B side). The two sides are to be differentiated at the first place functionally, while structurally may overlap. “Bidirectional expression” is a process when the promoter drives expression from both sides. With particular and non-binding terminology it may be understood that the bidirectional promoter drives two expression units: a first expression unit wherein the first side operates as a promoter and a second expression unit in which the second side operates as a promoter. Each expression unit may have every feature an expression unit typically has, including a gene expressed with start and stop codons, untranslated regions, optionally introns and optionally further regulatory sequence(s).

“Balanced” bidirectional expression is a process when the level of expression from the two sides of the promoter is synchronized, in particular conformed, in particular the levels are similar or essentially the same. In a particular embodiment the ratio of the expression levels of the transcripts expressed from the first and second sides is between 0.1 and 10, e.g. between 0.6 and 2, preferably between 0.9 and 1.1, more preferably between 0.95 and 1.05, and the expression levels are in a physiological range of expression.

“Driving protein expression” means that a promoter sequence controls, including initiating expression of a protein coding DNA during which the DNA is translated into an mRNA sequence; a promoter is typically under a regulation and also largely defines the level of expression.

A “selectable marker gene” is a gene which, when expressed in a cell, provides a trait which is useful for selection of the cell; typically, the cell is capable of proliferating under conditions which inhibit proliferation of other cells or conditions leading to their death.

A “positive selectable marker gene” is a gene which, when expressed in a transfected cell, provides selective growth advantage to said cell over cells under the same conditions but lacking expression of said positive selectable marker gene. The conditions may include those which are specifically adapted to the “positive selection”, e.g. exposing the cells to an effect, e.g. a physical or chemical effect which impairs growth in cells which do not have the expression of said positive selectable marker gene thereby providing selective advantage to those which have. Typically, during the positive selection, a compound is added to the cells which compound impairs growth of cells lacking expression of said positive selectable marker gene and which impairment is antagonized by said expression. Alternatively, conditions may include removal, e.g. withdrawal of a compound from the environment of the cells typically lacking the expression of the positive selectable marker gene, the growth of which is impaired in lack of said compound, whereas cells having and expressing said marker survive and grow under such condition.

An example for positive selectable marker gene is the Fah section marker which provides growth advantage to cells over cells lacking said marker (e.g. Fah^{-/-} cells) in the absence of a 4-Hydroxyphenylpyruvate dioxygenase (HPPD) inhibitor e.g. nitisinone (NTBC).

A “visible marker gene” is a gene which, when expressed in a cell, provides or allows the production of a detectable visible signal. In particular embodiments, the “visible marker gene” encodes a protein which, when expressed and brought into appropriate state or under appropriate conditions, produces a visible signal. A “fluorescent marker gene” is a visible marker gene which, when expressed in a cell, emits a detectable fluorescent signal. Preferably the “fluorescent marker gene” encodes a fluorescent protein the fluorescence of which is detectable in the cells comprising said protein.

A “gene of interest” as used herein refers to a nucleic acid of interest encoding a protein of interest to be expressed in the target transduced cell. While the term “gene” may be used, this is not to imply that this is a gene as found in genomic DNA and is used interchangeably with the term nucleic acid encoding a protein. Generally, the nucleic acid of interest provides suitable nucleic acid for encoding the protein of interest and is operably linked to expression control sequences to effectively express the protein of interest in the target cell. The gene of interest may comprise cDNA or DNA and may or may not include introns but generally does not include introns.

A gene of interest as used herein is typically a gene the effect of which is to be examined in defined environment, e.g. in a transgenic cell or tissue or organ or animal.

“Mutation frequency” in terms of tumors as used herein means a ratio of tumors in which a given mutation can be found.

“Penetrance” of a tumor means the level or ratio of cells from which tumor development occurs if a given number of cells are taken in which a given driver mutation is present. In alternative wording, penetrance refers to the likelihood that a clinical condition will occur when a particular genotype is present or describes how likely it is that a person who has a certain disease-causing mutation (change) in a gene will show signs and symptoms of the disease. Thus, complete penetrance means that every person who has the mutation will show signs and symptoms of the disease. As an example of high penetrance tumors, from cells carrying a Ras mutation a very high percentage tumor develops if the cells survive. A low penetrance means that only a low ratio of cells carrying the driver mutation develops into a tumor.

“Sequence identity” as used herein relates to a definition of sequence identity in two aligned sequences as calculated in a method accepted in bioinformatics, e.g. a pairwise identity with another (reference) sequence for the whole sequence (if not indicated otherwise) or for a corresponding part thereof.

A sequence or part of a sequence (also called segment) “corresponding” to another sequence or part thereof is interpreted herein based on sequence alignment as this term is used in bioinformatics, and the corresponding sequences or sequence parts are those which are aligned with each other with any sequence alignment tool accepted in the art. While different methods and different algorithms are known and applied, any of such sequence alignment tool may be applicable in the present application which provides a meaningful result based on sequence similarity.

In particular, the alignment may be global alignment (calculated using e.g. the Needleman-Wunsch algorithm) or local alignment (calculated using e.g. the Smith-Waterman algorithm) [Wing-Kin., Sung (2010). Algorithms in bioinformatics: a practical introduction. Boca Raton: Chapman & Hall/CRC Press. pp. 34-35. ISBN 9781420070330. OCLC 429634761].

“Comparing” two levels is understood herein to include a comparison of quantities expressed in numerical values characterizing said levels to establish which is higher or lower, or establishing a difference or establishing a ratio of the levels, or values derived from the levels, optionally completed with other mathematical procedures as the quantification or calculation method requires.

The terms “comprises” or “comprising” or “including” are to be construed here as having a non-exhaustive meaning and allow the addition or involvement of further features or method steps or components to anything which comprises the listed features or method steps or components.

The expression “consisting essentially of” or “comprising substantially” is to be understood as consisting of mandatory features or method steps or components listed in a list e.g. in a claim whereas allowing to contain additionally other features or method steps or components which do not materially affect the essential characteristics of the use, method, composition or other subject matter. It is to be understood that “comprises” or “comprising” or “including” can be replaced herein by “consisting essentially of” or “comprising substantially” if so required without addition of new matter.

### Abbreviations

Afp: alpha-fetoprotein
AIM2: absent in melanoma 2
amiR: artificial microRNA
CDS: coding sequence
cGAS: cyclic GMP-AMP synthase
EEF1A1: eukaryotic translation elongation factor 1 alpha 1
EGFP: enhanced green fluorescent protein
Fah: fumarylacetoacetate hydrolase
FACS: Fluorescent Activated Cell Sorting
GOI: gene of interest
Gpc3: glypican-3
HADHA/B: hydroxyacyl-CoA dehydrogenase trifunctional multienzyme complex alpha and beta subunits
HCC: hepatocellular carcinoma
IHC: immunohistochemistry
ITR: inverted terminal repeat
KO: knock out
LINE1 (L1): long interspersed nuclear element 1
Luc: firefly luciferase
miR: microRNA
NTBC: 2-(2-nitro-4-trifluoromethylbenzoyl)-1,3-cyclohexanedione (Orfadin®, Nitisinone)
ORF1: Open Reading Frame 1 of LINE1
ORF2: Open Reading Frame 2 of LINE1
PB: piggyBac transposon
PCR: Polymerase Chain Reaction
Rpl27: ribosomal protein L27
RT-qPCR: quantitative reverse transcription PCR
qPCR: quantitative PCR
SB: Sleeping Beauty transposon
shRNA: short hairpin RNA
TLR9: Toll-like receptor 9
WT: wild type

### BRIEF DESCRIPTION OF THE FIGURES

FIG. 1—L1 retrotransposition cycle and its regulators in somatic cells. A Scheme of the classical retrotransposition mechanism of L1. Retrotransposition starts with the transcription of the progenitor L1 copy into a full length bicistronic mRNA in the cell nucleus. The L1 RNA transcript is transported to the cytoplasm, where the ORF1p and ORF2p proteins are synthesized and the L1 RNP is formed. The L1 RNP is then transported back to the nucleus and the L1 DNA copy formed by reverse transcription is integrated into a new genomic locus. B Outline of the main defense mechanisms of somatic cells to prevent L1 activation. Inhibition at the chromatin and DNA levels occurs by decreasing chromatin availability due to heterochromatinization and DNA methylation and with the participation of transcription factors. L1 is also inhibited by microRNA pathways by the cleavage of its transcript. In the cytoplasm antiviral proteins interact with L1-RNP leading to stress granule formation and L1-RNP degradation. Integration into the genome is impeded by members of the deaminase protein family and factors of DNA repair. Intracellular sensors that detect the presence of L1 components might stop the division of L1-expressing cells by regulating the cell cycle. Light gray arrows, L1 promoter; pA, polyadenilation signal; RNP, ribonucleoprotein.

FIG. 2—Outline of the somatic L1 activity assay. Transposon-based somatic gene delivery of the ORFeus reporter system into the liver of Fah −/− mice, animal treatments and assay quantification strategies. A Schematic representation of the piggyBac (PB) transposon-based cloning platform for the generation of ORFeus reporter constructs and animal treatments. Black (larger and longer) arrows, PB transposon inverted terminal repeats; smaller and shorter black arrows, promoters; light grey box (between Fah and mCh), Thoseaasigna virus 2A (T2A) peptide. Fah, fumarylacetoacetate hydrolase; amiR, artificial microRNA; NTBC, 2-(2-Nitro-4 trifluoromethylbenzoyl)-1,3-cyclohexanedione. B Structure and function of the autonomous and non-autonomous ORFeus-type L1 retrotransposition reporter constructs. C Assay quantification strategies to measure EGFP expression in the liver of mice carrying the ORFeus reporter. 1. After perfusion and hepatocyte isolation from the livers of treated mice, EGFP positive cell content is measured by FACS. 2. Intronless ORFeus copies can be detected by qPCR after DNA isolation from the liver of treated animals. 3. C-terminal FLAG-tagging of EGFP provides the possibility of IHC-based detection of full length EGFP with an anti-FLAG antibody. White arrows, promoters; pA, poly(A) addition signal; black half arrows, qPCR primers; white arrows, promoters; IHC, immunohistochemistry.

FIG. 3—Demonstration of successful hepatic expression of the ORFeus reporter by IHC. A Fah and L1-ORF1p immunostainings of liver sections from age matched WT mice reveal evenly distributed Fah immunopositive cells, while L1-ORF1p immunopositivity was not detectable. B Fah and L1-ORF1p immunostainings of liver sections from Fah −/− mice bearing the ORFeus reporter, after 3 months following hydrodynamic injection and NTBC withdrawal. Total visible liver areas are immunopositive for both Fah and L1-ORF1 proteins. The staining pattern indicates negative selection against L1-ORF1 (i.e. smaller clusters of highly immunoreactive (dark brown) cells and more extensive regions of lower positivity (light brown)). The ORFeus reporter variant used is shown at the bottom of the panel. Shorter and thicker arrows, promoters; gray box between the Fah and mCh boxes, Thoseaasigna virus 2A (T2A) peptide; pA, polyadenilation signal; thin black arrow, intron; TF, Tf monomer; Fah, fumarylacetoacetate hydrolase; WT, wild-type; Scale bars, 100 μm.

FIG. 4—Stereomicroscopic macrovisualization of EGFP positive hepatocyte colonies in the liver of Fah −/− mice carrying the ORFeus reporter construct. A Fluorescence stereomicroscopic images of the liver of Fah −/− mice carrying the ORFeus reporter construct without drug treatment. B FICZ treated animals show more EGFP fluorescent colonies in their liver as compared to the non-drug-treated controls. C Enlarged area indicated by dashed line box in B demonstrates EGFP positive colonies in higher magnification.

FIG. 5—Stereomicroscopic macrovisualization of EGFP autofluorescence in the liver revealed that MeIQx (n=9) and FICZ (n=7) treated animals exhibit a higher number of EGFP fluorescent hepatocyte colonies in their liver as compared to the non-drug-treated controls (n=10). The numbers of EGFP positive hepatocyte colonies per animal were plotted on a box diagram.

FIG. 6—Decitabine is a weak inducer of somatic L1 retrotransposition. A Monitoring the amount of intron-free EGFP copies produced during retrotransposition of the ORFeus reporter. Liver samples were collected from ORFeus reporter bearing Fah −/− mice three months after Decitabine treatment. Non-drug-treated Fah −/− mice, carrying the ORFeus reporter served as controls. Samples were tested using intronless EGFP qPCR assay. Results were normalized to the measurements of the olfactory receptor 16 (Olfr 16) gene as input control and data were presented as the mean±SD (n=2). B Determining the number of EGFP-positive cells carrying ORFeus retrotransposition events by FACS. For this, WT, non drug-treated ORFeus carrying and Decitabine-treated ORFeus carrying Fah −/− mice were subjected to liver perfusion and hepatocyte isolation. Subsequent FACS measurement of 1 million hepatocytes from each sample showed higher number of EGFP-expressing cells in the Decitabine-treated group compared to controls. Data were presented as the mean±SD (n=2) in the case of Decitabine-treated group and n=1 for control groups. WT, wild-type; RT-qPCR, quantitative reverse transcription PCR; FACS, Fluorescence-activated Cell Sorting.

FIG. 7—Expression vector with full ORFeus element and without amiR

FIG. 8—Expression vector with ORF1, with TF monomer, and without amiR

FIG. 9—Expression vector with ORF1, with no amiR, and without TF monomer

FIG. 10—Expression vector with ORF1, with TF monomer and with amiR-mP53/1 element

FIG. 11—Expression vector with a Flag tag at the end of EGFP exon 2 and without amiR

### DETAILED DESCRIPTION OF THE INVENTION

The present invention provides an opportunity to assess unconventional genotoxic effect of chemicals in somatic cells of mice. For example, a chemical for which a tumor-induction effect is suspected but the mechanism is not known, can be tested for an indirect mutagenic effect via the activation of L1 retrotransposons. Also the possibility of L1 retrotransposition as a mechanism can be excluded. Therefore, this innovative technology could be used in the field of toxicology, supporting chemical risk assessment toward toxicological endpoints not yet covered by known/standardized methods.

The present inventors have developed a technology platform that is suitable for measurement of ORFeus reporter in somatic transgenic liver of model animals. The present inventors have developed a technology platform that allows the expression of either a protein or a complex transcript (e.g. ORFeus), even if it is not preferred (under negative selection) in the primary cells, at the appropriate level in the whole liver cell population (approximately 100 million hepatocytes) of an experimental mouse. Thus, sustained expression of the ORFeus reporter in the mouse liver in vivo has been achieved.

Thereby the present model animals having somatic transgenic liver comprising the ORFeus reporter are useful to test any compound or effect for modulating, e.g. increasing L1 transposition activity.

The animals of the invention comprise in their genomes an expression cassette flanked by a pair of genomic integration sequences, said cassette comprising
a mammalian, preferably human bidirectional promoter, driving operably linked protein expression by two sides of the promoter, a first side and a second side,
a first expression unit, under the control of the first side of the promoter, said first expression unit comprising a positive selectable marker gene allowing, once expressed in the liver cells, positive selection of the cells,
a second expression unit, under the control of the second side of the promoter, comprising an ORFeus reporter element wherein said ORFeus reporter element comprises
   a gene encoding LINE1-ORF1 (L1-ORF1 or ORF1 in short) and optionally a further gene encoding LINE1-ORF2 (L1-ORF2 or ORF2 in short), and
   a retrotransposition reporter gene encoding a retrotransposition reporter protein
wherein
said ORFeus reporter element is transcribed from the second side of the promoter once the expression cassette is stably integrated into the genome of the transgenic liver cell and said ORF protein(s) is/are expressed, and
a retrotransposition reporter protein from said retrotransposition reporter gene is provided (i.e. expressed) only when the ORFeus reporter element is subject to retrotransposition in the genome of the transgenic liver cell.

The ORFeus-type reporters are well known in the art (Han and Boeke 2004). Such elements, when retrotransposed, produce a strong EGFP (or another marker) expression permanently in the given cell and its progeny.

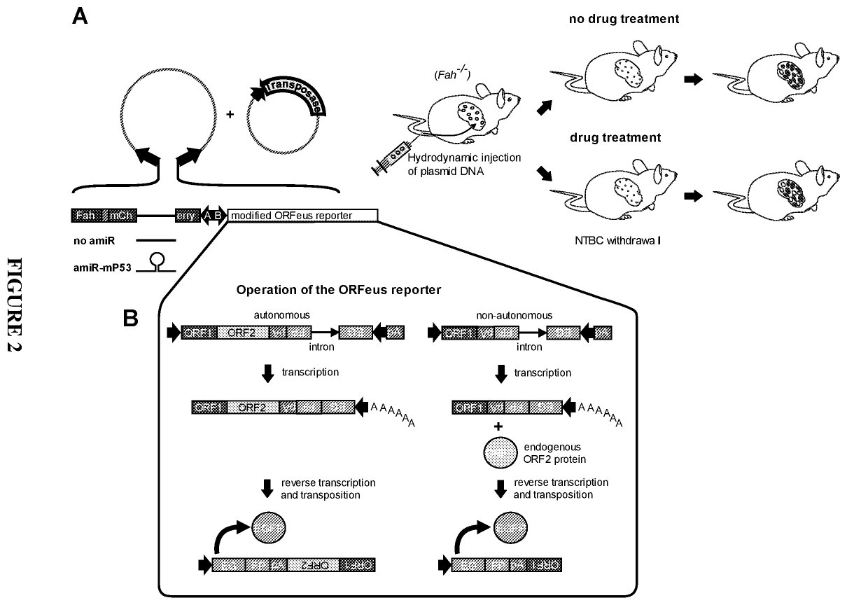

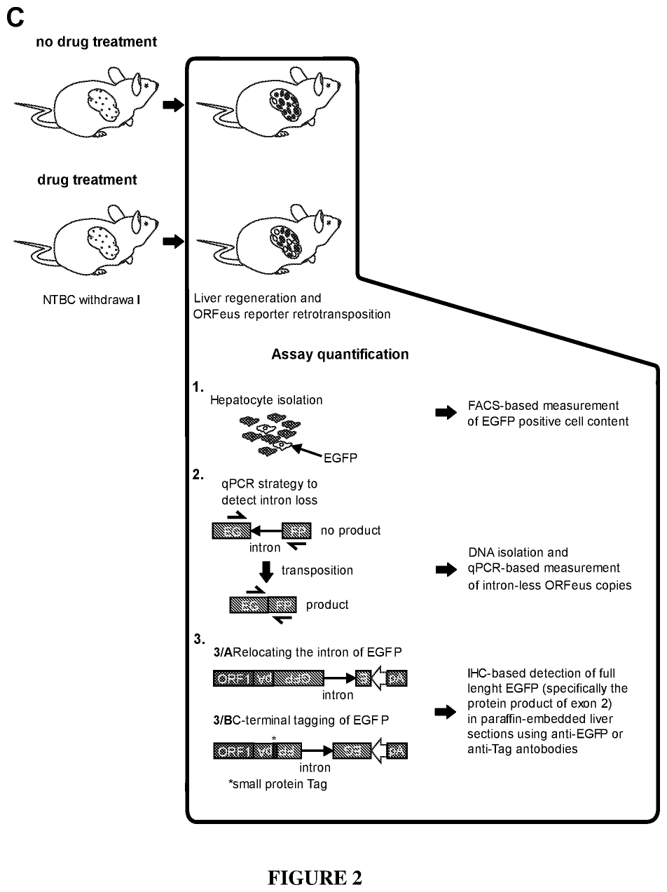

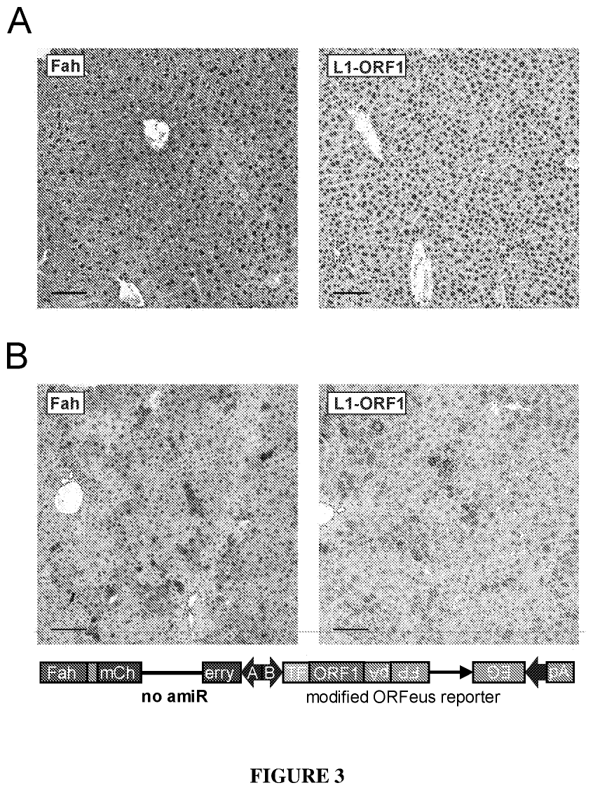

This is achieved by the removal of an intron by splicing from the retrotransposition detecting marker gene (e.g. EGFP) the orientation of which is opposite to the exons of the retrotransposition reporter gene e.g. EGFP, so that when the mRNA is transcribed from the antisense strand driven by the second side of the bidirectional promoter, the mRNA from the antisense strand of the reporter gene is spliced and the intron is removed whereas no reporter protein can be transcribed from this mRNA. Only when a reverse transcription and transposition event occurs due to the concerted effect of ORF1 and ORF2 proteins, the coding sequence of the retrotransposition reporter gene together with its own promoter is integrated into a site, different from the original one, of the liver cell genome, in the form of a DNA and the coding strand is restored. Thus, the retrotransposition reporter protein is expressed from this new site and a visible signal, preferably a fluorescent signal (e.g. in case of EGFP) is formed (see FIG. 2B). The cells with the visible signal, e.g. fluorescent cells, obtained them from the transgenic liver can be detected e.g. with FACS (see on FIG. 3.C/1).

In variant embodiments the expression of the retrotransposition reporter protein can be detected by qPCR. As shown on FIG. 3.C/2 the primers appropriately selected to bind to exon 1 and exon 2 respectively do not result in the desired product unless intron-less ORFeus copies are formed due to retrotransposition.

In the specific variant described in Example 2 and generalized in the first embodiment above, the detection of the ORFeus reporter does not allow for IHC staining-based readout, because a large part of the EGFP protein encoded by EGFP exon 1 (see FIG. 2B) is always present in cells expressing the reporter even without undergoing ORFeus retrotransposition. The commercially available EGFP antibodies tested so far by the present inventors are all able to bind to this EGFP fragment. This is due to the large size of exon 1 and the fact that the EGFP region encoded by it contains the most immunogenic epitopes. The transcript initiated from the EGFP promoter carries the EGFP exons and the intron, as the intron is not excised from the mRNA from this direction. Because the intronic region contains an in-frame stop codon just a few base pairs after the end of exon 1, the large EGFP fragment expressed from exon 1 carries only an additional 3-amino acid tag LLR* (herein * indicates a stop codon) which is provided by the intronic region and is not part of the original EGFP sequence.

The present inventors develop an immunohistochemistry (IHC) staining-based readout for the assay allowing selective immunostaining for the full-length EGFP protein, which is only produced in cells after retrotransposition of the ORFeus reporter (FIG. 2B).

An antibody specific for the polypeptide encoded by exon 2 of EGFP should be used to avoid this obstacle of the IHC staining-based readout. To achieve this, several possible alternatives are under testing or will be tested.

One solution is to relocate the intron separating the EGFP CDS into two exons in a way that exon 2 will be larger and the polypeptide it encodes will carry the binding site for some known antibodies specific for EGFP.

Another solution is to incorporate small peptide tags at the end of the EGFP exon 2, which will allow the use of antibodies specific for the particular tag.

Thus, in a further embodiment a tag is attached to the second exon of the retrotransposition reporter protein. The larger first exon of EGFP gets translated even without retrotransposition of the ORFeus reporter, so that it is present in all cells the genome of which harbors the expression construct. In this embodiment the second exon carries a tag which can be recognized by a specific antibody and thus the antibody detects the full length reporter protein only.

A preferred example is a Flag-tag (Flag-tag; DYKDDDDK, SEQ ID NO: 29) and an anti-Flag-tag antibody which could thus specifically detect this full length EGFP protein bearing an accordingly positioned Flag-tag in paraffin-embedded sections. [Einhauer A, Jungbauer A (2001). “The FLAG peptide, a versatile fusion tag for the purification of recombinant proteins”. Journal of Biochemical and Biophysical Methods. 49 (1-3): 455-65.] Another preferred example is the application of a V5-tag (V5-tag; GKPIPNPLLGLDST, SEQ ID NO: 30) and an anti-V5-tag antibody (Schutt, Hallmann et al. 2020).

The present invention allows, to the best of the inventors' knowledge to the first time, measurement of LINE1 (L1) retrotransposition activity in an in vivo somatic transgenic organ of an experimental animal.

The importance of the invention of the present inventors is particularly emphasized by the fact that the measurement of somatic L1 activity in germline-modified mouse models is problematic, as L1 elements are active in germ cells and early embryos, and thus all L1 reporters are activated early in development. Unless the sensitivity of such a germline-modified reporter mouse model is greatly reduced, adult animals will carry L1 reporter transpositions generated at earlier developmental stages throughout the body. As a consequence, they would be unsuitable for the study of somatic retrotransposition activity.

This problem has been overcome by the present invention.

Specifically, the measurement of somatic L1 retrotransposition and the elucidation of the chemicals that act on it are of particular importance because of their potential role in the development of sporadic cancers.

The liver can be efficiently targeted with naked plasmid DNA using a simple in vivo transfection procedure called hydrodynamic injection.

However, transgene expression rapidly declines in the liver following plasmid DNA delivery (Herweijer, Zhang et al. 2001). To improve the outcome of plasmid DNA delivery, the system can be supplemented with non-viral transposon-based chromosomal gene transfer. In the present examples the present inventors used PiggyBac (PB) transposon inverted terminal repeat (ITR) elements, as the PB transposon system is preferred for transporting relatively larger transgenes, and harnessed the selection pressure exerted in Fah deficient livers for Fah-expressing hepatocytes.

The present inventors have applied a somatic gene delivery technology enabling long-lasting and high-level transgene expression in the entire hepatocyte population of an animal liver.

The technology simultaneously allows the expression of either a protein or a complex transcript (e.g. ORFeus), and provide efficient silencing of any arbitrary target gene in the genome of a high number of transgenic cells in the liver of the animal.

The expression vector comprises a deficiency-complementing marker gene as a positive selectable marker and is useful for in vivo somatic transgenesis of the liver of an animal the cell of which are deficient in the trait provided by the marker gene. In a particular embodiment the present inventors harnessed the known selection pressure exerted in fumarylacetoacetate hydrolase (Fah) KO livers for Fah-expressing hepatocytes (Overturf, Al-Dhalimy et al. 1996). In this example the withdrawal of a drug, e.g. a 4-Hydroxyphenylpyruvate dioxygenase (HPPD) inhibitor e.g. nitisinone (NTBC) released the selection pressure generated by type I Tyrosinemia in the mouse liver. Lack of a HPPD inhibitor, e.g. NTBC results in a selective disadvantage for Fah KO cells whereas an advantage for the transgenic cells.

To link the expression of any gene of interest to the expression of the Fah selection marker gene, the inventors used a bidirectional promoter. As a particularly preferred example, the HADHA/B promoter, driving bidirectional and balanced, physiological range gene expression, was applied.

In a preferred embodiment, a silencer sequence is included in the same construct which comprises the ORFeus element and on which the positive selection marker is present. Thus all genetic features are jointly represented in all transfected cells, in a particular embodiment in all Fah corrected liver.

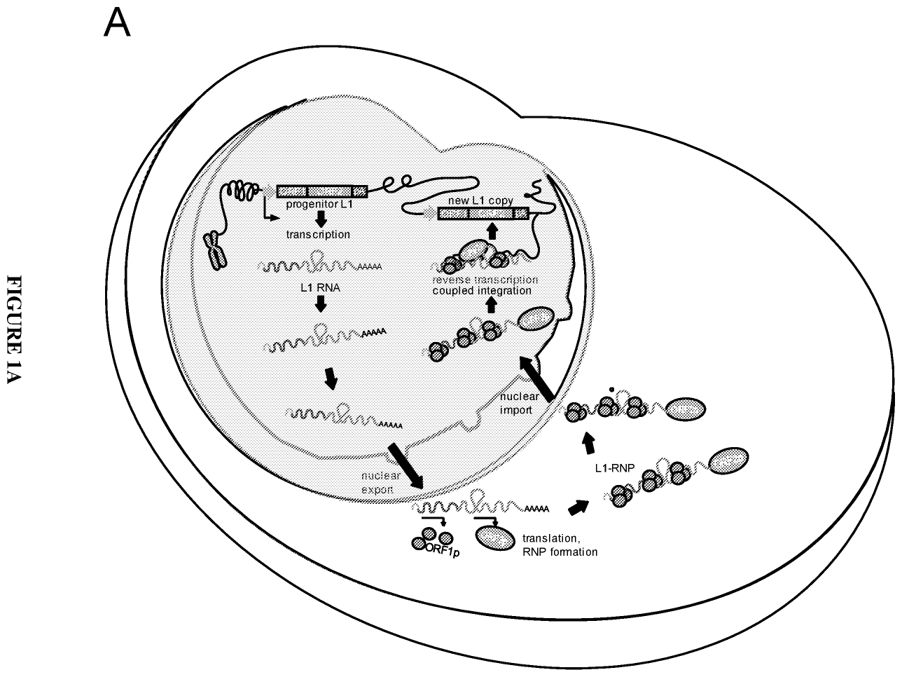

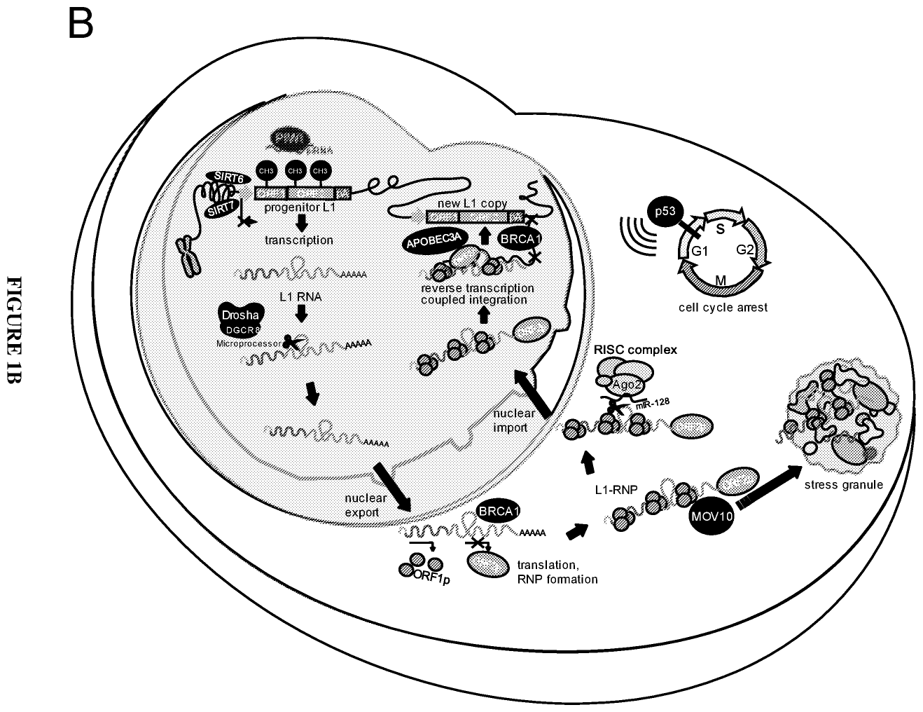

It is to be mentioned that the problem of the somatic delivery of the L1 reporter in order to provide a possibility to measure L1 retrotransposition in an in vivo setting has raised several difficulties. A part of difficulties arose from the fact that the somatic cellular defense systems that respond to L1 activity may eliminate the reporter-containing cells (FIG. 1B). In a particularly preferred embodiment these difficulties have been overcome by applying the following principles. First, the expression level of the ORFeus reporter has been set, to avoid an overly intense cellular response. The expression level should not be too low either, as this could not be sufficient to fulfil its reporter function. Second, the ORFeus expression must be linked to a positive selectable marker, otherwise the primary cells will not even tolerate the ORFeus expression necessary to fulfil its reporter function. As a particular advantage, if these conditions are met, the present technology provides the possibility of silencing any arbitrary gene in the mouse genome in a positive selectable marker and ORFeus linked manner, which gives the opportunity to further increase the sensitivity of L1 activity measurement. The applicability of amiR elements allows the weakening of certain somatic L1 defense lines (FIG. 1B). One example is to silence the Tp53. Thus, by using amiR elements, the inventors may sensitize the somatic L1 activity-measuring system. Exemplary defense lines are shown on FIG. 1B.

In the particularly preferred embodiments taught herein, this somatic gene delivery technology was used to express ORFeus-type L1 reporter elements linked to the Fah positive selection marker in the mouse liver. This arrangement allows efficient measurement of somatic L1 retrotransposon activity. The elements and steps of the complete measurement procedure are summarized in FIG. 2. First, ORFeus-expressing transposon and hyperPB transposase helper plasmids were co-delivered hydrodynamically into the liver of Fah deficient (Fah^{-/-}) mice, then the curative drug nitisinone (NTBC) (Overturf, Al-Dhalimy et al. 1996) was withdrawn (FIG. 2A). Immunohistochemical (IHC) investigations demonstrated that due to intensive multi-nodular repopulation after 3 months, virtually all hepatocytes were Fah- and L1-ORF1-positive in the treated livers (FIG. 3B). The mottled pattern of immunopositive cell clusters is due to the fact that colonies of a few hundred hepatocytes carrying different transposon integration events express Fah or L1-ORF1 proteins with slightly different intensities due to the different positions and copy numbers of transposons in their genomes. The more immunopositive (darker brown) cells form smaller clusters because divide less intensely and remain in smaller numbers during liver regeneration due to the negative selection pressure on L1-ORF1 protein expression. In contrast, when the presence of the same proteins was examined in untreated wild-type animals of the same age, striking differences were observed (FIG. 3A). As expected, the Fah immunopositivity in wild-type animals shows a nearly homogeneous pattern, reflecting the expression of the endogenous Fah gene. Remarkably, no L1-ORF1 immunopositive cells were found in the livers of wild-type animals, as indicated by the complete absence of brown signal in the sections. This is fully consistent with the previous finding of others that L1 protein expression is absent from normal somatic tissues (Rodic, Sharma et al. 2014). Thus, we conclude that our technology has enabled the L1-ORF1 protein, which is essential for the function of the L1 reporter, to be expressed extensively and stably in liver tissue of experimental mice, despite the negative selection pressure on it. The outline of the applied transposon construct and ORFeus variant is also depicted in the FIG. 3B.

It is worth noting that the present inventors have also tried somatic expression of the ORFeus reporter with the help of the Fah selection system using other promoters. However, these were not bidirectional, we then used a different method to try to link ORFeus reporter expression to the positive selection marker Fah. But in these cases, the experimental animals died and liver regeneration could not be achieved. We hypothesized that this was due to inappropriate (too high) levels of ORFeus reporter expression. This further demonstrates that the expression of L1-ORF1 and L1-ORF2 proteins in healthy somatic cells is highly contraselective and that the feasible expression level should be below a certain threshold. Such promoters, which we have unsuccessfully tried to apply to ORFeus expression in vivo in primary hepatocytes, were the CMV and CAGGS promoters.

In a particularly preferred embodiment, our technology platform also allows the silencing of any endogenous gene in hepatocytes by incorporating amiR elements into the transposon vector (FIG. 2A). This can be used to enhance the sensitivity of our somatic L1 activity assay.

For example, silencing of the Tp53 gene can attenuate the P53 L1 sensor (Ardeljan, Steranka et al. 2020) (FIG. 1B), without completely inactivating it. Such an effect is predicted to increase the tolerance of somatic cells to ORFeus reporter expression. We have developed three efficient Tp53 silencing amiR elements. Of these, amiR-mP53/1 capable to produce approximately 20% remaining Tp53 gene expression is currently being tested in our laboratory. Presumably, it will be able to increase the sensitivity of the somatic L1 activity measuring system. However, its application is not essential. Our results so far, at least in our in vivo system, are in conflict with the finding of the authors describing the role of the P53 sensor stating that wild-type retinal pigment epithelium-1 (RPE) cells undergo a TP53-dependent growth arrest in response to L1 (Ardeljan, Steranka et al. 2020). In contrast, ORFeus expressing mouse liver cells are able to regenerate the liver in the absence of p53 silencing (FIG. 3B).

Any other Tp53 specific amiR variant with a different target site, or any Tp53 specific amiR guide sequence incorporated into different miR backbone (e.g. miR155), may be equally effective. Possibly, attenuation of any other somatic L1 defense line besides P53 may also be effective.

### Variants of the ORFeus Reporter

More variants of the ORFeus reporter are currently being tested in our laboratory as detailed in the examples. Of the two proteins produced by the L1 retrotransposons, L1-ORF1 and L1-ORF2, L1-ORF2 can be omitted while L1-ORF1 is essential for the efficient operation of the reporter (summarised in FIG. 2B). L1-ORF1 is a high affinity RNA binding protein (Naufer, Furano et al. 2019) which, after translation, binds to its own mRNA and then facilitates further steps of retrotransposition, including the recruitment of L1-ORF2 to the L1 ribonucleoprotein complex (L1-RNP). The ORFeus reporter variants that do not express the L1-ORF2 protein have an advantage over the full-length reporter in that they do not function autonomously. In this case, the L1-ORF2 protein, which is also required for retrotransposition, is expressed from endogenous L1 copies released from the expression blockade (FIG. 1B). Thus, the non-autonomous system also reflects the expression status of endogenous L1 copies. Preparation of the constructs are described in the Examples.

The ORFeus reporter variant we currently use is derived from the pWA125 (Han and Boeke 2004, An, Han et al. 2006) construct. This ORFeus variant was generated by modifying the endogenous L1spa (Naas, DeBerardinis et al. 1998) mouse retrotransposon. From pWA125 we have transferred the ORFeus element into our expression system and performed the deletion of L1-ORF2.

Another element of the original ORFeus element is the Tf monomer region which functions as a promoter. The effect of the complete removal of Tf monomers is also currently being investigated in our laboratory. In this case, ORFeus expression will be driven solely by the HADHA/B promoter.

Beyond the potential use of other existing mouse or human ORFeus elements, an ORFeus element similar to the one in the pWA125 vector, which would work in our system, could be created from virtually any active mouse or even human L1 elements. Most human and mouse L1 sequences can be functionally exchanged (Wagstaff, Barnerssoi et al. 2011). L1 elements of other mammalian species have not been investigated in this respect, but it is assumed that the same may be true for L1 elements of related species such as rat or monkey. There are differences between active L1-ORF1 and L1-ORF2 sequences even at the amino acid level even within the same species, which is especially true for proteins from other species. Going further, sequence optimization could generate substantial sequence divergences when creating a new ORFeus variant.

Below the invention is further illustrated by examples. The skilled person will understand that these are not the only way to carry out the invention and therefore are non-limiting.

### EXAMPLES

### Example 1—Creating the Animals Used for Drug Testing

In these proof of concept studies the present inventors have used an L1-ORF2-free ORFeus and amiR-free construct variant (shown in FIG. 3B). First, the ORFeus-expressing transposon and the hyperPB transposase helper plasmid were co-delivered hydrodynamically into the liver of Fah^{-/-} mice, then the curative drug nitisinone (NTBC) was withdrawn (FIG. 2A). In those hepatocytes where hydrodynamic transfection is successful, the hyperactive transposase helper enzyme is likely to catalyse the “cut and paste” transposition reaction presumably leading to an integration into the host chromosomes (Skipper, Andersen et al. 2013). Upon NTBC withdrawal, this has resulted in intensive liver regeneration and multi-nodular repopulation and over 2-3 months the complete hepatocyte pool of the liver will carry the ORFeus reporter (FIGS. 2A and 3B).

### Example 2—Testing FICZ and MeIQx in the Somatic L1 Reporter Mouse Model

### 2.1 Visualization by EGFP Fluorescence

FICZ (6-Formylindolo[3,2-b]carbazole) is a derivative of tryptophan, and is a non-DNA-reactive non-genotoxic compound implicated in carcinogenesis (Rannug and Rannug 2018). Microbiota, both on the human skin and in the gut, can convert tryptophan to several metabolites including FICZ (Rannug and Rannug 2018). UVB radiation and H₂O₂ also spontaneously generate FICZ in human cells. FICZ is a known ligand of the aryl hydrocarbon receptor (AHR) that, among other things, plays a role in self-renewal and differentiation of stem/progenitor cells (Rannug and Rannug 2018).

17 Fah^{-/-} animals were injected hydrodynamically and after NTBC withdrawal, 7 animals were started on FICZ and 10 animals were kept without drug treatment as a control group. FICZ was administered by intraperitoneal (IP) injection at a dose of 5 mg/kg body weight twice weekly. Drug treatment regime started at the same time as NTBC withdrawal, thereby the ability of FICZ inducing somatic L1 retrotransposition in dividing primary hepatocytes during liver regeneration has been tested.

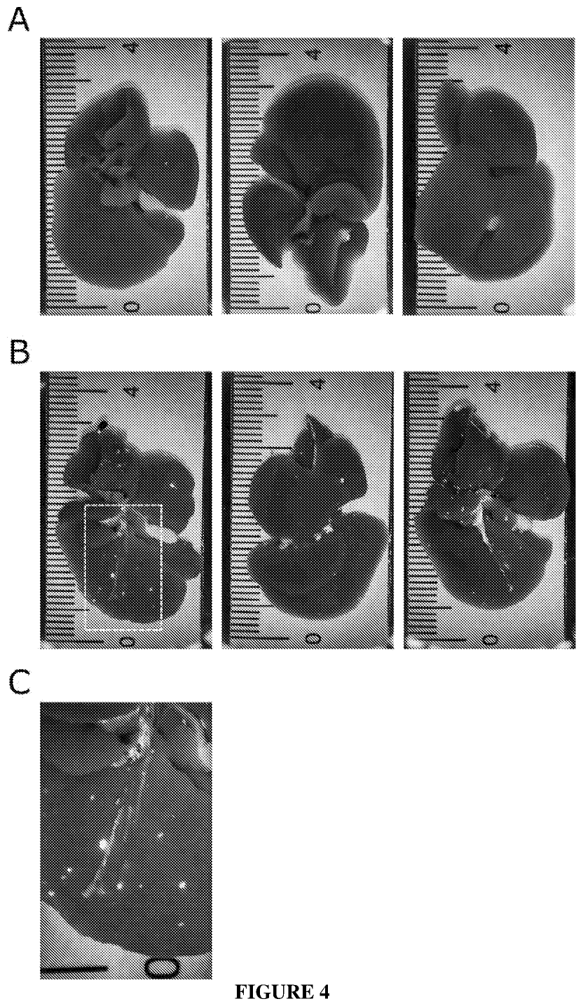

After 3 months following hydrodynamic injection and NTBC withdrawal mice were sacrificed. From each experimental group, livers were subjected to EGFP macrovisualization followed by DNA isolation. Macrovisualization of EGFP autofluorescence in liver revealed that FICZ-treated animals exhibit a higher number of stereomicroscopy detectable EGFP fluorescent (ORFeus retrotransposition bearing) hepatocyte colonies in their liver as compared to the non-drug-treated controls (FIG. 4). Thus, it has been demonstrated that the assay is useful to identify compounds that induce somatic L1 retrotransposition, bringing us closer to elucidating the underlying causes of sporadic cancers.

It is worth noting that forced expression of the ORFeus reporter also induced ORFeus retrotransposition events in non-drug-treated control animals. This is evidenced by the appearance of low number of EGFP-positive hepatocytes in control animals. Based on all this, it can be assumed that defensive mechanisms against somatic L1 activity cannot provide complete protection against L1 retrotransposition if the dominant expression of L1 elements becomes possible, for example due to epigenetic disorders.

### 2.2 Summary of EGFP Fluorescence Monitoring Data

An experiment similar to the one described in Example 2.1 has been carried out with the food-borne carcinogen MeIQx (2-Amino-3,8-Dimethylimidazo[4,5.f]Quinoxaline) a genotoxic heterocyclic amine.

9 Fah^{-/-} animals were injected hydrodynamically and after NTBC withdrawal, 9 animals were started on MeIQx. MeIQx was administered by IP injection at a dose of 5 mg/kg body weight twice weekly. Drug administration was started at the same time as NTBC withdrawal to test the ability of MeIQx for inducing somatic L1 retrotransposition in dividing primary hepatocytes during liver regeneration. After 3 months following hydrodynamic injection and NTBC withdrawal mice were sacrificed. Livers were subjected to EGFP macrovisualization followed by DNA isolation.

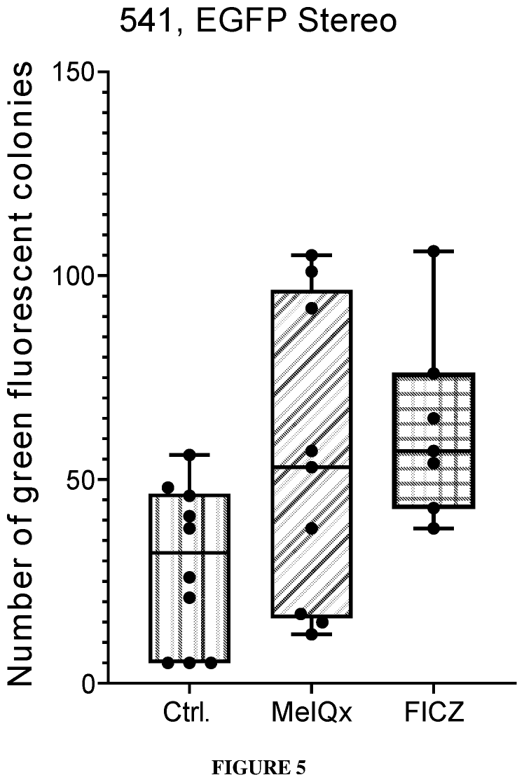

The results of EGFP macrovisualization were summarized in FIG. 5 for the control, FICZ-treated and MeIQx-treated experimental groups. The number of EGFP-positive hepatocyte cell colonies detectable by stereomicroscopy in the liver of each animal was counted and per-animal values were plotted on a box diagram. Analysis of the results revealed that MeIQx-treated animals also exhibited a higher number of EGFP fluorescent (ORFeus retrotransposition bearing) hepatocyte colonies in their liver as compared to the non-drug-treated controls (FIG. 5). This potentially means that DNA-reactive genotoxins, such as MeIQx, may also be able to activate somatic L1 retrotransposition.

### Example 3—Alternative Detection Methods

For evaluating alternative detection methods we investigated the outcome of Decitabine (5-aza-2′-deoxycytidine) treatments in the assay of the invention. 10 Fah^{-/-} animals were injected hydrodynamically and after NTBC withdrawal, 5 were started on Decitabine and 5 animals were kept without drug treatment as a control group. Under our current drug treatment regime administration of drugs starts at the same time as NTBC withdrawal. Based on our preliminary results Decitabine is a weak inducer of somatic L1 retrotransposition. This is in line with previous observations, since it is a hypomethylating agent that can reactivate silenced genes (Jabbour, Issa et al. 2008). It can thereby induce global hypomethylation on endogenous L1 copies (FIG. 1B), thus promoting the expression of L1-ORF2 from endogenous L1 copies, which is required for the activation of the non-autonomous ORFeus reporter.

In order to better evaluate the outcome of the assay, several measurement procedures suitable for obtaining quantitative results are being set up in our laboratory (summarised in FIG. 2C).

3.1 Detection by SYBR Green-Based qPCR Measurements

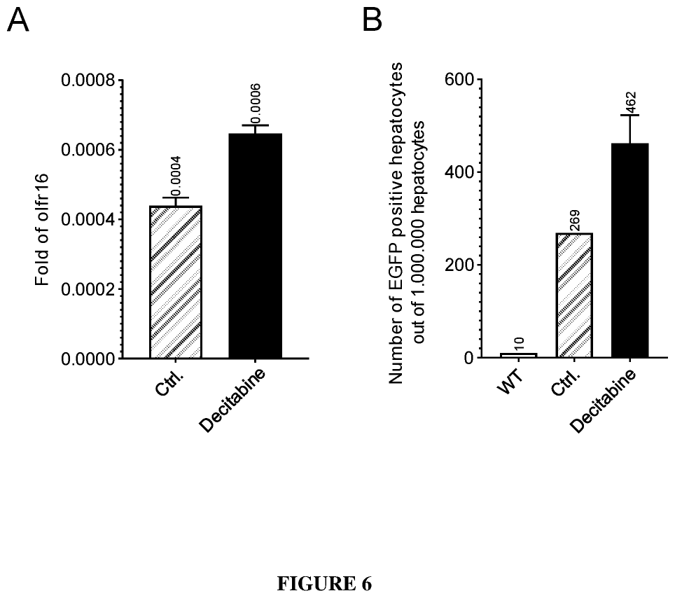

Multiple qPCR-based methods have already been published offering the possibility to measure the amount of intron-free EGFP copies produced during retrotransposition of the ORFeus reporter (Mita, Sun et al. 2020). Based on these published methods, we have also started to quantify our results. Our SYBR Green-based qPCR measurements so far confirmed that Decitabine is a weak inducer of somatic L1 retrotransposition (FIG. 6A).

### 3.2 Detection by FACS Measurement

Determining the number of EGFP-positive cells carrying ORFeus retrotransposition events by FACS also seems to be a viable detection method. To test this approach, 2-2 animals from Decitabine-treated and control experimental groups were subjected to liver perfusion and hepatocyte isolation. Subsequent FACS measurement of EGFP positive hepatocytes so far also confirmed the results obtained with qPCR (FIG. 6B).

### 3.3 Detection by Immunohistochemistry (IHC) Staining

The current version of the ORFeus reporter does not allow for IHC staining-based readout, because a large part of the EGFP protein encoded by EGFP exon 1 (see FIG. 2B) is always present in cells expressing the reporter even without undergoing ORFeus retrotransposition. The commercially available EGFP antibodies appear to be able to bind to the EGFP fragment encoded by the large first exon containing the most immunogenic epitopes. The transcript initiated from the EGFP promoter carries the EGFP exons and the intron, as the intron is not excised from the mRNA from this direction. The intronic region contains an in-frame stop codon just a few base pairs after the end of exon 1. Due to that the large fragment of the EGFP protein encoded by exon 1 is present in all cells underwent successful PB transposon-based gene delivery. Consequently, selective detection of the full-length EGFP would require a monoclonal antibody that is specific for an EGFP epitope encoded by the second smaller EGFP exon.

In an example the present inventors relocate the intron separating the EGFP CDS into two exons in a way that exon 2 will be larger so that the polypeptide it encodes may carry epitopes for antibodies specific for this part of the EGFP. Thereby IHC staining with these antibodies will be able to detect full-length EGFP only following ORFeus retrotransposition.

The inventors also plan to incorporate small peptide tags (Flag, V5, etc.) at the end of the EGFP exon 2, which could also provide a possibility to quantify the results of the L1 activity assay.

A construct comprising the C-terminal Flag-tagging (DYKDDDDK, SEQ ID NO: 29) of the EGFP marker protein has been created. This would be useful because the larger first exon of EGFP gets translated even without retrotransposition of the ORFeus reporter, so that it is present in all cells underwent successful PB transposon-based gene delivery. Consequently, selective detection of the full-length EGFP would require a monoclonal antibody that is specific for an EGFP epitope encoded by the second smaller EGFP exon.

Unfortunately, such an antibody is not commercially available. The fluorescent full-length version of EGFP, which also contains the polypeptide encoded by the smaller second EGFP exon, appears only after ORFeus retrotransposition. An Anti-Flag-tag antibody could specifically detect this full length EGFP protein bearing an accordingly positioned Flag-tag in paraffin-embedded sections. With this method, cells carrying L1 retrotransposition events could be easily counted on sections using an AI-based image analysis pipeline.

An additional construct variant containing a V5-tag (V5-tag; GKPIPNPLLGLDST, SEQ ID NO: 30) has also been generated, which in combination with an anti-V5-tag antibody could also be used to selectively detect the full-length EGFP protein.

Systems for Tagging Including Epitope Tag Coding Sequences, Suitable for Preparation of Constructs Comprising Flag or V5 Tagged EGFP, as Well as Antibodies Specific for Said Tags are Available Among Others from Addgene, Proteintech, Abeam, APExBIO Etc. EXAMPLE 4—Options in the Experimental Setup

In this example administration of chemicals started 3 months after initiating liver regeneration. In this setting, the somatic L1 retrotransposition-inducing effect of the given drug is investigated after the termination of the intensive hepatocyte divisions. This treatment schedule will be applied using a somatic L1 activator molecule that is more potent than Decitabine, once identified with the present assay.

Successful multi-nodular repopulation (Overturf, Al-Dhalimy et al. 1996) is driven by the enormous regenerative potential of the liver (Lehmann, Tschuor et al. 2012). In mammals, the regenerative potential of the liver is required for successful adaptation to environmental challenges like toxic effects or changes in diet quantity/quality. Thus, some degree of liver cell division is part of normal human life as well. Nevertheless, we keep in mind that a treatment timing option when the liver is more settled, i.e. the intensive hepatocyte divisions have been terminated, will also be used.

### Example 4—Methods

### Variants of the ORFeus Reporter

Multiple variants of the ORFeus reporter have been created and tested. Of the two proteins produced by the L1 retrotransposons, L1-ORF1 and L1-ORF2, L1-ORF2 was in certain examples omitted while L1-ORF1 is essential for the efficient operation of the reporter (summarised in FIG. 2B). L1-ORF1 is a high affinity RNA binding protein (Naufer, Furano et al. 2019) which, after translation, binds to its own mRNA and then facilitates further steps of retrotransposition, including the recruitment of L1-ORF2 to the L1 ribonucleoprotein complex (L1-RNP).

The ORFeus reporter variant used in the present example is derived from the pWA125 (Han and Boeke 2004, An, Han et al. 2006) construct. This ORFeus variant was generated by modifying the endogenous L1spa (Naas, DeBerardinis et al. 1998) mouse retrotransposon. In addition to the inclusion of the reporter cassette in its 3′UTR region, sequence optimization was performed in the L1-ORF1 and L1-ORF2 coding sequence (CDS) region (Han and Boeke 2004) to avoid prematured polyadenylation a known characteristic of endogenous L1 elements. From pWA125 the ORFeus element transferred into our expression system and performed the deletion of L1-ORF2. The L1spa element belongs to the L1MdTfI L1 subfamily one of the 8 currently active mouse L1 subfamilies (L1MdAI, L1MdAII, L1MdAIII, L1MdGfH, L1MdGfHI, L1MdTfI, L1MdTfII and L1Md TfIII), whose members also carry Tf monomers. The Tf monomer region functions as a promoter. In certain construct the TF promoter has been omitted.

### Plasmid Construction

Empty pbiLiv-miR vector was synthesized and cloned in a pUC57 plasmid backbone by GeneScript. This encompasses the bidirectional promoter of the human hydroxyacyl-CoA dehydrogenase trifunctional multienzyme complex alpha (HADHA) and beta (HADHB) subunits. The HADHA side of the bidirectional promoter drives expression of the mCherry fluorescent marker gene, which is disrupted by a modified version of the first intron of the human eukaryotic translation elongation factor 1 alpha 1 (EEF1A1) to ensure intronic expression of the designed amiR structures (for gene silencing). Restriction endonuclease recognition sites (cloning site 1) were introduced into the EEF1A1 intron to clone amiR elements as follows: AgeI, XbaI, SacI, SalI. The mCherry coding sequence (CDS) is linked to the mouse fumaryl-aceto-acetate dehydrogenase (Fah) CDS by a T2A peptide to provide bicistronic expression. The transcription unit ends with a bGH polyadenylation signal. The HADHB side of the bidirectional promoter is flanked by an MCS (again a recognition sites or cloning site 2) followed by a bGH polyadenylation signal (i.e. transcription unit end).

The whole arrangement is flanked by the transposon inverted terminal repeats.

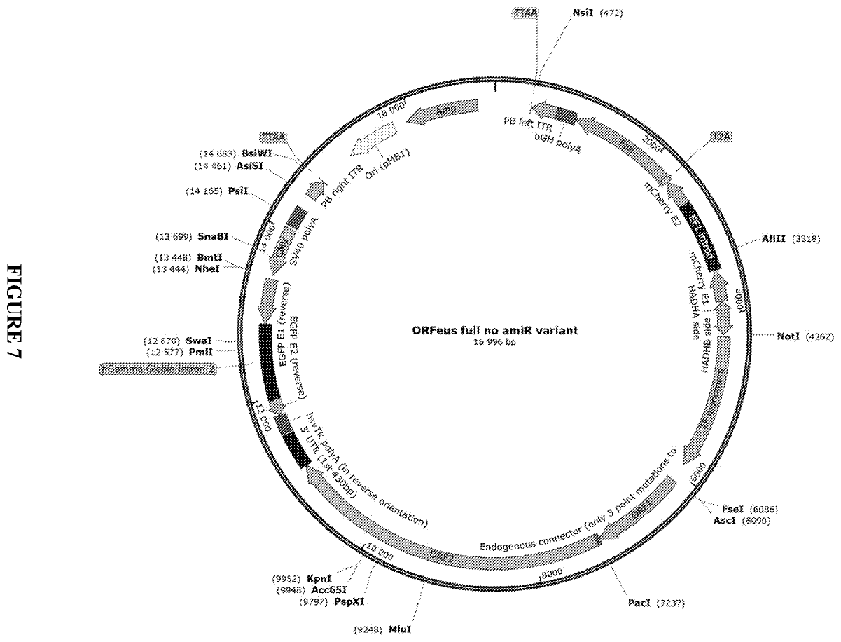

SEQ ID NOs 1 to 5 and FIGS. 7 to 11, respectively, show the variant expression vectors designed by the present inventors. The list of these vectors and elements including expression units on both sides of the bidirectional promoter (SEQ ID NOs 6 to 11) as well the EGFP expression unit for detection of the retrotransposition event (SEQ ID NO: 12) are listed in Table 1. Moreover, the individual components of the vectors are also listed in the sequence listing (SEQ ID NOs 13 to 24).

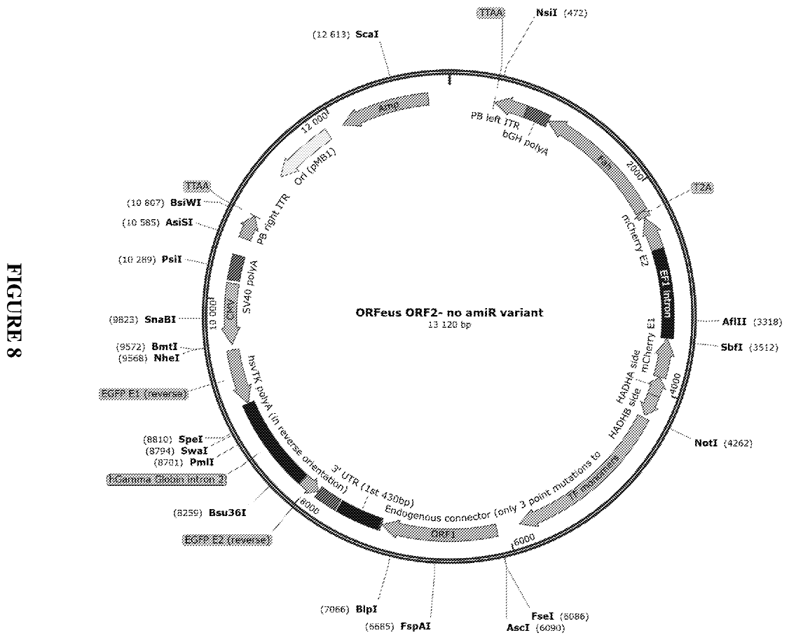

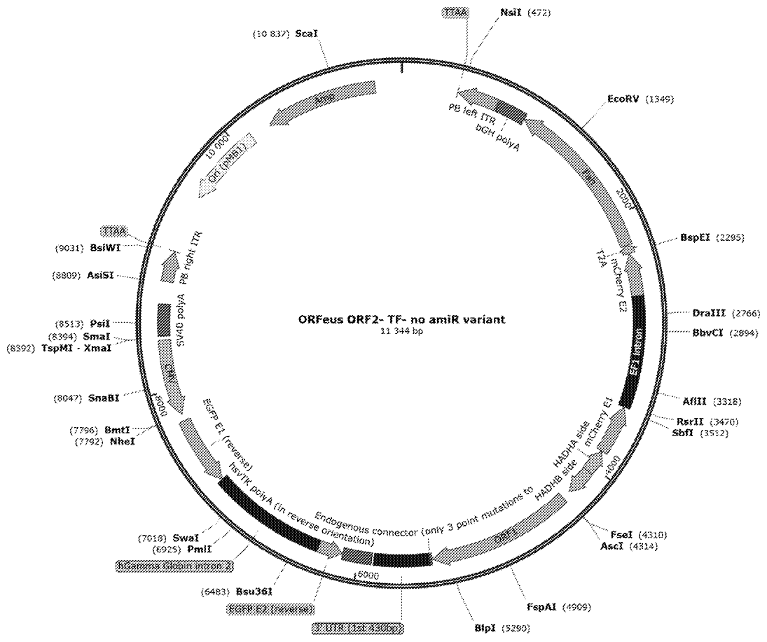

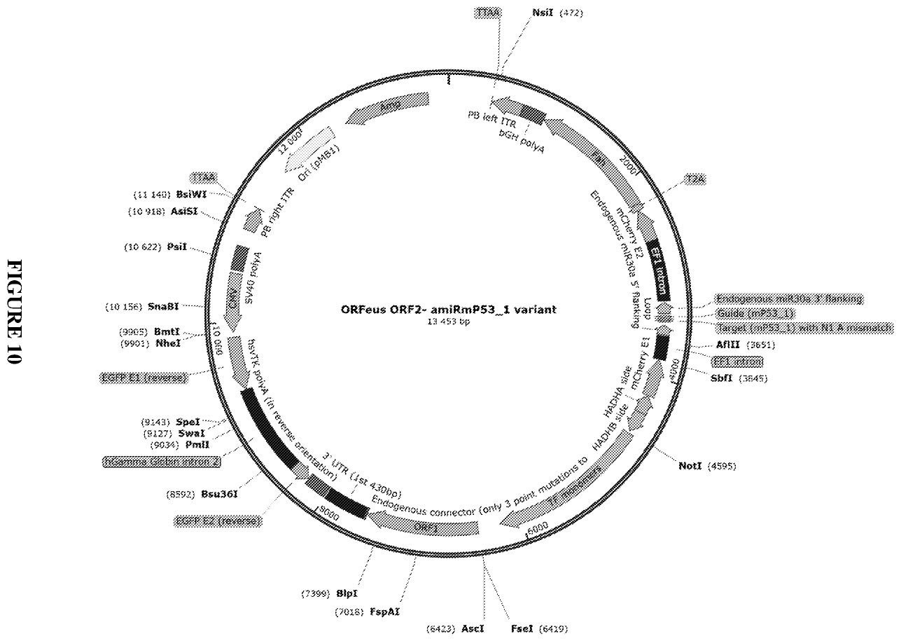

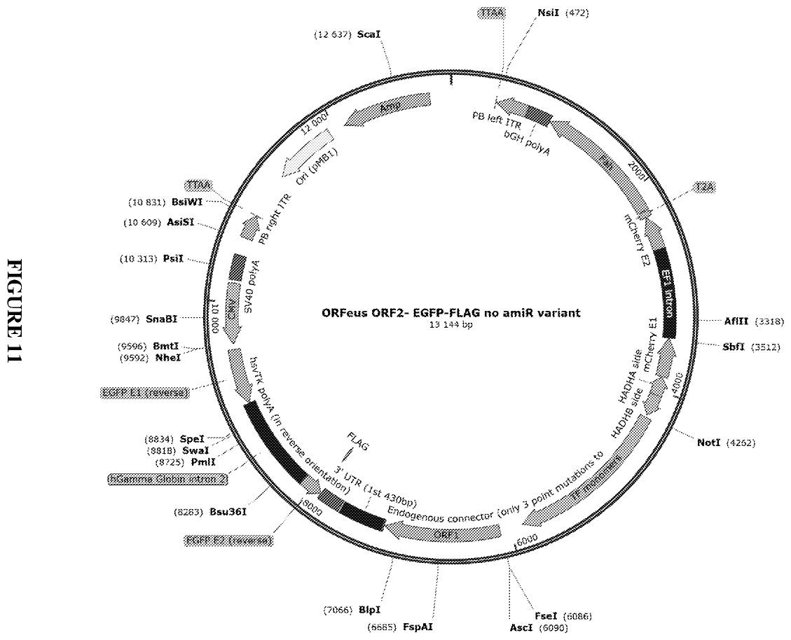

TABLE 1
The sequence listing comprises the following exemplary sequences

| | |
|---|---|
| SEQ ID NO: 1 | Expression vector with full ORFeus element and no amiR (FIG. 7) |
| SEQ ID NO: 2 | Expression vector with ORF1, TF monomer, and no amiR (FIG. 8) |
| SEQ ID NO: 3 | Expression vector with ORF1, with no amiR, and no TF monomer (FIG. 9) |
| SEQ ID NO: 4 | Expression vector with TF monomer and amiR-mP53/1 element (FIG. 10) |
| SEQ ID NO: 5 | Expression vector with a Flag tag at the end of EGFP exon 2 and no amiR (FIG. 11) |
| SEQ ID NO: 6 | First expression unit (HADHA side) of the expression vector with full ORFeus and no |
| | amiR (FIG. 7) |
| SEQ ID NO: 7 | First expression unit (HADHA side) with amiR-mP53 element (FIG. 10) |
| SEQ ID NO: 8 | Second expression unit (HADHB side) of the expression vector with full ORFeus and no |
| | amiR (FIG. 7) |
| SEQ ID NO: 9 | Second expression unit (HADHB side) with ORF1 and TF monomers (FIG. 8) |
| SEQ ID NO: 10 | Second expression unit (HADHB side) with ORFI and without TF monomers (FIG. 9) |
| SEQ ID NO: 11 | Second expression unit (HADHB side) with a Flag tag at the end of EGFP exon 2 |
| | (FIG. 11) |
| SEQ ID NO: 12 | EGFP-expressing element in the second expression unit (HADHB side) |
| SEQ ID NO: 13 | Amino acid sequence translated from EGFP exon 1 (E1) CDS* from SEQ ID NO 12 |
| SEQ ID NO: 14 | Amino acid sequence translated from EGFP exon 2 (E2) CDS from SEQ ID NO 12 |
| SEQ ID NO: 15 | Fumarylacetoacetate hydrolase (Fah) coding CDS |
| SEQ ID NO: 16 | Fah protein, amino acid sequence translated from of Fah CDS from SEQ ID NOs 6-7 and 15 |
| SEQ ID NO: 17 | HADHA/B promoter |
| SEQ ID NO: 18 | EF1 intron, the modified intron of human eukaryotic translation elongation factor 1 |
| | alpha 1 (EEF1A1) gene |
| SEQ ID NO: 19 | amiR-mP53/1 element |
| SEQ ID NO: 20 | ORF1 protein CDS (same as in SEQ ID NOs 8-11) |
| SEQ ID NO: 21 | ORF1 protein, amino acid sequence translated from of ORF1 CDS from SEQ ID NO: 20 |
| | and SEQ ID NOs 8-11 |
| SEQ ID NO: 22 | ORF2 protein CDS (same as in SEQ ID NO: 8) |
| SEQ ID NO: 23 | ORF2 protein, amino acid sequence translated from ORF2 CDS from SEQ ID NO 22 and |
| | SEQ ID NO: 8 |
| SEQ ID NO: 24 | TF monomers (same as in SEQ ID NOs 8-9 and 11) |

*CDS = coding sequence

The elements of the exemplary expression cassettes are also listed per elements and their reference in the sequence listing is given in Table 2 (see FIG. 9; the SEQ ID NOs are exemplary sequences as given in the sequence listing):

TABLE 2

| | | Exemplary nucleotides | |
|---|---|---|---|
| | Exemplary | in the expression units | |
| Expression cassette element | SEQ ID NO | SEQ ID NO: 6 | SEQ ID NO: 7 |
| piggyBac 5′ (left) ITR (Inverted Terminal | | 3307 to 3612 | 3640 to 3945 |
| Repeat) | | | |
| bGH polyA, polyadenylation signal (several | | 3071 to 3298 | 3404 to 3631 |
| alternative elements, e.g. SV40 polyA can also | | | |
| be used). | | | |
| Fah CDS (Fah coding sequence) | SEQ ID NO: 16 | 1811 to 3070 | 2144 to 3403 |
| | SEQ ID NO: 15 | | |
| T2A peptide for bicistronic translation of the | | 1748 to 1810 | 2081 to 2143 |
| mCherry and Fah CDSs (other elements like the | | | |
| F2A elem can also be used) | | | |
| mCherry E2 (the 2^{nd} exon of mCherry CDS). | | 1418 to 1747 | 1751 to 2080 |
| Ef1 intron (in an example two amiR cloning site | SEQ ID NO: 18 | 562 to 1417 | 562 to 795 and |
| can be found therein, see the map) | | | 1143 to 1750 |
| amiR cloning site 2 | | 847 to 866 | |
| amiR cloning site 1 | | 792 to 809 | |
| optional: amiR-mp53/1 element | SEQ ID NO: 19 | | 796 to 1142 |
| mCherry E1, (the 1^{st} exon of mCherry CDS). | | 184 to 561 | 184 to 561 |
| HADHA promoter start site | | 156 | 156 |
| HADHA/B promoter | SEQ ID NO: 17 | | |
| | | SEQ ID NO: 8 | SEQ ID NO: 12 |
| HADHB promoter start site | | 196 | |
| TF monomers | SEQ ID NO: 24 | 235 to 1834 | |
| ORF1 coding sequence | SEQ ID NO: 20 | 2056 to 3171 | |
| ORF2 coding sequence | SEQ ID NO: 22 | 3212 to 7057 | |
| 3′ UTR (1st 430 bp) | | 7058 to 7487 | |
| hsvTK polyA polyadenylation signal | | 7504 to 7727 | 2260 to 2483 |
| | | reverse | |
| EGFP exon 2 (E2) | SEQ ID NO: 14 | 7731 to 7916 | 2071 to 2256 |
| | | reverse | |
| hGamma Globin intron 2 | | 7917 to 8818 | 1169 to 2070 |
| | | | reverse |
| EGFP exon 1 (E1) | SEQ ID NO: 13 | 8819 to 9352 | 635 to 1168 |
| | | reverse | |
| CMV promoter | | 9402 to 9986 | 1 to 585 |
| | | reverse | |
| SV40 polyA polyadenylation signal | | 10017 to 10258 | |
| piggyBac 3′ (right) ITR (Inverted Terminal | | 10430 to 10666 | |
| Repeat) | | | |
| FLAG epitope tag | | SEQ ID NO: 11 | |
| | | 3858 to 3881 | |

An exemplary backbone vector is the pUC57 vector comprising a replication origin (Ori site) and a bacterial selection marker gene (e.g. Amp, ampicillin resistance site). (See an example for the complete vector with pUC57 vector backbone and with the expression cassette elements of Table 1 in SEQ ID NO: 1.) Actually any other typical backbone vector can be used.

The skilled person can compile any one of these vectors from the elements described above.

### Animal Care, Maintain and Drug Treatment

Mice were bred and maintained in the Central Animal House at the Biological Research Centre (Szeged, Hungary). The specific pathogen-free status was confirmed quarterly according to FELASA (Federation for Laboratory Animal Science Associations) recommendations. Mice were housed under 12 h light-dark cycle at 22° C. with free access to water and regular rodent chow. All animal experiments were conducted according to the protocols approved by the Institutional Animal Care and Use Committee at the Biological Research Centre. The used Fah mutant line, C57BL/6N-Fah^{tm1(NCOM)Mfgc/Biat}, is archived in the European Mouse Mutant Archive (EMMA) under EM:10787. Fah^{-/-} mice were treated with 8 mg/l Orfadin® (Nitisinone, NTBC) (Swedish Orphan Biovitrum) in drinking water. NTBC was withdrawn after hydrodynamic plasmid delivery. C57BL/6NTac wild-type mice were obtained from Taconic Biosciences. Dosing, scheduling, and the route of administration of all drugs and chemical compounds were determined according to the manufacturer's instructions and literature data. Decitabine was used at a dose of 1 mg/kg body weight dissolved in Phosphate-Buffered Saline (PBS). Administration was performed via the intraperitoneal (IP) route twice weekly (Lantry, Zhang et al. 1999). The body weight of the mice was monitored continuously. Vehicle (PBS, DMSO, corn oil) injections served as controls. The first dose was administered immediately after the NTBC withdrawal. The delayed drug administration setting 3 months past that (when the liver regeneration has been completed) is currently being tested.

### Hydrodynamic Tail Vein Injection

Plasmids for hydrodynamic tail vein injection were prepared using the NucleoBond Xtra Maxi Plus EF Kit (Macherey-Nagel) according to manufacturer's instructions. Before injection, we diluted plasmid DNA in Ringer's solution (0.9% NaCl, 0.03% KCl, 0.016% CaCl₂)) and a volume equivalent to 10% of mouse body weight was administered via the lateral tail vein in 5-8 seconds into 6-8 week-old mice. The amount of plasmid DNA was 50 μg for each of the constructs mixed with 4 μg of the transposase helper plasmid.

### Stereomicroscope Imaging

Pictures of whole mouse livers were taken with an Olympus SZX12 fluorescence stereozoom microscope equipped with a 100 W mercury lamp and filter sets for selective excitation and emission of GFP and mCherry.

### Liver Perfusion and Hepatocyte Isolation

Procurement of liver for hepatocyte isolation was done under sodium pentobarbital (Nembutal) (Sigma Aldrich) anaesthesia. The isolation of mouse hepatocytes was performed by a three-step collagenase perfusion. Briefly, mice were perfused through the vena cava superior with EGTA-containing Earle's balanced salt solution (EBSS) without calcium. Next, EGTA was washed out with EBSS, then the liver was perfused with EBSS containing 0.5 g/l Collagenase Type IV (Sigma Aldrich). Digested livers were removed and placed in ice-cold washing buffer (0.01 mM HEPES, 140 mM NaCl, 7 mM KCl, pH7.2). All subsequent steps were performed on ice. The liver capsule was opened to release the cells into the washing buffer by shaking. Cell suspension was filtered through a 100 μm filter to remove undigested tissue and debris. Cells were then centrifuged at 1000× rpm at 4° C. for 4 min. The pellet was resuspended in washing buffer and mixed with equal volume of Percoll solution (Sigma Aldrich). The suspension was centrifuged at 1000× rpm at 4° C. for 4 min. The pellet containing hepatocytes was washed with washing buffer and centrifuged at 1000× rpm at 4° C. for 4 min. Cell numbers were determined using a Burker chamber. Cell viability was determined by trypan blue exclusion test.

### FACS-Based Measurement of EGFP Positive Hepatocytes

Hepatocytes (2×10⁶/ml) prepared from mouse livers were suspended in PBS. Prior to measurement, cells were filtered through a 100 μm mesh filter to avoid cell clumps. EGFP fluorescence was analyzed on a BD FACSAria™ Fusion Flow Cytometer (Becton Dickinson) using standard flow cytometry. BD FACSDiva™ Software was used for analysis.

qPCR Strategies for Detecting Intronless EGFP Copies Generated During Retrotransposition

To measure the retrotransposition events of the synthetic L1 element we carried out genomic exon-exon junction qPCR analysis of the spliced, intronless EGFP using two different qPCR detection chemistry. SYBR Green based qPCR was done using PerfeCTa SYBR Green SuperMix (Quantabio). Cycling conditions were as follows: 95° C. for 7 min, 4 cycles of 10 s at 95° C., 15 s at 66° C. (−1° C./cycle, no acquisition), followed by 40 cycles of 5 s at 95° C., 10 s at 62° C. The following primers were used:

Olfr16-F:
(SEQ ID NO: 25)
GAGTTCGTCTTCCTGGGATTC
Olfr16-R:
(SEQ ID NO: 26)
TAATGATGTTGCCAGCCAGA
GFP-F:
(SEQ ID NO: 27)
AAGCAGAAGAACGGCATCAAGGT
GFP-EJ-R:
(SEQ ID NO: 28)
TGGTAGTGGTCGGCCAGCTGC

Probe-based qPCR detection was done as previously described (Mita, Sun et al. 2020) using PerfeCTa qPCR ToughMix (Quantabio). All qPCR reactions were performed on a Rotor-Gene Q instrument (Qiagen) in triplicates using 87 ng of gDNA. Analysis was carried out with the Rotor-Gene Q software (Qiagen). Relative changes in expression levels were calculated using the ΔCT method (Livak and Schmittgen 2001) SYBR Green and probe-based qPCR results were normalized to measurements of the Olfr16 and Rpl21 internal control genes, respectively.

Next Generation Sequencing (NGS) based detection of intronless EGFP copies generated during retrotransposition Quantitative measurement of retrotransposition events of the synthetic L1 element is also possible by NGS-based detection of spliced, intronless EGFP copies. In this setup, the use of EGFP-specific primers similar to the primers used in the qPCR procedure is required. With the difference that these EGFP primers must include the sequencing adapters used by Illumina. Amplicons prepared in this way can be sequenced on Illumina sequencers. During bioinformatic analysis, quantitative assay results can be obtained based on the NGS read count support of amplicons carrying intron-containing and intron-free EGFP sequences.

### Immunohistochemistry

Mice were sacrificed at 3 months post-injection. Livers were removed and fixed overnight in 4% formalin, then embedded in paraffin and cut into 5 μm sections. Immunohistochemistry was performed using the EnVision FLEX Mini Kit (DAKO). Antigen retrieval was done in a PT Link machine (DAKO). The primary antibodies used for immunohistochemistry are: rabbit polyclonal anti-FAH antibody (ThermoFisher Scientific, PA5-42049, 1:400), rabbit polyclonal anti-mCherry (GeneTex, GTX128508, 1:400), rabbit monoclonal anti-LINE-1 ORF1p antibody [EPR21844-108](Abcam, ab216324, 1:500), rabbit polyclonal anti-FLAG epitope tag antibody (Novus Biologicals, NB600-345, 1:400). Sections with the primary antibodies were incubated overnight. Secondary antibody polyclonal goat anti-rabbit-HRP (DAKO, P0448) was incubated for 30 min. Visualization was done with EnVision FLEX DAB+ Chromogen System (DAKO, GV825). After hematoxylin counterstaining for 5 min, slides were mounted and scanned with a Pannoramic Digital Slide Scanner (3D Histech).

### AI-Based Image Analysis Pipeline for Counting FLAG Immunopositive Cells

3D Histech generated images were processed using BIAS software. Pipeline was created for the analysis consisted of four major steps; 1.) pre-processing of the images, 2.) segmentation and 3.) feature extraction, 4.) cell classification using machine learning. In the pre-processing, non-uniform illumination was corrected using the CIDRE method. Deep learning segmentation method was applied to detect and segment individual nuclei in images. With segmentation post-processing, two additional regions were defined for each nuclei: 1.) a region representing the entire cell were defined by extending nuclei regions with maximum 5 μm radius so that adjacent cells did not overlap, and 2.) cytoplasmic regions were defined by subtracting nuclei segmentation from the cell segmentation. Finally, morphological properties of these three different regions as well as intensity and texture features from all channels were extracted (in total 228 features) for cell classification. We employed supervised machine learning to predict four different cell types: FLAG positive cells, FLAG negative cells, Immune cells and other cells or segmentation artefacts that can be considered Trash. These classes were manually selected based on their morphological characteristics. Cells with evenly distributed brown chromogen signal (anti-FLAG staining) across the whole cells were labelled as FLAG positive, whilst cells without chromogen staining were labelled as FLAG Negative. Cells with small and dark blue nuclei were considered as lymphocyte-like immune cells. Small segmented regions outside the tissue section were also classified as trash. For the training set, we annotated around 200 cells for each class from different tissue sections. Support Vector Machine (SVM) was trained with a radial basis function kernel commonly used for the multi-class cell phenotype classification. After training the SVM model, a 10-fold cross validation was used to determine the expected accuracy of the model. We used this trained model to predict a class for all other cells in each liver section.

### INDUSTRIAL APPLICABILITY

The present invention allows assessment of unconventional genotoxic effects of chemicals in somatic cells of mice. In the animal model of the present invention any chemical can be tested for tumor-induction effect of an indirect mutagenic effect via the activation of L1 retrotransposons. Therefore, the present invention could be used, among others, in the field of toxicology, supporting chemical risk assessment toward toxicological endpoints not yet covered by known/standardized methods.

### REFERENCES

An, W., J. S. Han, S. J. Wheelan, E. S. Davis, C. E. Coombes, P. Ye, C. Triplett and J. D. Boeke (2006). “Active retrotransposition by a synthetic L1 element in mice.” Proc Natl Acad Sci USA 103(49): 18662-18667.
Aravin, A. A., R. Sachidanandam, D. Bourc'his, C. Schaefer, D. Pezic, K. F. Toth, T. Bestor and G. J. Hannon (2008). “A piRNA pathway primed by individual transposons is linked to de novo DNA methylation in mice.” Mol Cell 31(6): 785-799.
Ardeljan, D., J. P. Steranka, C. Liu, Z. L1, M. S. Taylor, L. M. Payer, M. Gorbounov, J. S. Sarnecki, V. Deshpande, R. H. Hruban, J. D. Boeke, D. Fenyo, P. H. Wu, A. Smogorzewska, A. J. Holland and K. H. Burns (2020). “Cell fitness screens reveal a conflict between LINE-1 retrotransposition and DNA replication.” Nat Struct Mol Biol 27(2): 168-178.
Beck, C. R., J. L. Garcia-Perez, R. M. Badge and J. V. Moran (2011). “LINE-1 elements in structural variation and disease.” Annu Rev Genomics Hum Genet 12: 187-215.
Belpomme, D., P. Irigaray, L. Hardell, R. Clapp, L. Montagnier, S. Epstein and A. J. Sasco (2007). “The multitude and diversity of environmental carcinogens.” Environ Res 105(3): 414-429.
Doucet-O'Hare, T. T., N. Rodic, R. Sharma, I. Darbari, G. Abril, J. A. Choi, J. Young Ahn, Y. Cheng, R. A. Anders, K. H. Burns, S. J. Meltzer and H. H. Kazazian, Jr. (2015). “LINE-1 expression and retrotransposition in Barrett's esophagus and esophageal carcinoma.” Proc Natl Acad Sci USA 112(35): E4894-4900.
Ewing, A. D., A. Gacita, L. D. Wood, F. Ma, D. Xing, M. S. Kim, S. S. Manda, G. Abril, G. Pereira, A. Makohon-Moore, L. H. Looijenga, A. J. Gillis, R. H. Hruban, R. A. Anders, K. E. Romans, A. Pandey, C. A. Iacobuzio-Donahue, B. Vogelstein, K. W. Kinzler, H. H. Kazazian, Jr. and S. Solyom (2015). “Widespread somatic L1 retrotransposition occurs early during gastrointestinal cancer evolution.” Genome Res 25(10): 1536-1545.
Feschotte, C. (2008). “Transposable elements and the evolution of regulatory networks.” Nat Rev Genet 9(5): 397-405.
Hamdorf, M., A. Idica, D. G. Zisoulis, L. Gamelin, C. Martin, K. J. Sanders and I. M. Pedersen (2015). “miR-128 represses L1 retrotransposition by binding directly to L1 RNA.” Nat Struct Mol Biol 22(10): 824-831.
Han, J. S. and J. D. Boeke (2004). “A highly active synthetic mammalian retrotransposon.” Nature 429(6989): 314-318.
Heras, S. R., S. Macias, M. Plass, N. Fernandez, D. Cano, E. Eyras, J. L. Garcia-Perez and J. F. Caceres (2013). “The Microprocessor controls the activity of mammalian retrotransposons.” Nat Struct Mol Biol 20(10): 1173-1181.
Herweijer, H., G. Zhang, V. M. Subbotin, V. Budker, P. Williams and J. A. Wolff (2001). “Time course of gene expression after plasmid DNA gene transfer to the liver.” J Gene Med 3(3): 280-291.
Jabbour, E., J. P. Issa, G. Garcia-Manero and H. Kantarjian (2008). “Evolution of decitabine development: accomplishments, ongoing investigations, and future strategies.” Cancer 112(11): 2341-2351.
Lantry, L. E., Z. Zhang, K. A. Crist, Y. Wang, G. J. Kelloff, R. A. Lubet and M. You (1999). “5-Aza-2′-deoxycytidine is chemopreventive in a 4-(methyl-nitrosamino)-1-(3-pyridyl)-1-butanone-induced primary mouse lung tumor model.” Carcinogenesis 20(2): 343-346.
Lehmann, K., C. Tschuor, A. Rickenbacher, J. H. Jang, C. E. Oberkofler, O. Tschopp, S. M. Schultze, D. A. Raptis, A. Weber, R. Graf, B. Humar and P. A. Clavien (2012). “Liver failure after extended hepatectomy in mice is mediated by a p21-dependent barrier to liver regeneration.” Gastroenterology 143(6): 1609-1619 e1604.
Li, X., J. Zhang, R. Jia, V. Cheng, X. Xu, W. Qiao, F. Guo, C. Liang and S. Cen (2013). “The MOV10 helicase inhibits LINE-1 mobility.” J Biol Chem 288(29): 21148-21160.
Lichtenstein, P., N. V. Holm, P. K. Verkasalo, A. Iliadou, J. Kaprio, M. Koskenvuo, E. Pukkala, A. Skytthe and K. Hemminki (2000). “Environmental and heritable factors in the causation of cancer—analyses of cohorts of twins from Sweden, Denmark, and Finland.” N Engl J Med 343(2): 78-85.
Livak, K. J. and T. D. Schmittgen (2001). “Analysis of relative gene expression data using real-time quantitative PCR and the 2(−Delta Delta C(T)) Method.” Methods 25(4): 402-408.
Madia, F., A. Worth, M. Whelan and R. Corvi (2019). “Carcinogenicity assessment: Addressing the challenges of cancer and chemicals in the environment.” Environ Int 128: 417-429.
Mahadevan, I. A., S. Kumar and M. R. S. Rao (2020). “Linker histone variant Hlt is closely associated with repressed repeat-element chromatin domains in pachytene spermatocytes.” Epigenetics Chromatin 13(1): 9.
McKerrow, W., X. Wang, C. Mendez-Dorantes, P. Mita, S. Cao, M. Grivainis, L. Ding, J. LaCava, K. H. Burns, J. D. Boeke and D. Fenyo (2022). “LINE-1 expression in cancer correlates with p53 mutation, copy number alteration, and S phase checkpoint.” Proc Natl Acad Sci USA 119(8).
Miki, Y., I. Nishisho, A. Horii, Y. Miyoshi, J. Utsunomiya, K. W. Kinzler, B. Vogelstein and Y. Nakamura (1992). “Disruption of the APC gene by a retrotransposal insertion of L1 sequence in a colon cancer.” Cancer Res 52(3): 643-645.
Mita, P., X. Sun, D. Fenyo, D. J. Kahler, D. L1, N. Agmon, A. Wudzinska, S. Keegan, J. S. Bader, C. Yun and J. D. Boeke (2020). “BRCA1 and S phase DNA repair pathways restrict LINE-1 retrotransposition in human cells.” Nat Struct Mol Biol 27(2): 179-191.
Naas, T. P., R. J. DeBerardinis, J. V. Moran, E. M. Ostertag, S. F. Kingsmore, M. F. Seldin, Y. Hayashizaki, S. L. Martin and H. H. Kazazian (1998). “An actively retrotransposing, novel subfamily of mouse L1 elements.” EMBO J 17(2): 590-597.
Naufer, M. N., A. V. Furano and M. C. Williams (2019). “Protein-nucleic acid interactions of LINE-1 ORF1p.” Semin Cell Dev Biol 86: 140-149.
Okudaira, N., M. Goto, R. Yanobu-Takanashi, M. Tamura, A. An, Y. Abe, S. Kano, S. Hagiwara, Y. Ishizaka and T. Okamura (2011). “Involvement of retrotransposition of long interspersed nucleotide element-1 in skin tumorigenesis induced by 7,12-dimethylbenz[a]anthracene and 12-O-tetradecanoylphorbol-13-acetate.” Cancer Sci 102(11): 2000-2006.
Okudaira, N., T. Okamura, M. Tamura, K. Iijma, M. Goto, A. Matsunaga, M. Ochiai, H. Nakagama, S. Kano, Y. Fujii-Kuriyama and Y. Ishizaka (2013). “Long interspersed element-1 is differentially regulated by food-borne carcinogens via the aryl hydrocarbon receptor.” Oncogene 32(41): 4903-4912.
Overturf, K., M. Al-Dhalimy, R. Tanguay, M. Brantly, C. N. Ou, M. Finegold and M. Grompe (1996). “Hepatocytes corrected by gene therapy are selected in vivo in a murine model of hereditary tyrosinaemia type I.” Nat Genet 12(3): 266-273.
Ozata, D. M., I. Gainetdinov, A. Zoch, D. O'Carroll and P. D. Zamore (2019). “PIWI-interacting RNAs: small RNAs with big functions.” Nat Rev Genet 20(2): 89-108.
Rannug, A. and U. Rannug (2018). “The tryptophan derivative 6-formylindolo[3,2-b]carbazole, FICZ, a dynamic mediator of endogenous aryl hydrocarbon receptor signaling, balances cell growth and differentiation.” Crit Rev Toxicol 48(7): 555-574.
Richardson, S. R., I. Narvaiza, R. A. Planegger, M. D. Weitzman and J. V. Moran (2014). “APOBEC3A deaminates transiently exposed single-strand DNA during LINE-1 retrotransposition.” Elife 3: e02008.
Rodic, N., R. Sharma, R. Sharma, J. Zampella, L. Dai, M. S. Taylor, R. H. Hruban, C. A. Iacobuzio-Donahue, A. Maitra, M. S. Torbenson, M. Goggins, M. Shih Je, A. S. Duffield, E. A. Montgomery, E. Gabrielson, G. J. Netto, T. L. Lotan, A. M. De Marzo, W. Westra, Z. A. Binder, B. A. Orr, G. L. Gallia, C. G. Eberhart, J. D.
Boeke, C. R. Harris and K. H. Burns (2014). “Long interspersed element-1 protein expression is a hallmark of many human cancers.” Am J Pathol 184(5): 1280-1286.
Rodic, N., J. P. Steranka, A. Makohon-Moore, A. Moyer, P. Shen, R. Sharma, Z. A. Kohutek, C. R. Huang, D. Ahn, P. Mita, M. S. Taylor, N. J. Barker, R. H. Hruban, C. A. Iacobuzio-Donahue, J. D. Boeke and K. H. Burns (2015). “Retrotransposon insertions in the clonal evolution of pancreatic ductal adenocarcinoma.” Nat Med 21(9): 1060-1064.
Rodriguez-Martin, B., E. G. Alvarez, A. Baez-Ortega, J. Zamora, F. Supek, J. Demeulemeester, M. Santamarina, Y. S. Ju, J. Temes, D. Garcia-Souto, H. Detering, Y. L1, J. Rodriguez-Castro, A. Dueso-Barroso, A. L. Bruzos, S. C. Dentro, M. G. Blanco, G. Contino, D. Ardeljan, M. Tojo, N. D. Roberts, S. Zumalave, P. A. W. Edwards, J. Weischenfeldt, M. Puiggros, Z. Chong, K. Chen, E. A. Lee, J. A. Wala, K. Raine, A. Butler, S. M. Waszak, F. C. P. Navarro, S. E. Schumacher, J. Monlong, F. Maura, N. Bolli, G. Bourque, M. Gerstein, P. J. Park, D. C. Wedge, R. Beroukhim, D. Torrents, J. O. Korbel, I. Martincorena, R. C. Fitzgerald, P. Van Loo, H. H. Kazazian, K. H. Burns, P. S. V. W. Group, P. J. Campbell, J. M. C. Tubio and P. Consortium (2020). “Pan-cancer analysis of whole genomes identifies driver rearrangements promoted by LINE-1 retrotransposition.” Nat Genet 52(3): 306-319.
Schutt, C., A. Hallmann, S. Hachim, I. Klockner, M. Valussi, A. Atzberger, J. Graumann, T. Braun and T. Boettger (2020). “Linc-MYH configures IN080 to regulate muscle stem cell numbers and skeletal muscle hypertrophy.” EMBO J 39(22): e105098.
Shukla, R., K. R. Upton, M. Munoz-Lopez, D. J. Gerhardt, M. E. Fisher, T. Nguyen, P. M. Brennan, J. K. Baillie, A. Collino, S. Ghisletti, S. Sinha, F. Iannelli, E. Radaelli, A. Dos Santos, D. Rapoud, C. Guettier, D. Samuel, G. Natoli, P. Carninci, F. D. Ciccarelli, J. L. Garcia-Perez, J. Faivre and G. J. Faulkner (2013). “Endogenous retrotransposition activates oncogenic pathways in hepatocellular carcinoma.” Cell 153(1): 101-111.
Skipper, K. A., P. R. Andersen, N. Sharma and J. G. Mikkelsen (2013). “DNA transposon-based gene vehicles—scenes from an evolutionary drive.” J Biomed Sci 20: 92.
Van Meter, M., M. Kashyap, S. Rezazadeh, A. J. Geneva, T. D. Morello, A. Seluanov and V. Gorbunova (2014). “SIRT6 represses LINE1 retrotransposons by ribosylating KAP1 but this repression fails with stress and age.” Nat Commun 5: 5011.
Vazquez, B. N., J. K. Thackray, N. G. Simonet, S. Chahar, N. Kane-Goldsmith, S. J. Newkirk, S. Lee, J. Xing, M. P. Verzi, W. An, A. Vaquero, J. A. Tischfield and L. Serrano (2019). “SIRT7 mediates L1 elements transcriptional repression and their association with the nuclear lamina.” Nucleic Acids Res 47(15): 7870-7885.
Wagstaff, B. J., M. Barnerssoi and A. M. Roy-Engel (2011). “Evolutionary conservation of the functional modularity of primate and murine LINE-1 elements.” PLoS One 6(5): e19672.
Yang, F., Y. Lan, R. R. Pandey, D. Homolka, S. L. Berger, R. S. Pillai, M. S. Bartolomei and P. J. Wang (2020). “TEX15 associates with MILI and silences transposable elements in male germ cells.” Genes Dev 34(11-12): 745-750.

1. A method for using a transgenic non-human vertebrate model animal, having somatic transgenic liver comprising an ORFeus reporter and providing sustained expression of the ORFeus reporter in the whole liver cell population of the liver in vivo, for testing a compound for its effect on modulation of L1 retrotransposition activity, wherein
the somatic transgenic liver cells of the liver of said animal comprise in their genome an expression cassette flanked by a pair of genomic integration sequences, said cassette comprising
a mammalian promoter, driving operably linked protein expression,
a first expression unit comprising
a positive selectable marker gene, which is a deficiency-complementing marker gene which provides a function in which the liver cells of the animal are deficient whereas expression of the deficiency-complementing selectable marker gene provides growth advantage to transgenic liver cells over the deficient liver cells and allows, once expressed in the liver cells, positive selection of the cells,
a second expression unit, comprising the ORFeus reporter element wherein said ORFeus reporter element comprises
a gene encoding LINE1-ORF1 (L1-ORF1 or ORF1 in short) and optionally a further gene encoding LINE1-ORF2 (L1-ORF2 or ORF2 in short), and
a retrotransposition reporter gene encoding a retrotransposition reporter protein,
wherein
said ORFeus reporter element is transcribed once the expression cassette is stably integrated into the genome of the transgenic liver cell and said ORF protein(s) is/are expressed, and
a retrotransposition reporter protein from said retrotransposition reporter gene is provided only when the ORFeus reporter element is subject to retrotransposition in the genome of the transgenic liver cell
said method comprising
administering said test compound to said transgenic animal of the invention,
measuring retrotransposition activity by the level of retrotransposition in the transgenic cells of the liver in the animal.

2. (canceled)

3. The method of a transgenic non-human vertebrate model animal according to claim 1, wherein the deficiency-complementing marker gene is the Fah selection marker gene and the transgenic non-human vertebrate model animal is a murine the liver of which is subjected to somatic genome editing.

4. (canceled)

5. The method of a transgenic non-human vertebrate model animal according to claim 1, wherein the somatic transgenic liver cells of said animal comprise in their genome an expression cassette flanked by a pair of genomic integration sequences, said cassette comprising
a mammalian promoter, driving operably linked protein expression by two sides of the promoter, a first side and a second side,
a first expression unit, under the control of the first side of the promoter, said first expression unit comprising
the positive selectable marker gene, which is a deficiency-complementing marker gene which provides a function in which the liver cells of the animal are deficient whereas expression of the deficiency-complementing selectable marker gene provides growth advantage to transgenic liver cells over the deficient liver cells and allows, once expressed in the liver cells, positive selection of the cells,
a second expression unit, under the control of the second side of the promoter, comprising the ORFeus reporter element wherein said ORFeus reporter element comprises
a gene encoding LINE1-ORF1 (L1-ORF1 or ORF1 in short) and optionally a further gene encoding LINE1-ORF2 (L1-ORF2 or ORF2 in short), and
a retrotransposition reporter gene encoding a retrotransposition reporter protein
wherein
said ORFeus reporter element is transcribed from the second side of the promoter once the expression cassette is stably integrated into the genome of the transgenic liver cell and said ORF protein(s) is/are expressed, and
a retrotransposition reporter protein from said retrotransposition reporter gene is expressed only when the ORFeus reporter element is subject to retrotransposition in the genome of the transgenic liver cell.

6. The use-method of the non-human vertebrate animal according to any of claim 1, wherein the effect of a test compound to modulate L1 retrotransposition activity in the vertebrate liver present in said animal in which the expression cassette is operable, is tested.

7. (canceled)

8. The method according to claim 1,
wherein said cassette comprises a mammalian bidirectional promoter, driving operably linked protein expression by two sides of the promoter, a first side and a second side, preferably a balanced expression.

9. The method according to claim 8, wherein the bidirectional promoter is a promoter which provides physiological expression level; preferably the expression level provided by the bidirectional promoter is more than 0.05 times, preferably 0.1 times and less than 10² times, preferably less than 50 times, preferably 10 times (particularly preferably 1-10 times) of that of a housekeeping gene, preferably coding Ribosomal Protein L27 protein sequence, wherein preferably the bidirectional promoter is a mammalian HADHA/B promoter, preferably a human HADHA/B promoter.

10. A transgenic non-human vertebrate model animal, having somatic transgenic liver comprising the ORFeus reporter and providing sustained expression of the ORFeus reporter in the whole liver cell population of the liver in vivo,
wherein the somatic transgenic liver cells of said animal comprise in their genome an expression cassette flanked by a pair of genomic integration sequences, said cassette comprising
a mammalian promoter, driving operably linked protein expression,
a first expression unit, said first expression unit comprising
a positive selectable marker gene, which is a deficiency-complementing marker gene which provides a function in which the liver cells of the animal are deficient whereas expression of the deficiency-complementing selectable marker gene provides growth advantage to transgenic liver cells over the deficient liver cells and allows, once expressed in the liver cells, positive selection of the cells,
a second expression unit, comprising the ORFeus reporter element wherein said ORFeus reporter element comprises
a gene encoding LINE1-ORF1 (L1-ORF1 or ORF1 in short) and optionally a further gene encoding LINE1-ORF2 (L1-ORF2 or ORF2 in short), and
a retrotransposition reporter gene encoding a retrotransposition reporter protein
wherein
said ORFeus reporter element is transcribed from the second side of the promoter once the expression cassette is stably integrated into the genome of the transgenic liver cell and said ORF protein(s) is/are expressed,
and
a retrotransposition reporter protein from said retrotransposition reporter gene is provided (i.e. expressed) only when the ORFeus reporter element is subject to retrotransposition in the genome of the transgenic liver cell.

11. A transgenic non-human vertebrate model animal, having somatic transgenic liver comprising the ORFeus reporter and providing sustained expression of the ORFeus reporter in the whole liver cell population of the liver in vivo,
wherein the somatic transgenic liver cells of said animal comprise in their genome an expression cassette flanked by a pair of genomic integration sequences, said cassette comprising
a mammalian promoter, driving operably linked protein expression,
a first expression unit, said first expression unit comprising
a positive selectable marker gene, which is a deficiency-complementing marker gene which provides a function in which the liver cells of the animal are deficient whereas expression of the deficiency-complementing selectable marker gene provides growth advantage to transgenic liver cells over the deficient liver cells and allows, once expressed in the liver cells, positive selection of the cells,
a second expression unit, comprising the ORFeus reporter element wherein said ORFeus reporter element comprises
a gene encoding LINE1-ORF1 (L1-ORF1 or ORF1 in short) and optionally a further gene encoding LINE1-ORF2 (L1-ORF2 or ORF2 in short), and
a retrotransposition reporter gene encoding a retrotransposition reporter protein
wherein
said ORFeus reporter element is transcribed from the second side of the promoter once the expression cassette is stably integrated into the genome of the transgenic liver cell and said ORF protein(s) is/are expressed, and
a retrotransposition reporter protein from said retrotransposition reporter gene is provided (i.e. expressed) only when the ORFeus reporter element is subject to retrotransposition in the genome of the transgenic liver cell.

12. The transgenic non-human vertebrate model animal according to claim 11, wherein the deficiency-complementing marker gene is the Fah selection marker gene and the transgenic non-human vertebrate model animal is a murine the liver of which is subjected to somatic genome editing.

13. The transgenic non-human vertebrate model animal according to claim 11, wherein said cassette comprises a mammalian, preferably human bidirectional promoter, driving operably linked protein expression by two sides of the promoter, a first side and a second side.

14. The transgenic non-human vertebrate model animal according to claim 13, wherein
the bidirectional promoter is a promoter which provides physiological expression level; preferably the expression level provided by the bidirectional promoter is more than 0.05 times, preferably 0.1 times and less than 10² times, preferably less than 50 times, preferably 10 times (particularly preferably 0.1-10 times) of that of a housekeeping gene, preferably coding Ribosomal Protein L27 protein sequence.

15. The transgenic non-human vertebrate model animal according to claim 14, wherein the bidirectional promoter is a mammalian HADHA/B promoter, preferably a human HADHA/B promoter.

16. A method for preparing a transgenic vertebrate, preferably a mammalian animal for use in measuring the level of modulation of L1 retrotransposition activity in the liver of said animal, wherein
the liver of said animal is populated with transgenic liver cells, comprising an expression construct stably integrated in their genome, said construct comprising a positive selectable marker gene which is a deficiency-complementing marker gene, and which provides a function in which the liver cells of the animal are deficient, whereas expression of the deficiency-complementing selectable marker gene provides growth advantage to transgenic liver cells over the deficient liver cells,
said method comprising the steps of
providing a vertebrate, preferably a mammalian animal in which the selectable marker gene is deficient (dysfunctional), wherein in lack of such selectable marker gene function the liver cells of the animal are impaired,
providing a population of transgenic liver cells in the animal by co-administering an expression vector comprising an expression construct comprising a
a first expression unit, said first expression unit comprising a deficiency-complementing selectable marker gene allowing, once expressed in the liver cells, positive selection of the cells, and being useful for in vivo somatic transgenesis of the liver,
a second expression unit, comprising an ORFeus reporter element wherein said ORFeus reporter element comprises
a gene encoding LINE1-ORF1 (L1-ORF1 or ORF1 in short), and optionally LINE1-ORF2,
a retrotransposition reporter gene encoding a retrotransposition reporter protein,
wherein said ORFeus reporter element is transcribed once the expression cassette is stably integrated into the genome of the liver cell
a helper vector
thereby obtaining said population comprising the expression unit of the expression vector functionally integrated into their chromosomes, wherein both the selectable marker gene and the gene encoding LINE1-ORF1 and optionally LINE1-ORF2 is expressed,
providing selective advantage to the transgenic cells having the deficiency-complementing selectable marker gene integrated into their genome,
allowing the transgenic liver cells to proliferate in the liver, whereas the amount of impaired liver cells is decreasing until transgenic liver is obtained in the mammalian animal.

17. The method according to claim 16, wherein the deficiency-complementing marker gene is the Fah selection marker gene and the transgenic non-human vertebrate model animal is a murine the liver of which is subjected to somatic genome editing, and wherein preferably the transgenic liver cells are prepared by administering the expression vector of the invention and a helper vector comprising an expression construct which, when expressed in the same cell in which the expression vector is present, promotes integration of the expression unit into the genome of the cell and wherein preferably the vectors are co-administered by a hydrodynamic injection into the animals.

18. The method according to claim 16, wherein said expression construct comprises a mammalian, preferably human bidirectional promoter, driving operably linked protein expression by two sides of the promoter, a first side and a second side.

19. (canceled)

20. (canceled)

21. An expression vector operable in vertebrate liver cells, preferably mammalian liver cells, preferably hepatocytes, said vector comprising an expression cassette flanked by a pair of genomic integration sequences, said cassette comprising
a mammalian, preferably human bidirectional promoter, driving operably linked protein expression by two sides of the promoter, a first side and a second side,
a first expression unit, under the control of the first side of the promoter, said first expression unit comprising a positive selectable marker gene, which is a deficiency-complementing marker gene and is useful for in vivo somatic transgenesis of the liver of a vertebrate animal and, once expressed in the liver cells, for positive selection of the cells,
a second expression unit, under the control of the second side of the promoter, comprising an ORFeus reporter element wherein said ORFeus reporter element comprises
a gene encoding LINE1-ORF1 (L1-ORF1 or ORF1 in short) and optionally a further gene encoding LINE1-ORF2 (L1-ORF2 or ORF2 in short), and
a retrotransposition reporter gene encoding a retrotransposition reporter protein,
wherein said ORFeus reporter element is transcribed from the second side of the promoter once the expression cassette is stably integrated into the genome of the liver cell, being a transgenic liver cell and said ORF protein(s) is/are expressed, a retrotransposition reporter protein from said retrotransposition reporter gene is provided only when the ORFeus reporter element is subject to retrotransposition in the genome of the transgenic liver cell.

22. The expression vector of claim 21, wherein the deficiency-complementing marker gene is the Fah gene.

23. The expression vector of claim 21 wherein the ORFeus reporter element comprises, in reverse orientation, an expression unit for the retrotransposition reporter gene,
said expression unit for the retrotransposition reporter gene comprising a first exon, a second exon and between them an intron which is removed in the retrotransposition process wherein a retrotransposition reporter protein is provided only when a retrotransposition occurs.

24. The expression vector of claim 23 wherein the expression unit for the retrotransposition reporter gene in reverse orientation comprises
a first exon of a visible marker gene, preferably a fluorescent marker gene and,
a second exon of the visible marker gene, preferably the fluorescent marker gene
wherein upon retrotransposition, once linked with the polypeptide encoded by the second exon, a visible retrotransposition reporter, preferably a fluorescent protein, is expressed from the visible marker gene.

25. The expression vector of claim 23 wherein the second exon has, operably linked thereto, a coding region for a peptide tag which serves as an epitope for an antibody specific for the particular peptide tag, and/or wherein the intron is relocated to increase the length of the second exon and decrease the length of the first exon thereby providing an epitope within the second exon which serves as an epitope for an antibody specific for the second exon.

26. (canceled)

27. The expression vector of claim 21 wherein the ORFeus reporter element comprises in sense (forward) orientation an ORF protein expression unit comprising the gene encoding one or two ORF protein(s) and the 3′UTR and/or wherein the ORFeus reporter element comprises, from the second side of the promoter, a LINE1 ORF1 coding sequence (L1-ORF1) and optionally a LINE1 ORF2 coding sequence (L1-ORF2), a 3′ untranslated region (3′UTR), and, in reverse orientation, an expression unit for the retrotransposition reporter gene.

28. (canceled)

29. (canceled)

30. (canceled)

31. The expression vector of claim 21 wherein the bidirectional promoter is a mammalian HADHA/B promoter, preferably a human HADHA/B promoter.

32. The expression vector of claim 31 wherein the expression level provided by the HADHA/B promoter is in the physiological range of expression, i.e. in comparison with the expression level of the Rpl27 housekeeping gene the expression level provided by the HADHA/B promoter (i.e. the expression level of the genes driven by the HADHA/B promoter) is at most 2 orders of magnitude higher than the expression of the Rpl27 housekeeping gene.

33. (canceled)

34. (canceled)

35. The expression vector of claim 21 wherein the vector also comprises an intron comprising integration site to insert one or more silencer sequence(s), optionally said silencer sequence being inserted into said integration site, wherein the intron (EF1-intron) is at least 400, preferably 500, more preferably 600 nucleotide long and has 5′ and 3′ splice sites and a branch site of the intron and has at least 70%, preferably at least 80%, identity with the corresponding sequence part of the human eukaryotic translation elongation factor 1 alpha 1 (EEF1A1).

36. (canceled)

37. The expression vector of claim 35 wherein gene silencing is artificial microRNA-based (amiR-based) gene silencing.

38. (canceled)
